(19) United States
(12) Patent Application Publication
(10) Pub. No.: US 2008/0051435 A1
(43) Pub. Date: Feb. 28, 2008

(54) Crystalline and amorphous forms of tiagabine

(75) Inventors: Scott L. Childs, Atlanta, GA (US); Karen S. Gushurst, West Lafayette, IN (US); R. Curtis Haltiwanger, West Chester, PA (US); Robert E. McKean, Chester Springs, PA (US); Donglai Yang, West Lafayette, IN (US)

Correspondence Address: CEPHALON, INC., 41 MOORES ROAD, PO BOX 4011, FRAZER, PA, 19355, US

(73) Assignee: Cephalon, Inc., Frazer, PA (US)

(21) Appl. No.: 11/893,524

(22) Filed: Aug. 16, 2007

Related U.S. Application Data
(60) Provisional application No. 60/838,661, filed on Aug. 18, 2006.

(51) Int. Cl. C07D 409/14; A61K 31/4535

(52) U.S. Cl. 514/326; 546/212

(57) ABSTRACT

The present invention provides 24 new forms of tiagabine, including 22 new crystalline forms of tiagabine and its salts, an amorphous form of tiagabine free base, and a cocrystal form of tiagabine hydrochloride with 2-furancarboxylic acid. The present invention further provides a process for preparing each of the new tiagabine forms. The present invention further provides a pharmaceutical composition containing at least one of the new tiagabine forms, and a process for the preparation thereof. The present invention further provides a method of treating a disease related to GABA uptake in a mammal, comprising the step of administering to the mammal a therapeutically effective amount of at least one of the new tiagabine forms.

### BACKGROUND OF THE INVENTION

1. Technical Field

This invention relates to crystalline and amorphous forms of tiagabine free base and tiagabine salts.

2. Background Art

Tiagabine ((−)-(R)-1-(4,4-bis(3-methyl-2-thienyl)-3-butenyl)-3-piperidinecarboxylic acid; CAS # 115103-54-3) is a gamma-aminobutyric acid (GABA) uptake inhibitor. Tiagabine is often used as an adjunctive therapy in adults and children twelve (12) years and older for treatment of partial seizures, and is marketed in the form of its hydrochloride salt under the trade name GABITRIL® (Cephalon, Inc., Frazer, Pa.). Tiagabine hydrochloride has the following chemical structure:

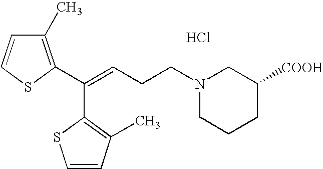

U.S. Pat. No. 5,010,090 (the '090 patent) discloses crystalline tiagabine hydrochloride prepared by crystallization from ethyl acetate, isopropanol, acetone, or water. The '090 patent does not disclose the x-ray diffraction pattern, solvent content, differential scanning calorimetry (DSC) pattern, thermogravimetric analysis (TGA), or nuclear magnetic resonance (NMR) spectrum of the prepared tiagabine hydrochloride.

U.S. Pat. No. 5,354,760 (the '760 patent) discloses a monohydrate crystalline form of tiagabine hydrochloride. This crystalline form is referred to herein as tiagabine hydrochloride monohydrate or tiagabine hydrochloride Form A. The '760 patent discloses the preparation of tiagabine hydrochloride Form A by crystallizing tiagabine hydrochloride from water or aqueous hydrochloric acid. The '760 patent provides X-ray powder diffraction (XRPD), ¹H-NMR, infrared (IR) spectroscopy, DSC, and water content characterization data for the obtained crystalline form. The '760 patent states that crystallizing tiagabine hydrochloride from solvents such as ethyl acetate, acetonitrile, butyl acetate, toluene, acetone, or dichloromethane gives products containing varying amounts of the used crystallizing solvent. However, no organic solvent solvate crystalline form of tiagabine hydrochloride is disclosed.

U.S. Pat. No. 5,958,951 (the '951 patent) discloses an anhydrous crystalline form of tiagabine hydrochloride. This crystalline form is referred to herein as tiagabine hydrochloride anhydrous or tiagabine hydrochloride Form B. The '951 patent discloses the preparation of tiagabine hydrochloride Form B by crystallizing tiagabine hydrochloride from aqueous hydrochloric acid under specified conditions. The '951 patent provides XRPD, DSC, TGA, and water content characterization data for tiagabine hydrochloride Form B. The '951 patent states that crystallizing tiagabine hydrochloride from ethyl acetate gives products containing unwanted amounts of the crystallizing solvent; and the use of other organic solvents often results in the formation of solvates of tiagabine hydrochloride. However, no organic solvent solvate crystalline form of tiagabine hydrochloride is disclosed.

WO 2005/092886 A1 (the '886 application) discloses an amorphous form of tiagabine hydrochloride prepared by spray drying a methanol solution of tiagabine hydrochloride. XRPD, IR, and DSC data are provided. No crystalline form is disclosed.

There is a continuing need for additional crystalline and amorphous forms of tiagabine free base and tiagabine salts.

### SUMMARY OF THE INVENTION

The present invention provides a crystalline form of tiagabine chosen from tiagabine free base Form A, tiagabine free base Form B, tiagabine free base Form C, tiagabine free base Form D, tiagabine free base Form E, tiagabine free base Form F, tiagabine free base Form G, tiagabine free base Form H, tiagabine camphorate Form A, tiagabine hydrobromide Form A, tiagabine dl-malate Form A, tiagabine d-malate Form A, tiagabine tartrate Form A, tiagabine hydrochloride Form G, tiagabine hydrochloride Form K, tiagabine hydrochloride Form L, tiagabine hydrochloride Form N, tiagabine hydrochloride Form O, tiagabine hydrochloride Form R, tiagabine hydrochloride Form U, tiagabine hydrochloride Form V, tiagabine hydrochloride Form AC, and Crystalline Form A of tiagabine hydrochloride cocrystal with 2-furancarboxylic acid. Preferably, the crystalline form of tiagabine has a purity of at least about 50% (w/w).

Preferably, the crystalline form of tiagabine exhibits an x-ray powder diffraction pattern having characteristic peaks as set forth in the following Table A:

TABLE A
Characteristic XRPD Peaks of Tiagabine Crystalline Forms

| Tiagabine | Form | Characteristic XRPD Peaks (±0.2 degrees 2θ) | | | | | | | | | |
|---|---|---|---|---|---|---|---|---|---|---|---|
| free base | A | 6.5 | 8.1 | 12.6 | 17.4 | 19.0 | 19.5 | 22.9 | 25.8 | 27.2 | — |
| free base | B | 15.0 | 15.4 | 17.3 | 21.3 | 22.5 | 24.8 | — | — | — | — |
| free base | C | 4.9 | 6.1 | 7.8 | 9.9 | 12.2 | 12.9 | — | — | — | — |
| free base | D | 5.7 | 6.1 | 10.0 | 12.2 | 15.8 | 16.9 | — | — | — | — |
| free base | E | 9.5 | 13.1 | 14.3 | 16.1 | 18.7 | 22.5 | — | — | — | — |
| free base | F | 6.3 | 8.0 | 10.0 | 10.5 | 16.2 | 21.1 | 21.8 | — | — | — |
| free base | G | 6.0 | 7.6 | 9.7 | 15.4 | 16.1 | 18.1 | 18.5 | 19.0 | 24.7 | — |
| free base | H | 15.8 | 16.8 | 20.7 | — | — | — | — | — | — | — |
| camphorate | A | 5.9 | 9.8 | 12.0 | 14.0 | 15.4 | 18.4 | 21.2 | — | — | — |
| hydrobromide | A | 3.9 | 7.8 | 12.8 | 14.2 | 14.4 | 15.7 | 21.5 | 21.8 | — | — |
| dl-malate | A | 4.2 | 11.3 | 11.9 | 15.5 | 15.9 | 18.7 | 19.2 | — | — | — |
| d-malate | A | 4.2 | 11.3 | 11.9 | 15.9 | 17.0 | 18.7 | 21.1 | 23.8 | — | — |
| Tartrate | A | 4.1 | 11.5 | 12.6 | 13.6 | 16.5 | 16.7 | 21.5 | 24.6 | — | — |
| hydrochloride | G | 3.9 | 14.7 | 16.0 | 16.9 | 20.5 | 25.5 | 28.1 | — | — | — |
| hydrochloride | K | 5.7 | 13.3 | 16.6 | 20.1 | 20.6 | 23.6 | 24.5 | 24.9 | — | — |
| hydrochloride | L | 7.7 | 12.5 | 14.5 | 17.1 | 21.1 | 21.8 | 24.6 | 25.1 | 26.2 | 28.0 |
| hydrochloride | N | 14.1 | 14.5 | 15.6 | 17.1 | 19.6 | 22.6 | 23.2 | 23.8 | 24.7 | 25.0 |
| hydrochloride | O | 12.6 | 14.6 | 16.4 | 18.6 | 18.9 | 23.3 | 24.3 | 25.9 | — | — |
| hydrochloride | R | 10.8 | 13.0 | 15.3 | 16.7 | 17.8 | 22.2 | 25.4 | 26.9 | 28.0 | 32.2 |
| hydrochloride | U | 12.6 | 14.4 | 16.4 | 16.9 | 21.2 | 21.6 | 22.9 | 23.9 | 26.6 | 27.6 |
| hydrochloride | V | 7.4 | 11.6 | 12.9 | 15.8 | 16.1 | 18.5 | 19.4 | 21.2 | 23.9 | 26.4 |
| hydrochloride | AC | 7.8 | 8.5 | 12.4 | 14.7 | 15.3 | 15.8 | 17.0 | 18.2 | 22.9 | 25.0 |
| hydrochloride co- | A | 7.5 | 11.6 | 14.7 | 17.2 | 21.7 | 22.9 | 26.6 | — | — | — |
| crystal with 2-furan- | | | | | | | | | | | |
| carboxylic acid | | | | | | | | | | | |

Preferably, the crystalline form of tiagabine has a purity of at least about 50% (w/w).

Preferably, the crystalline form of tiagabine is chosen from tiagabine free base Forms A, B, C, D, E, F, G, and H, exhibiting an x-ray powder diffraction pattern having characteristic peaks as set forth in the following Table 1:

TABLE 1
Characteristic XRPD Peaks of Tiagabine Free Base Crystalline Forms

| Form | Characteristic XRPD Peaks (±0.2 degrees 2θ) | | | | | | | | |
|---|---|---|---|---|---|---|---|---|---|
| A | 6.5 | 8.1 | 12.6 | 17.4 | 19.0 | 19.5 | 22.9 | 25.8 | 27.2 |
| B | 15.0 | 15.4 | 17.3 | 21.3 | 22.5 | 24.8 | — | — | — |
| C | 4.9 | 6.1 | 7.8 | 9.9 | 12.2 | 12.9 | — | — | — |
| D | 5.7 | 6.1 | 10.0 | 12.2 | 15.8 | 16.9 | — | — | — |
| E | 9.5 | 13.1 | 14.3 | 16.1 | 18.7 | 22.5 | — | — | — |
| F | 6.3 | 8.0 | 10.0 | 10.5 | 16.2 | 21.1 | 21.8 | — | — |
| G | 6.0 | 7.6 | 9.7 | 15.4 | 16.1 | 18.1 | 18.5 | 19.0 | 24.7 |
| H | 15.8 | 16.8 | 20.7 | — | — | — | — | — | — |

Preferably, the crystalline form of tiagabine is a tiagabine salt chosen from tiagabine camphorate Form A, tiagabine hydrobromide Form A, tiagabine dl-malate Form A, tiagabine d-malate Form A, and tiagabine tartrate Form A, exhibiting an x-ray powder diffraction pattern having characteristic peaks as set forth in the following Table 2:

TABLE 2
Characteristic XRPD Peaks of Tiagabine Salt Crystalline Forms

| Tiagabine Salt | Form | Characteristic XRPD Peaks (±0.2 degrees 2θ) | | | | | | | |
|---|---|---|---|---|---|---|---|---|---|
| Tiagabine camphorate | A | 5.9 | 9.8 | 12.0 | 14.0 | 15.4 | 18.4 | 21.2 | — |
| Tiagabine hydrobromide | A | 3.9 | 7.8 | 12.8 | 14.2 | 14.4 | 15.7 | 21.5 | 21.8 |
| Tiagabine dl-malate | A | 4.2 | 11.3 | 11.9 | 15.5 | 15.9 | 18.7 | 19.2 | — |
| Tiagabine d-malate | A | 4.2 | 11.3 | 11.9 | 15.9 | 17.0 | 18.7 | 21.1 | 23.8 |
| Tiagabine tartrate | A | 4.1 | 11.5 | 12.6 | 13.6 | 16.5 | 16.7 | 21.5 | 24.6 |

Preferably, the crystalline form of tiagabine is a tiagabine hydrochloride salt chosen from Forms G, K, L, N, O, R, U, V, and AC, exhibiting an x-ray powder diffraction pattern having characteristic peaks as set forth in the following Table 3:

TABLE 3
Characteristic XRPD Peaks of Tiagabine HCl Crystalline Forms

| Form | Characteristic XRPD Peaks (±0.2 degrees 2θ) | | | | | | | | | |
|---|---|---|---|---|---|---|---|---|---|---|
| G | 3.9 | 14.7 | 16.0 | 16.9 | 20.5 | 25.5 | 28.1 | — | — | — |
| K | 5.7 | 13.3 | 16.6 | 20.1 | 20.6 | 23.6 | 24.5 | 24.9 | — | — |
| L | 7.7 | 12.5 | 14.5 | 17.1 | 21.1 | 21.8 | 24.6 | 25.1 | 26.2 | 28.0 |
| N | 14.1 | 14.5 | 15.6 | 17.1 | 19.6 | 22.6 | 23.2 | 23.8 | 24.7 | 25.0 |
| O | 12.6 | 14.6 | 16.4 | 18.6 | 18.9 | 23.3 | 24.3 | 25.9 | — | — |
| R | 10.8 | 13.0 | 15.3 | 16.7 | 17.8 | 22.2 | 25.4 | 26.9 | 28.0 | 32.2 |
| U | 12.6 | 14.4 | 16.4 | 16.9 | 21.2 | 21.6 | 22.9 | 23.9 | 26.6 | 27.6 |
| V | 7.4 | 11.6 | 12.9 | 15.8 | 16.1 | 18.5 | 19.4 | 21.2 | 23.9 | 26.4 |
| AC | 7.8 | 8.5 | 12.4 | 14.7 | 15.3 | 15.8 | 17.0 | 18.2 | 22.9 | 25.0 |

More preferably, the crystalline form of tiagabine is a tiagabine hydrochloride salt chosen from Forms G, L, O and V.

Preferably, the crystalline form of tiagabine is Crystalline Form A of tiagabine hydrochloride cocrystal with 2-furancarboxylic acid, exhibiting an x-ray powder diffraction pattern having characteristic peaks at 7.5, 11.6, 14.7, 17.2, 21.7, 22.9 and 26.6±0.2 degrees 2θ.

The present invention further provides tiagabine free base amorphous. Preferably, the tiagabine free base amorphous has a purity of at least about 50% (w/w).

The present invention further provides a pharmaceutical composition comprising one or more of the above crystalline forms of tiagabine and one or more pharmaceutically acceptable excipients.

The present invention further provides a pharmaceutical composition comprising tiagabine free base amorphous and one or more pharmaceutically acceptable excipients.

The present invention further provides a process for preparing a crystalline form of tiagabine comprising the steps of:
(a) crystallizing tiagabine free base from ethanol to provide tiagabine free base Form A; or
(b) slurrying tiagabine free base in a mixture of hexane, diisopropylether, and ethanol to provide tiagabine free base Form A; or
(c) drying tiagabine free base Form A under vacuum to provide tiagabine free base Form B; or
(d) crystallizing tiagabine free base from a solvent selected from isopropanol, acetonitrile, and ethanol to provide tiagabine free base Form C; or
(e) crystallizing tiagabine free base from a mixture of isopropanol and cyclohexane to provide tiagabine free base Form C; or
(f) crystallizing tiagabine free base from a mixture of methyl ethyl ketone and 2,2,2-trifluoroethanol to provide tiagabine free base Form C; or
(g) crystallizing tiagabine free base from a mixture of 2,2,2-trifluoroethanol and at least one solvent chosen from methyl ethyl ketone and isopropyl ether to provide tiagabine free base Form D; or
(h) crystallizing tiagabine free base from a mixture of propionitrile and t-butyl alcohol to provide tiagabine free base Form E; or
(i) crystallizing tiagabine free base from a mixture of methyl ethyl ketone and 2,2,2-trifluoroethanol to provide tiagabine free base Form E; or
(j) crystallizing tiagabine free base from acetonitrile to provide tiagabine free base Form E; or
(k) crystallizing tiagabine free base from a mixture of 2,2,2-trifluoroethanol, methyl ethyl ketone, and propyl ether to provide tiagabine free base Form E; or
(l) crystallizing tiagabine free base from a mixture of methanol and 2-propyl ether to provide tiagabine free base Form F; or
(m) crystallizing tiagabine free base from 2-butanol to provide tiagabine free base Form G; or
(n) crystallizing tiagabine free base from I -propanol to provide tiagabine free base Form H; or
(o) preparing a solution of tiagabine free base and (+)-camphoric acid in methanol, and crystallizing tiagabine camphorate Form A from the solution; or
(p) preparing a solution of tiagabine free base and (+)-camphoric acid in methanol and acetonitrile or ethyl acetate, and crystallizing tiagabine camphorate Form A from the solution; or
(q) preparing a solution of tiagabine free base and hydrobromic acid in a mixture of ethyl acetate and acetonitrile, and crystallizing tiagabine hydrobromide Form A from the solution; or
(r) preparing a solution of tiagabine free base and hydrobromic acid in a mixture of ethyl acetate, acetonitrile and 2-propyl ether, and crystallizing tiagabine hydrobromide Form A from the solution; or
(s) preparing a solution of tiagabine free base and dl-malic acid in a mixture of ethyl acetate, acetonitrile and methanol, and crystallizing tiagabine dl-malate Form A from the solution; or
(t) preparing a solution of tiagabine free base and d-malic acid in a mixture of ethyl acetate and acetonitrile, and crystallizing tiagabine d-malate Form A from the solution; or
(u) preparing a solution of tiagabine free base and d-malic acid in a mixture of ethyl acetate, acetonitrile and methanol, and crystallizing tiagabine d-malate Form A from the solution; or
(v) preparing a solution of tiagabine free base and d-malic acid in a mixture of ethyl acetate and acetonitrile, crystallizing tiagabine d-malate Form A from the solution, and slurrying the crystallized tiagabine d-malate Form A in ether; or
(w) preparing a solution of tiagabine free base and L-(+)-tartaric acid in a mixture of methanol and acetonitrile, and crystallizing tiagabine tartrate Form A from the solution; or
(x) preparing a solution of tiagabine free base and L-(+)-tartaric acid in a mixture of methanol, acetonitrile and ethyl acetate, and crystallizing tiagabine tartrate Form A from the solution; or
(y) preparing a solution of tiagabine free base and L-(+)-tartaric acid in a mixture of acetone and ethyl acetate, and crystallizing tiagabine tartrate Form A from the solution; or
(z) preparing a solution of tiagabine free base and L-(+)-tartaric acid in a mixture of tetrahydrofuran and 2-propanol, and crystallizing tiagabine tartrate Form A from the solution; or
(aa) preparing a mixture of tiagabine hydrochloride and 2-furancarboxylic acid, and grinding the mixture to form crystalline Form A of tiagabine hydrochloride cocrystal with 2-furancarboxylic acid; or
(bb) preparing a mixture of tiagabine hydrochloride, 2-furancarboxylic acid and methanol, and grinding the mixture to form crystalline Form A of tiagabine hydrochloride cocrystal with 2-furancarboxylic acid; or
(cc) preparing a mixture of tiagabine hydrochloride monohydrate and 2-furancarboxylic, and grinding the mixture to form crystalline Form A of tiagabine hydrochloride cocrystal with 2-furancarboxylic acid; or
(dd) crystallizing tiagabine hydrochloride from chloroform to provide tiagabine hydrochloride Form G; or
(ee) crystallizing tiagabine hydrochloride from chloroform to provide tiagabine hydrochloride Form K; or
(ff) crystallizing tiagabine hydrochloride from nitromethane to provide tiagabine hydrochloride Form L; or
(gg) crystallizing tiagabine hydrochloride from benzonitrile to provide tiagabine hydrochloride Form N; or
(hh) heating tiagabine hydrochloride monohydrate to provide tiagabine hydrochloride Form O; or
(ii) slurrying tiagabine hydrochloride monohydrate in nitromethane to provide tiagabine hydrochloride Form R; or
(jj) slurrying tiagabine hydrochloride monohydrate in 1,2-dichloroethane to provide tiagabine hydrochloride Form U; or
(kk) slurrying tiagabine hydrochloride monohydrate in 1,2-dimethoxyethane to provide tiagabine hydrochloride Form V; or
(ll) crystallizing tiagabine hydrochloride from cyclohexanol to provide tiagabine hydrochloride Form AC.

The present invention further provides a process for preparing an amorphous form of tiagabine free base comprising the step of:
(a) evaporating a solution of tiagabine free base in a solvent selected from 1,4-dioxane and isopropanol to provide tiagabine free base amorphous; or
(b) adding propyl ether to a solution of tiagabine free base in 1,4-dioxane to provide tiagabine free base amorphous; or
(c) precipitating tiagabine free base from an acetonitrile solution to provide tiagabine free base amorphous.

### BRIEF DESCRIPTION OF THE DIAGRAMS

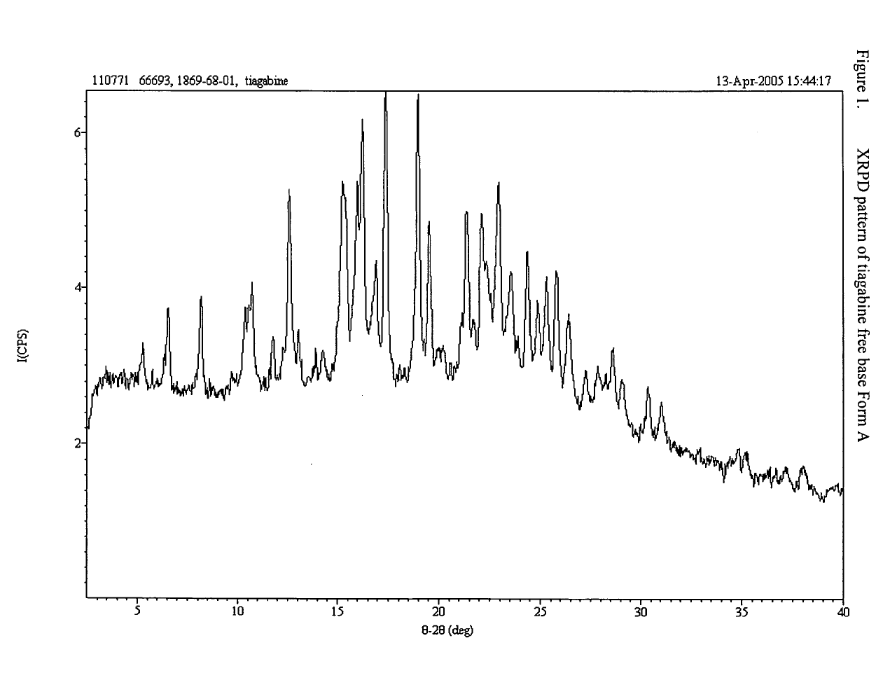

FIG. 1 depicts an x-ray powder diffraction (XRPD) pattern of tiagabine free base Form A.

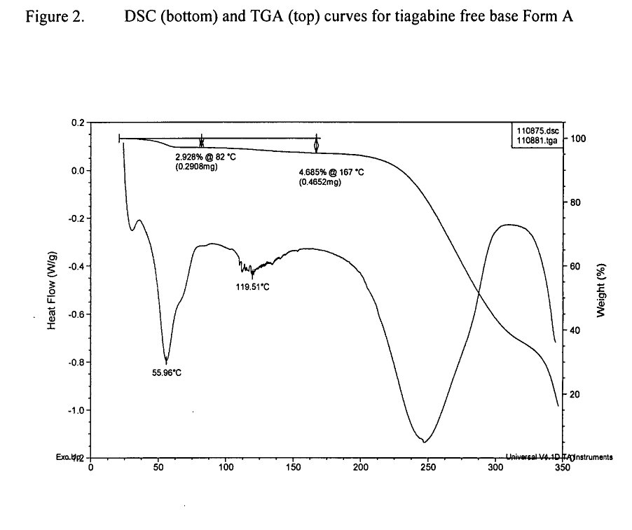

FIG. 2 depicts a differential scanning calorimetry (DSC) curve and a thermogravimetric analysis (TGA) curve for tiagabine free base Form A.

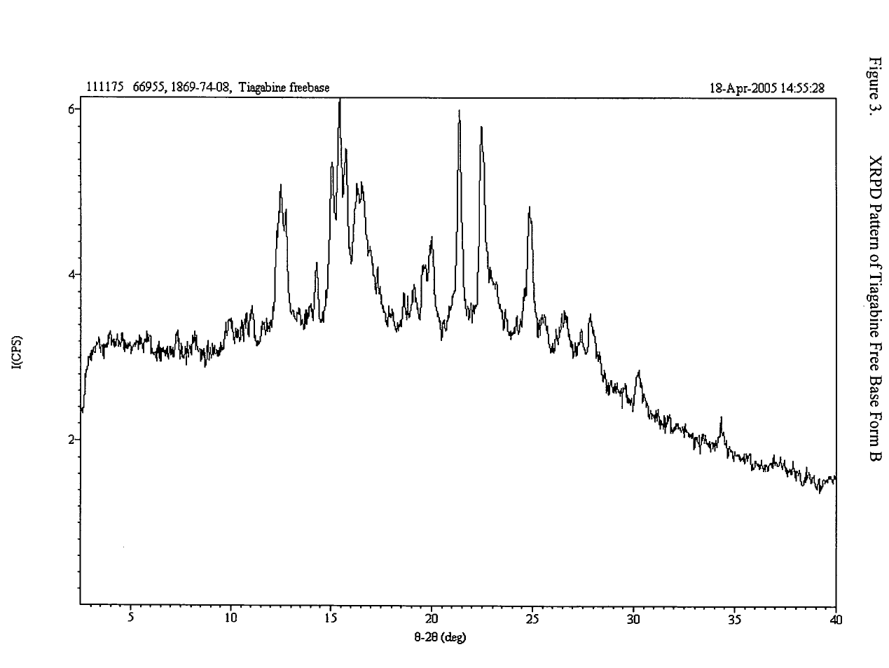

FIG. 3 depicts an XRPD pattern of tiagabine free base Form B.

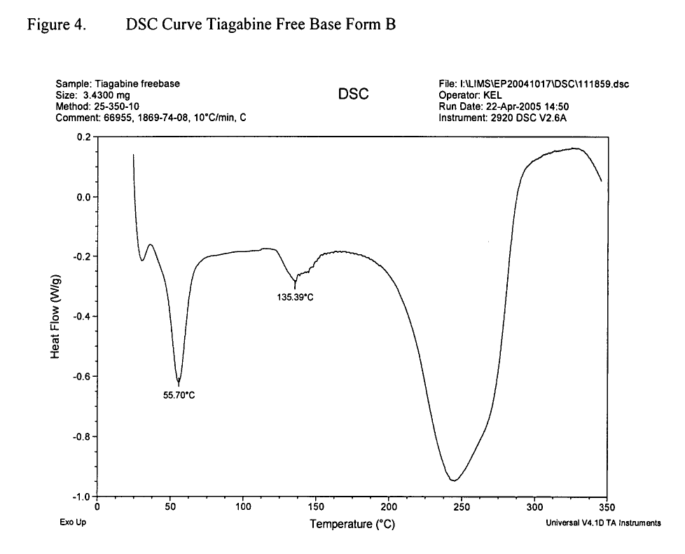

FIG. 4 depicts a DSC curve of tiagabine free base Form B.

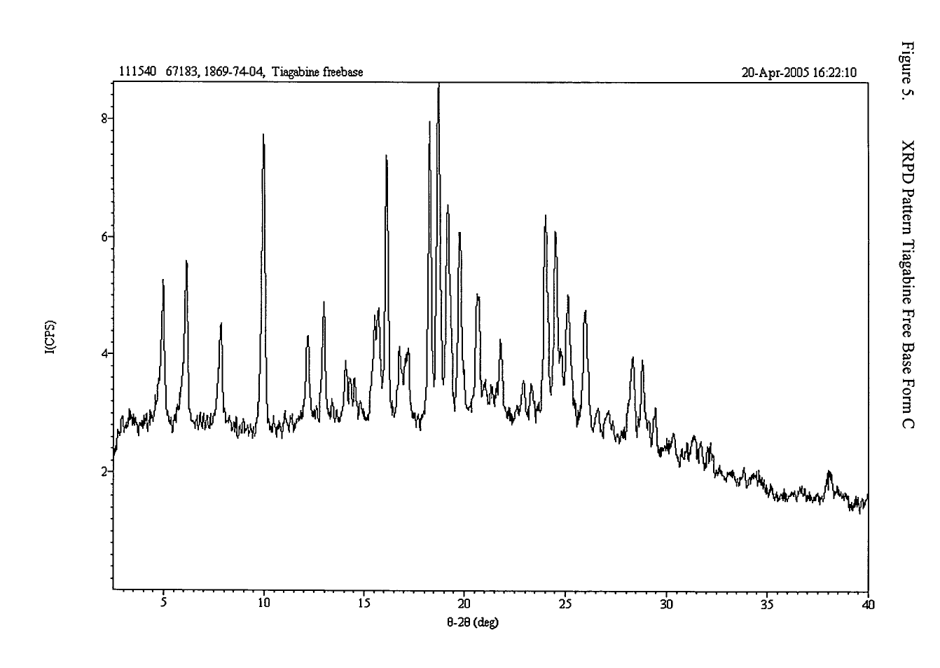

FIG. 5 depicts an XRPD pattern of tiagabine free base Form C.

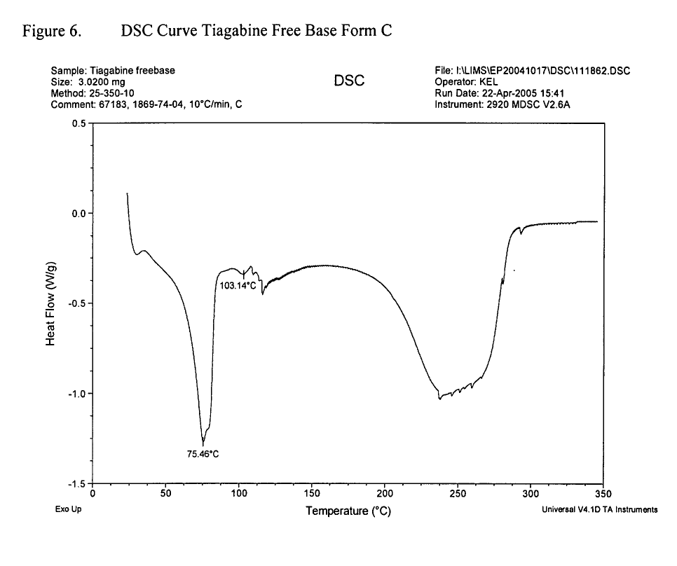

FIG. 6 depicts a DSC curve of tiagabine free base Form C.

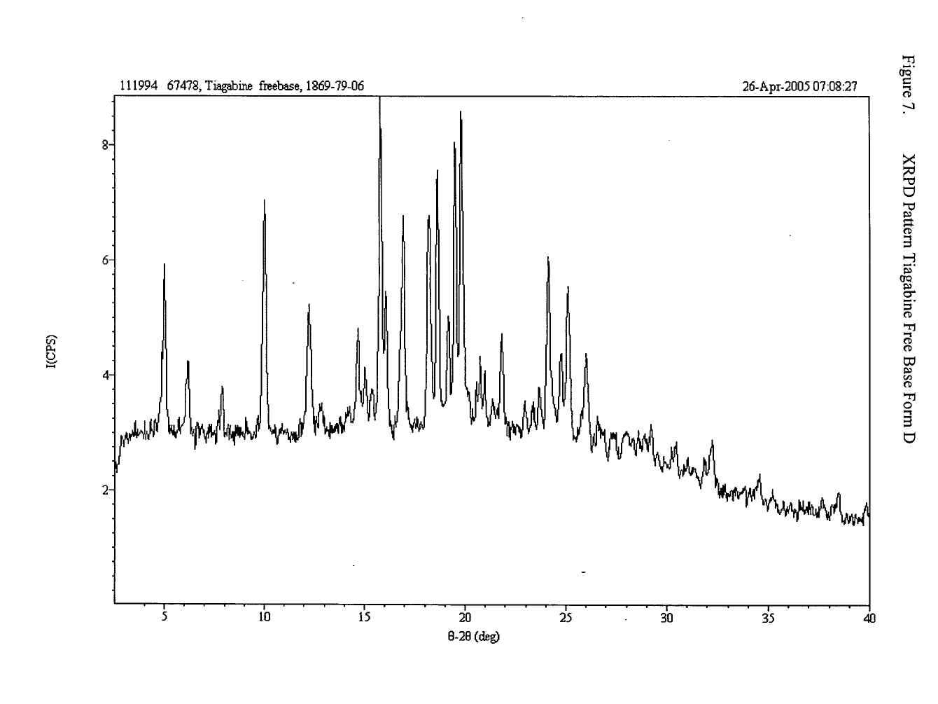

FIG. 7 depicts an XRPD pattern of tiagabine free base Form D.

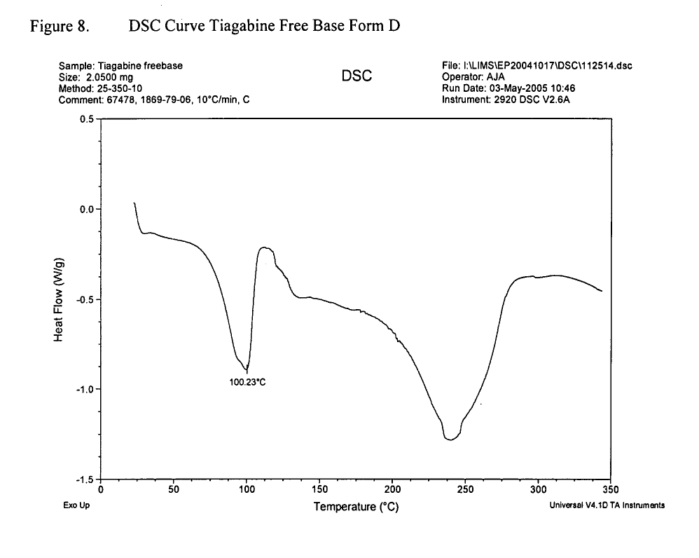

FIG. 8 depicts a DSC curve of tiagabine free base Form D.

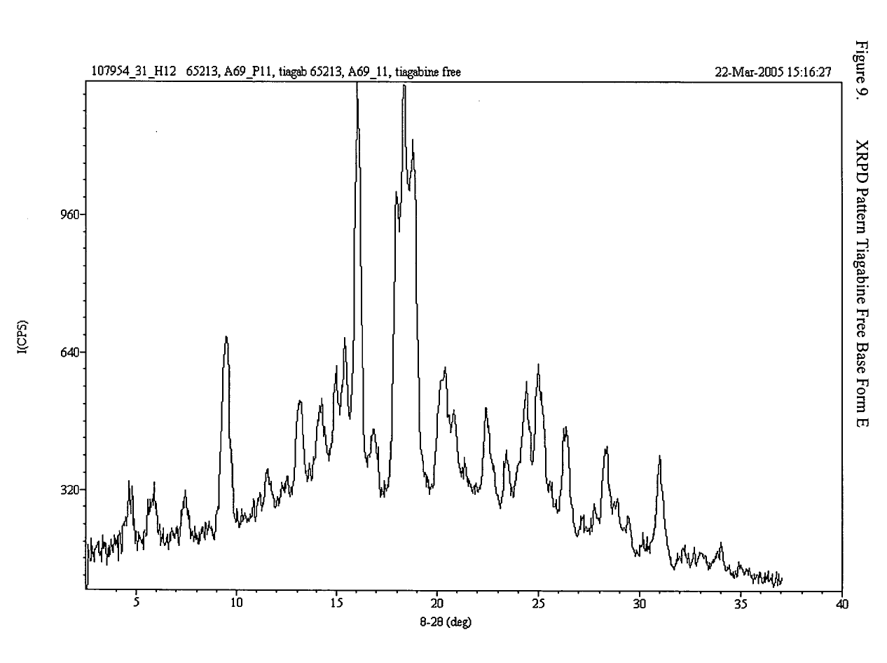

FIG. 9 depicts an XRPD pattern of tiagabine free base Form E.

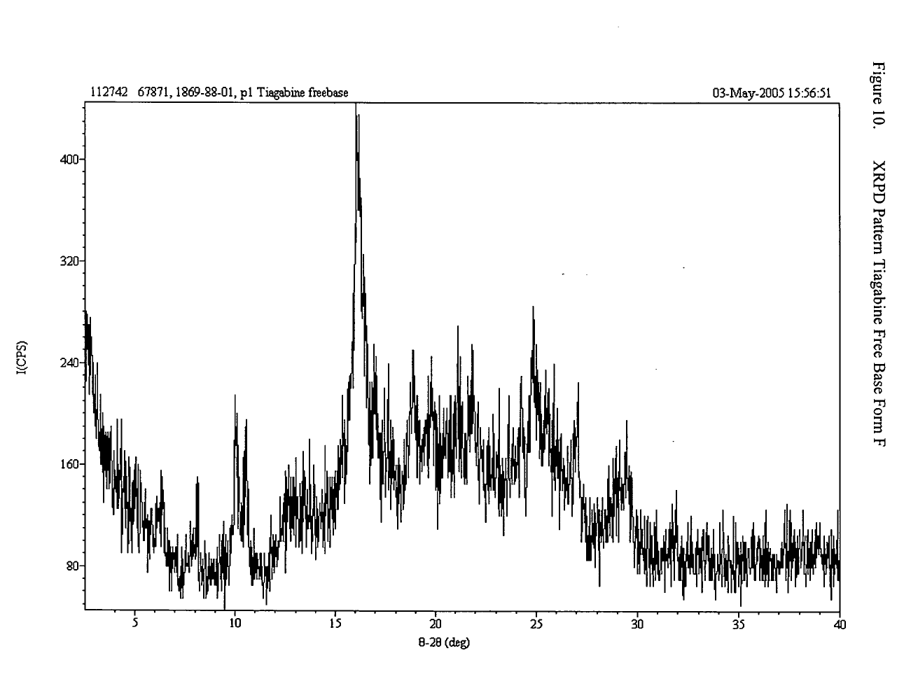

FIG. 10 depicts an XRPD pattern of tiagabine free base Form F.

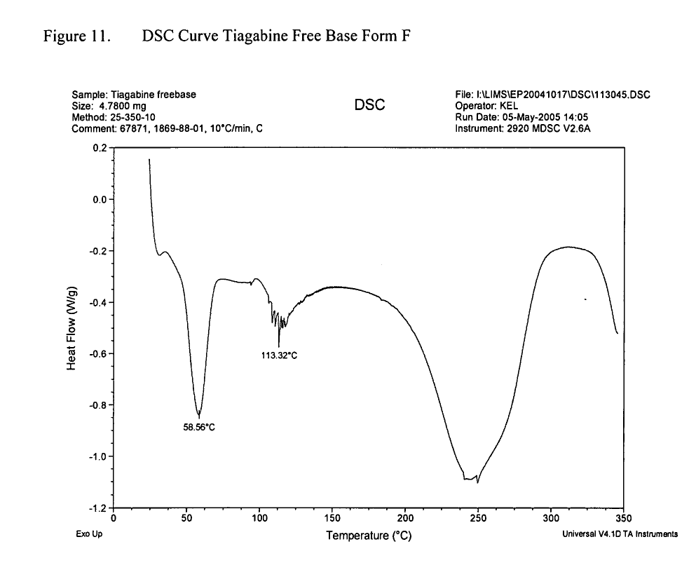

FIG. 11 depicts a DSC curve of tiagabine free base Form F.

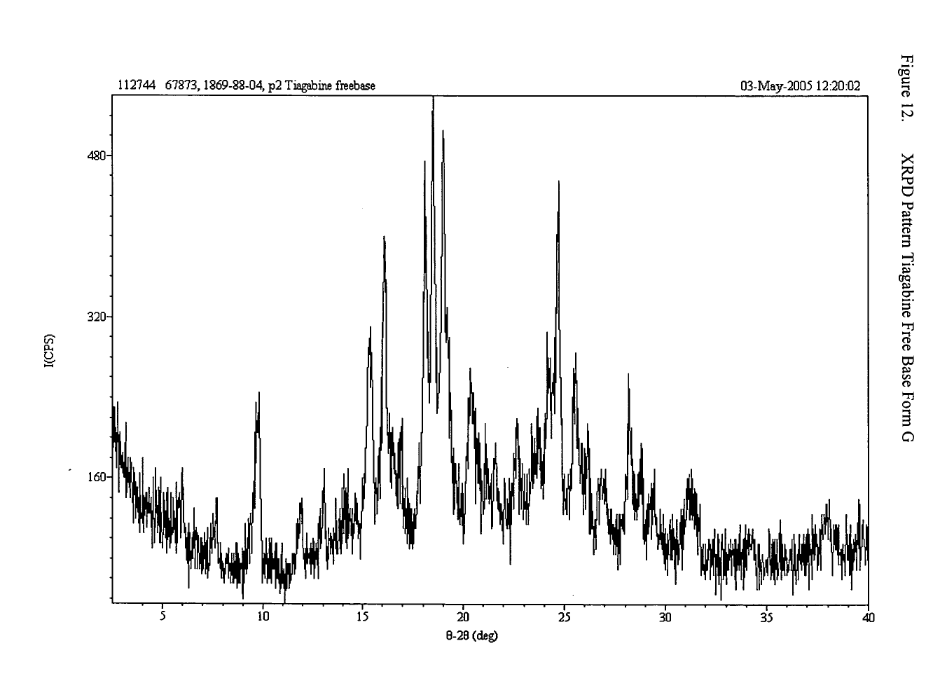

FIG. 12 depicts an XRPD pattern of tiagabine free base Form G.

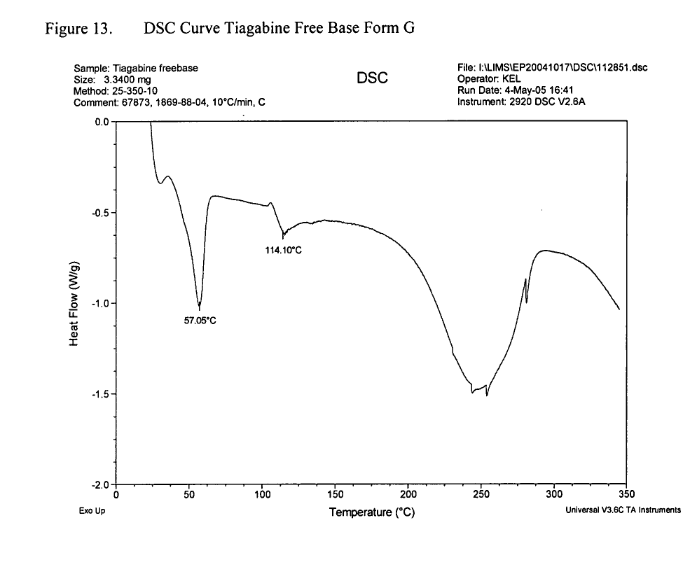

FIG. 13 depicts a DSC curve of tiagabine free base Form G.

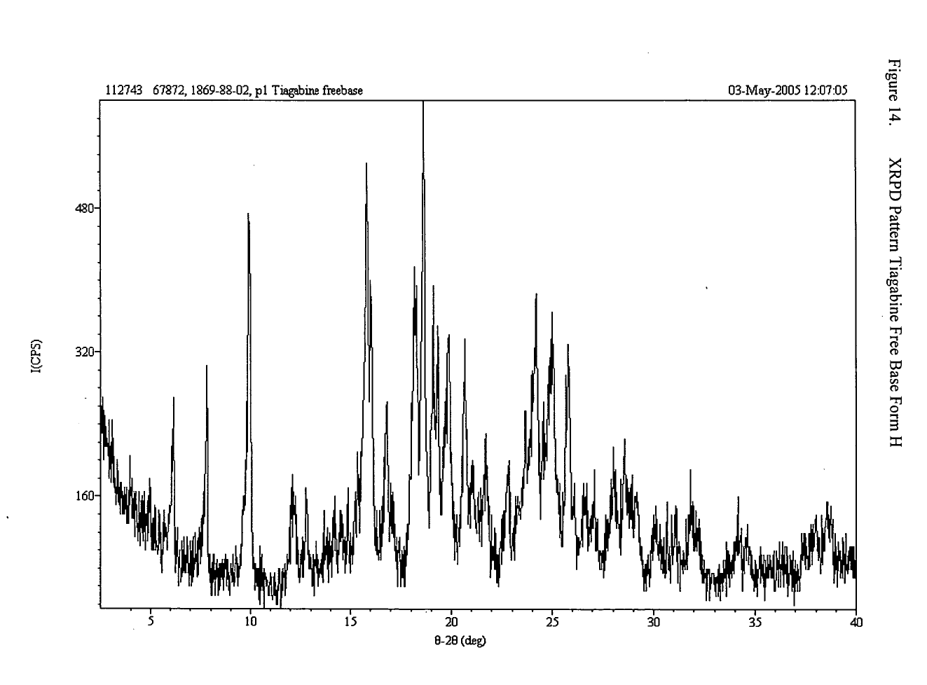

FIG. 14 depicts an XRPD pattern of tiagabine free base Form H.

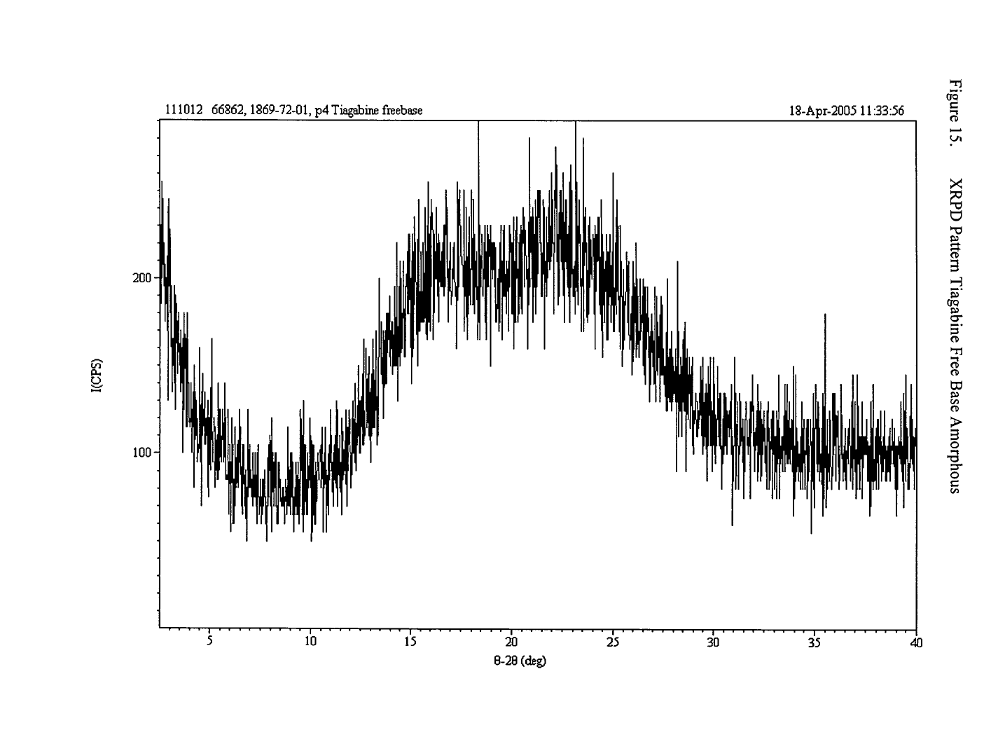

FIG. 15 depicts an XRPD pattern of tiagabine free base amorphous.

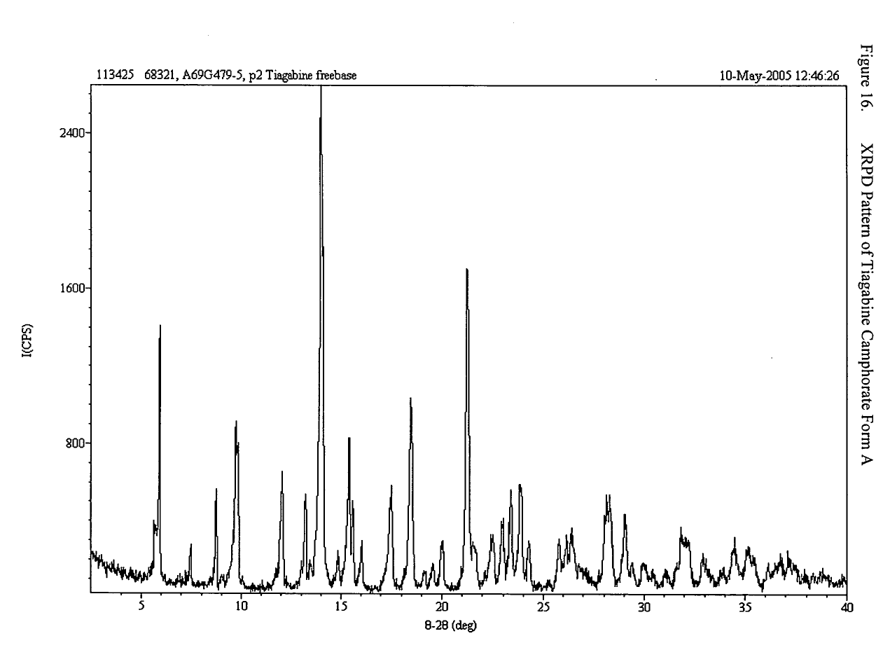

FIG. 16 depicts an XRPD pattern of tiagabine camphorate Form A.

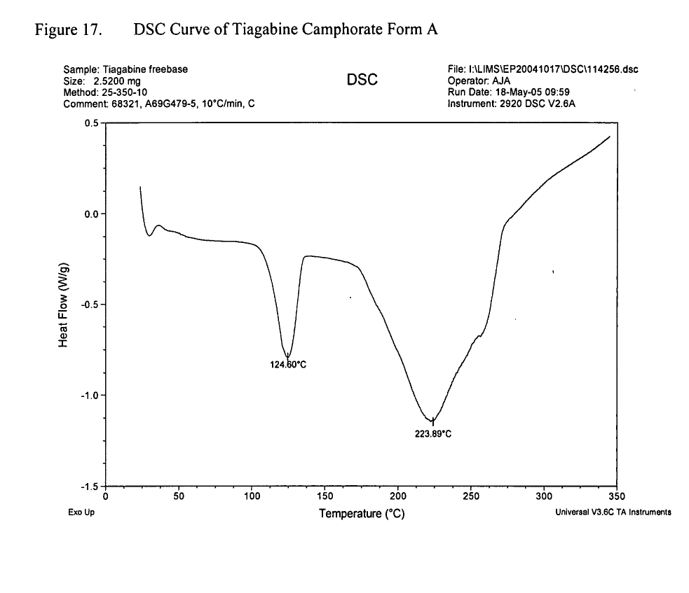

FIG. 17 depicts a DSC curve of tiagabine camphorate Form A.

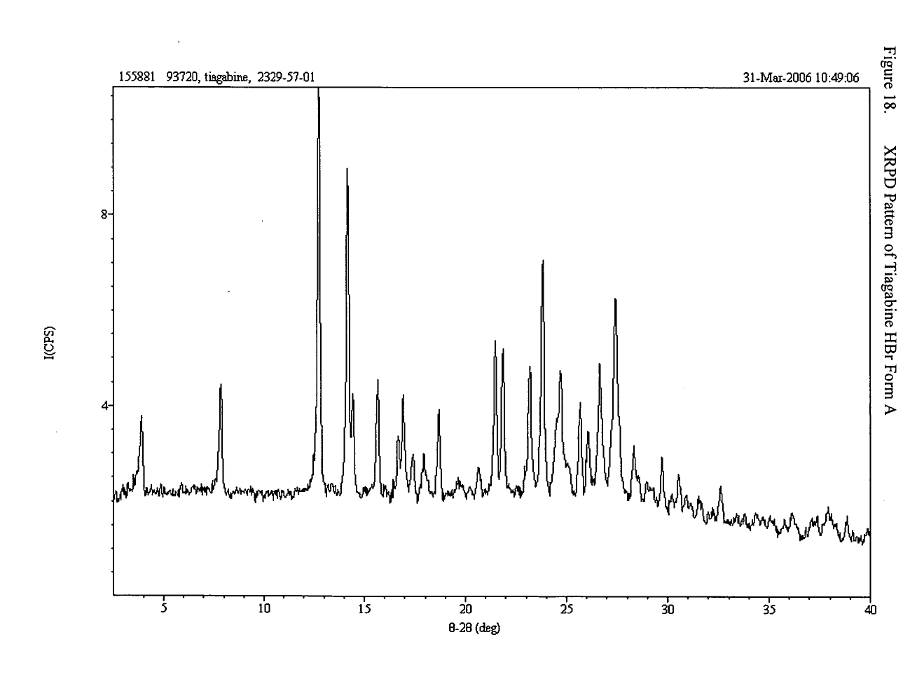

FIG. 18 depicts an XRPD pattern of tiagabine hydrobromide Form A.

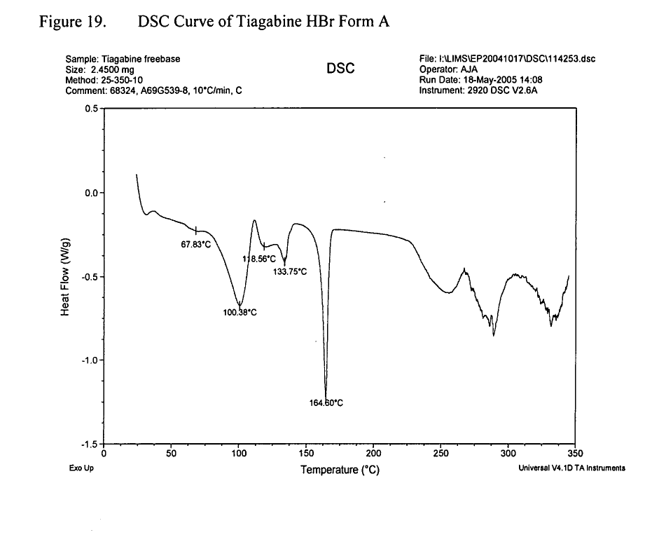

FIG. 19 depicts a DSC curve of tiagabine hydrobromide Form A.

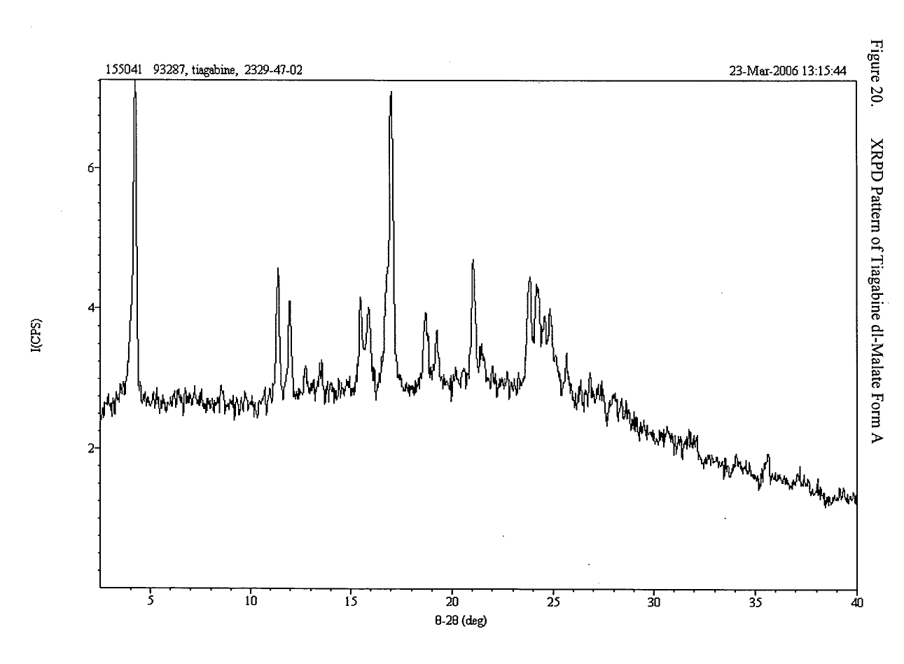

FIG. 20 depicts an XRPD pattern of tiagabine dl-malate Form A.

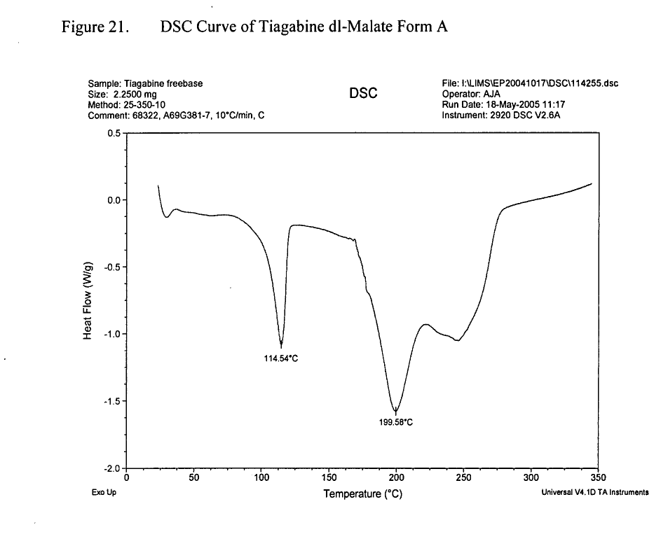

FIG. 21 depicts a DSC curve of tiagabine dl-malate Form A.

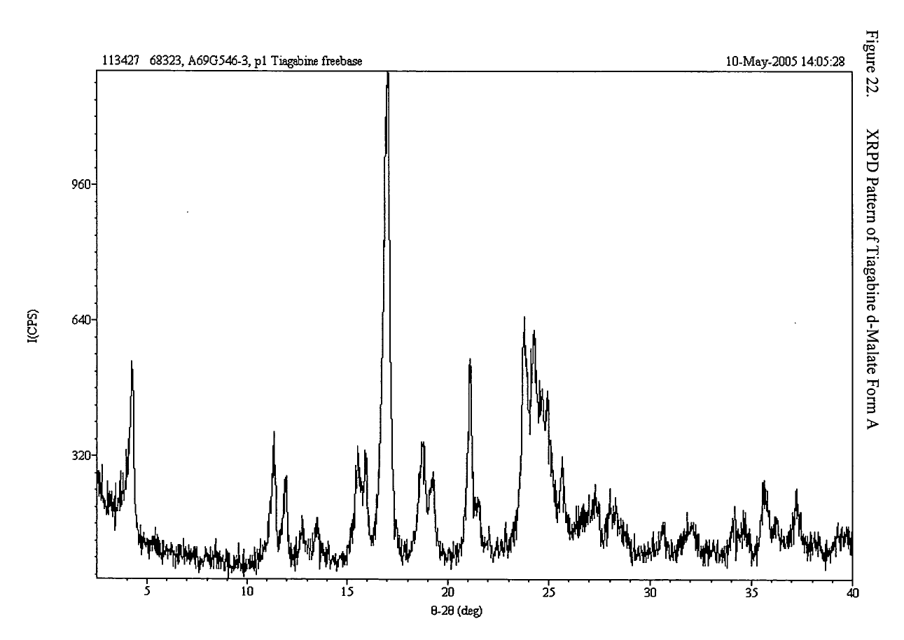

FIG. 22 depicts an XRPD pattern of tiagabine d-malate Form A.

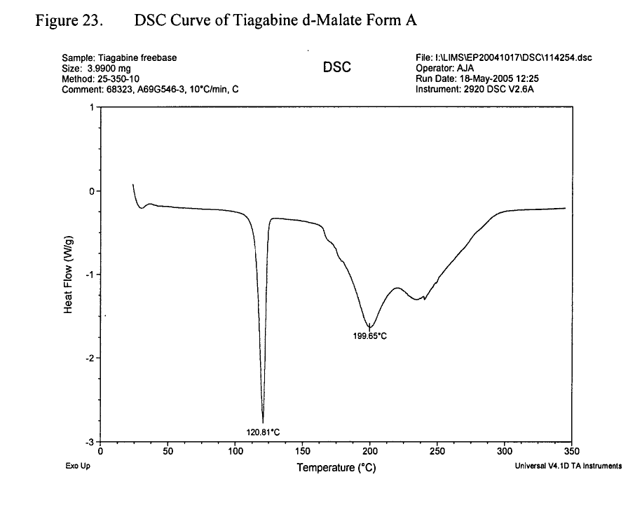

FIG. 23 depicts a DSC curve of tiagabine d-malate Form A.

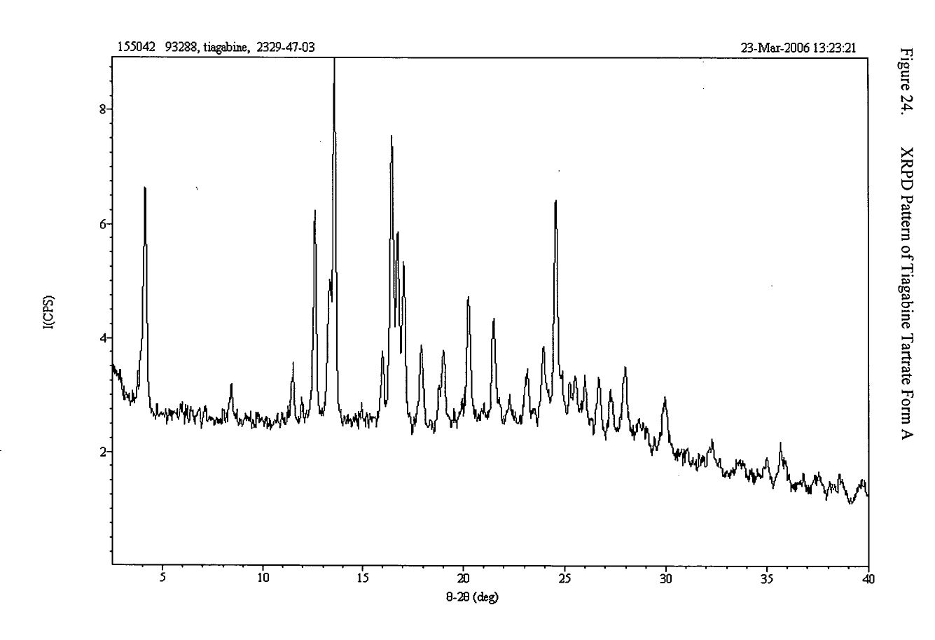

FIG. 24 depicts an XRPD pattern of tiagabine tartrate Form A.

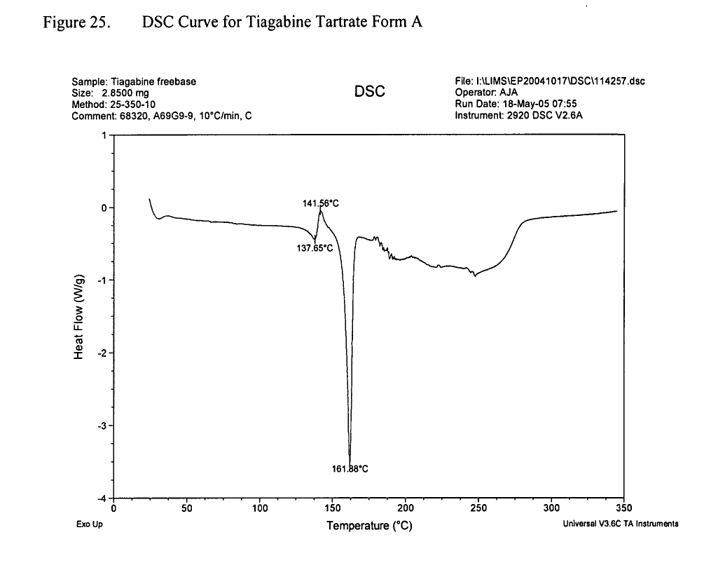

FIG. 25 depicts a DSC curve of tiagabine tartrate Form A.

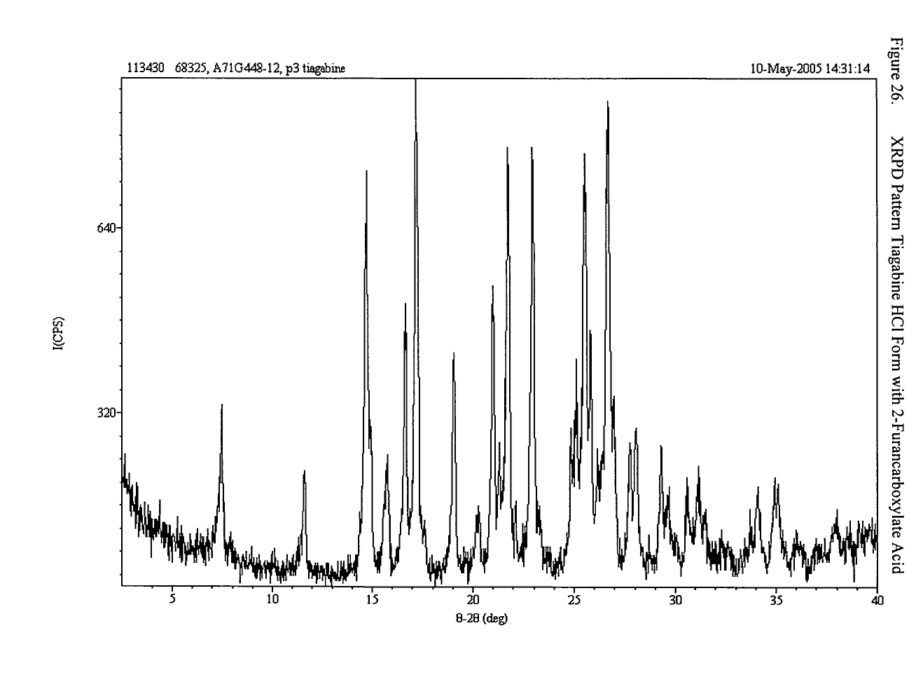

FIG. 26 depicts an XRPD pattern of tiagabine hydrochloride cocrystal with 2-furancarboxylic acid.

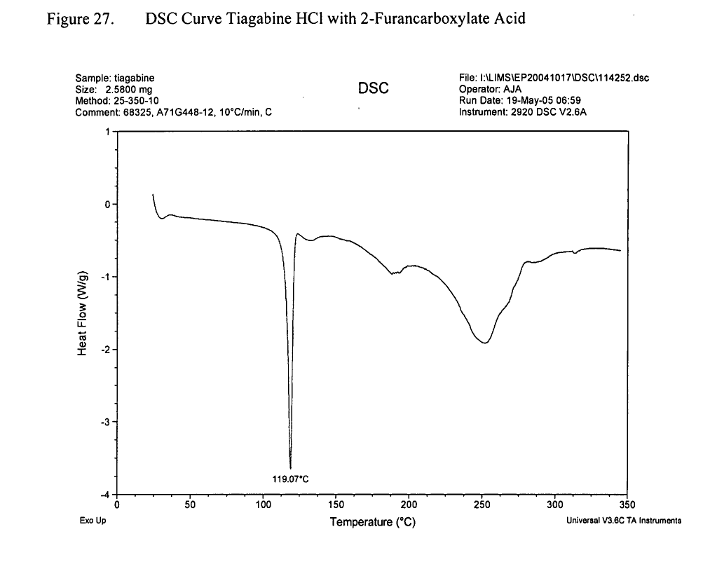

FIG. 27 depicts a DSC curve of tiagabine hydrochloride cocrystal with 2-furancarboxylic acid.

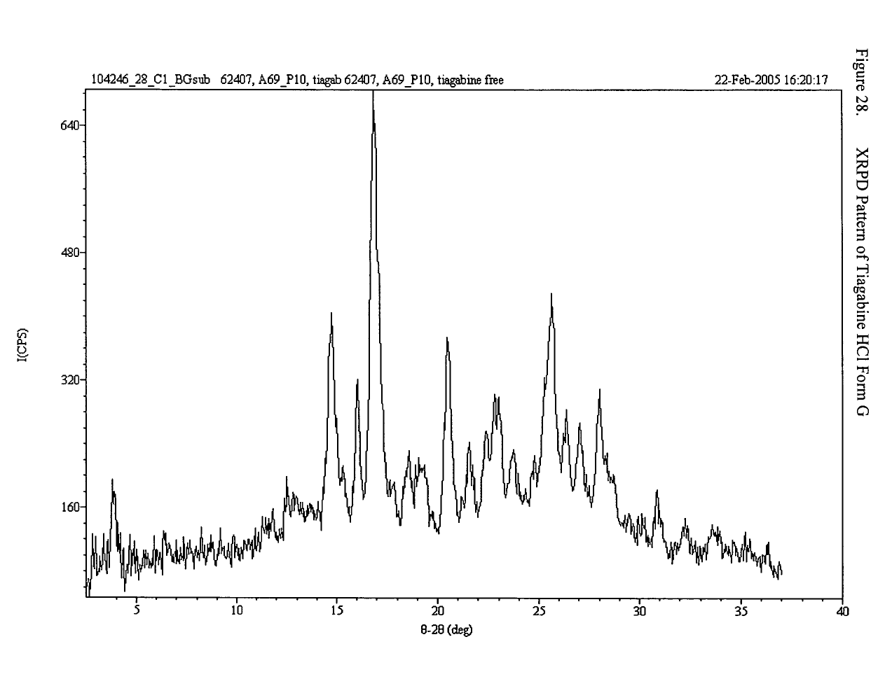

FIG. 28 depicts an XRPD pattern of tiagabine hydrochloride Form G.

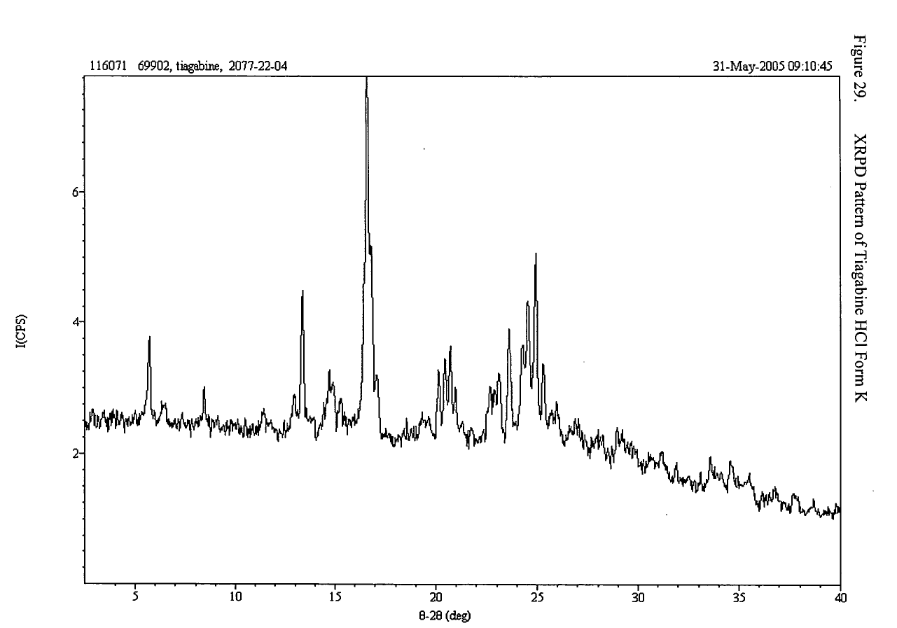

FIG. 29 depicts an XRPD pattern of tiagabine hydrochloride Form K.

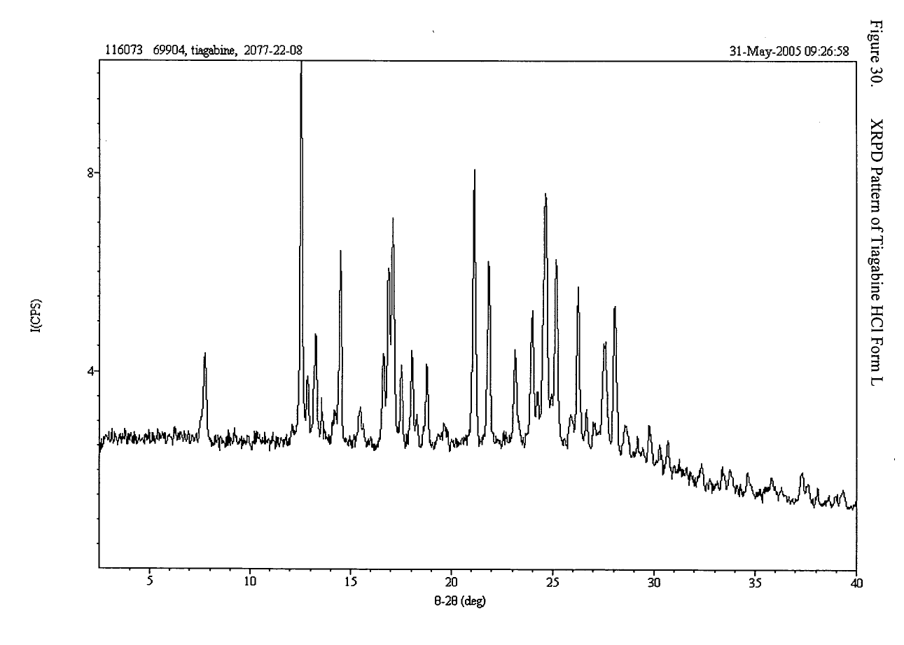

FIG. 30 depicts an XRPD pattern of tiagabine hydrochloride Form L.

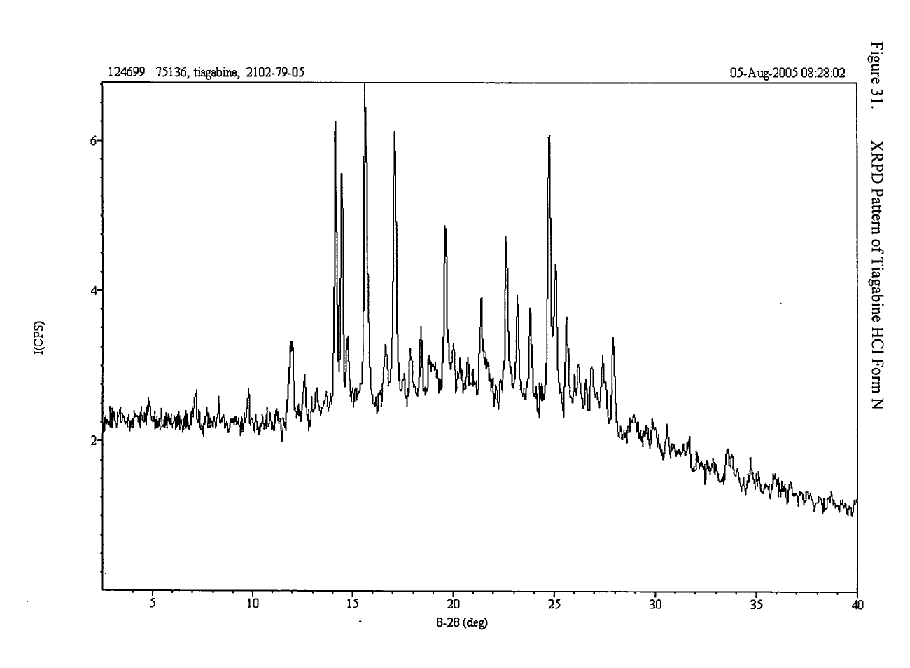

FIG. 31 depicts an XRPD pattern of tiagabine hydrochloride Form N.

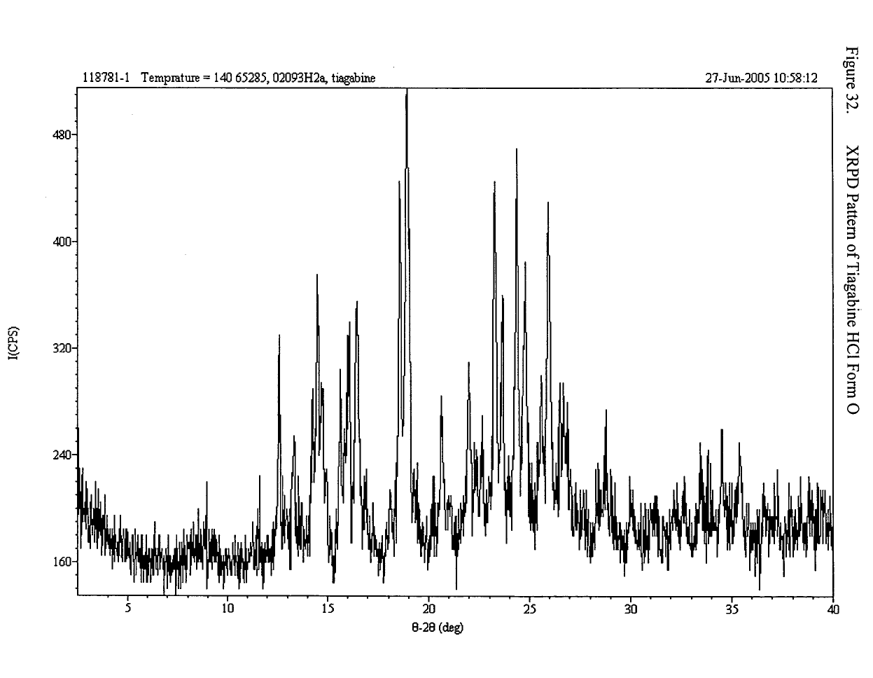

FIG. 32 depicts an XRPD pattern of tiagabine hydrochloride Form O.

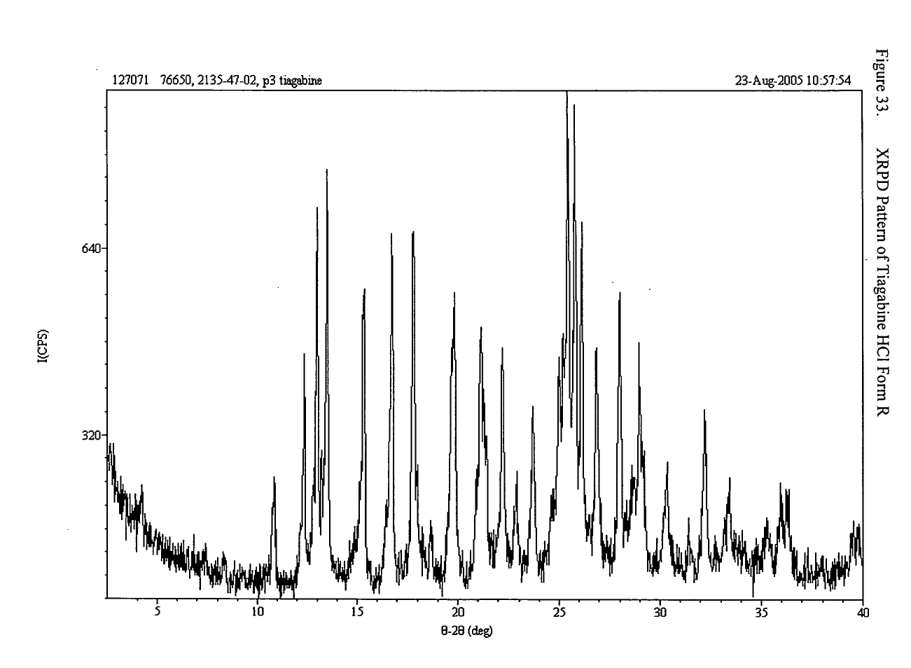

FIG. 33 depicts an XRPD pattern of tiagabine hydrochloride Form R.

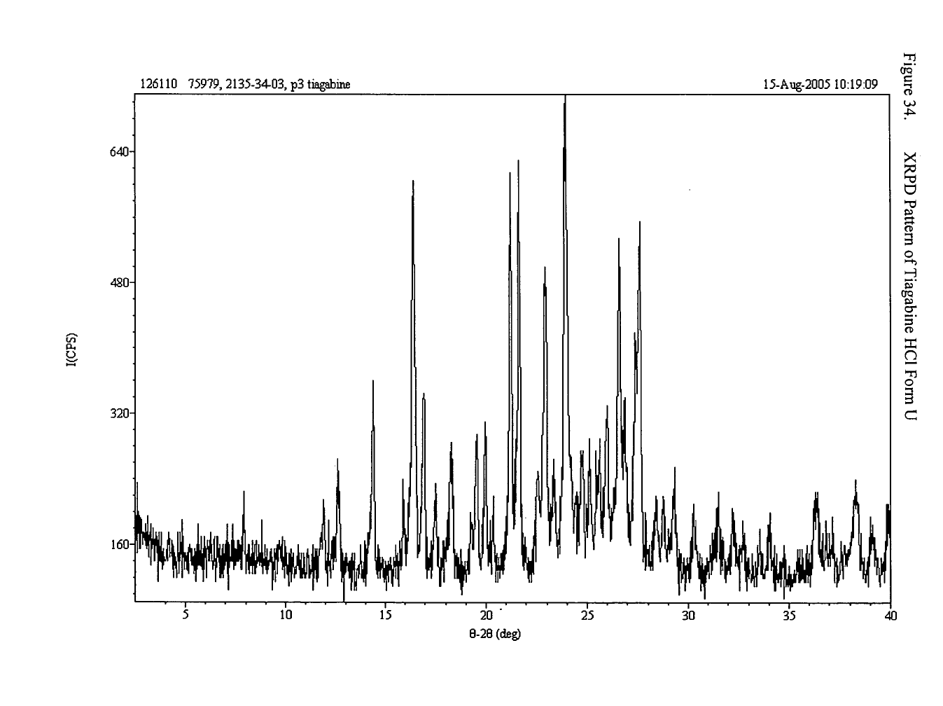

FIG. 34 depicts an XRPD pattern of tiagabine hydrochloride Form U.

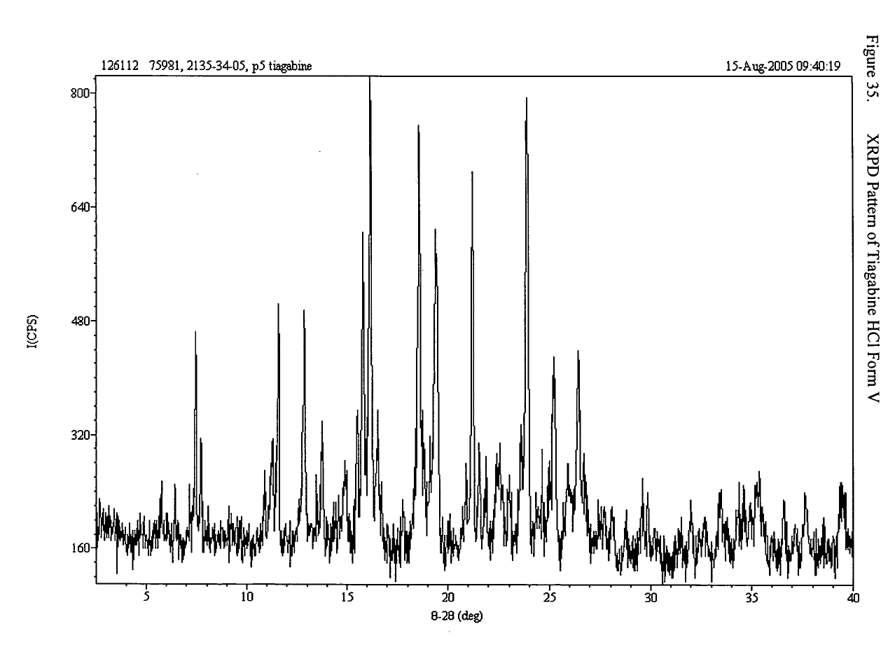

FIG. 35 depicts an XRPD pattern of tiagabine hydrochloride Form V.

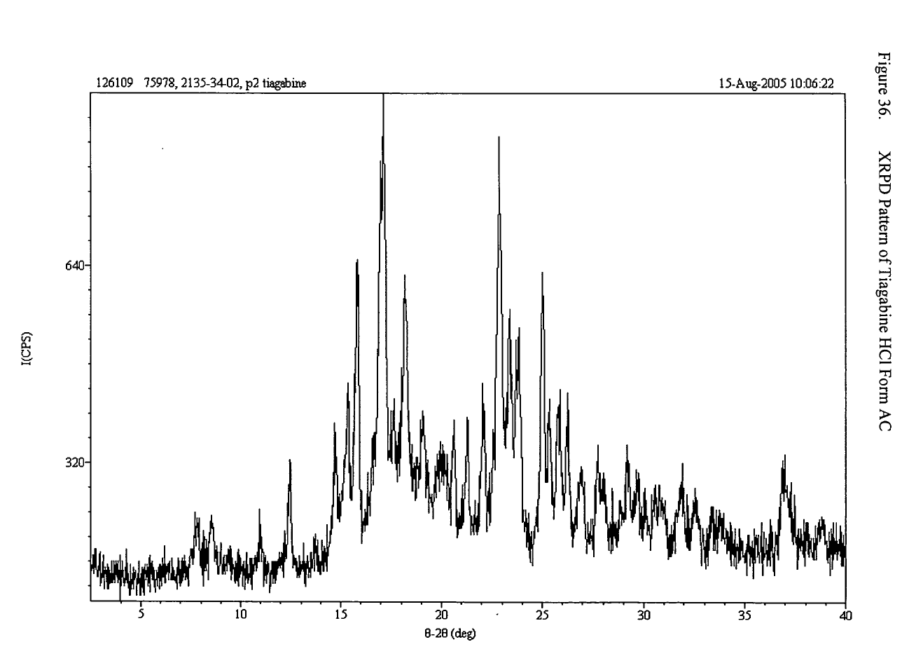

FIG. 36 depicts an XRPD pattern of tiagabine hydrochloride Form AC.

### DETAILED DESCRIPTION OF THE INVENTION

Definitions

“Crystalline form” refers to a solid chemical compound or mixture of compounds that provides a pattern of peaks when analyzed by x-ray powder diffraction; this includes polymorphs, solvates, hydrates, cocrystals, and desolvated solvates; “purity” refers to the relative quantity by weight of one component in a mixture (% w/w); “solution” refers to a mixture containing at least one solvent and at least one compound at least partially dissolved in the solvent.

Preparation and Characterization

The present invention provides 24 new tiagabine forms, including 22 new crystalline forms of tiagabine free base and salts thereof, an amorphous form of tiagabine free base, and a cocrystal form of tiagabine hydrochloride with 2-furancarboxylic acid. The 22 new crystalline forms include nine (9) new crystalline forms of tiagabine hydrochloride, eight (8) new crystalline forms of tiagabine free base, one (1) new crystalline form of tiagabine camphorate, one (1) new crystalline form of tiagabine hydrobromide, one (1) new crystalline form of tiagabine dl-malate, one (1) new crystalline form of tiagabine d-malate, and one (1) new crystalline form of tiagabine tartrate.

Tiagabine Free Base Form A

Tiagabine free base Form A may be prepared by crystallizing tiagabine free base from ethanol. Tiagabine free base Form A also may be prepared by slurrying tiagabine free base in a mixture of hexane, diisopropylether, and ethanol. Preferably, the hexane, diisopropylether, and ethanol are present in the slurry mixture in a ratio of about 100:20:3 (v/v/v).

The XRPD pattern of tiagabine free base Form A contains peaks at 6.5, 8.1, 12.6, 17.4, 19.0, 19.5, 22.9, 25.8, and 27.2±0.2 degrees 2θ. A representative XRPD pattern of tiagabine free base Form A is presented in FIG. 1.

Preferably, the tiagabine free base Form A of the present invention has a purity of at least about 50% (w/w). More preferably, the tiagabine free base Form A has a purity of at least about 70% (w/w). More preferably, the tiagabine free base Form A has a purity of at least about 90% (w/w).

Tiagabine Free Base Form B

Tiagabine free base Form B may be prepared by drying tiagabine free base Form A under vacuum. Tiagabine free base Form B also may be prepared by crystallizing tiagabine from a mixture of tetrahydrofuran and isopropanol. Tiagabine free base Form B also may be prepared by crystallizing tiagabine from ethanol.

The XRPD pattern of tiagabine free base Form B contains peaks at 15.0, 15.4, 17.3, 21.3, 22.5, and 24.8±0.2 degrees 2θ. A representative XRPD pattern of tiagabine free base Form B is presented in FIG. 3.

Preferably, the tiagabine free base Form B of the present invention has a purity of at least about 50% (w/w). More preferably, the tiagabine free base Form B has a purity of at least about 70% (w/w). More preferably, the tiagabine free base Form B has a purity of at least about 90% (w/w).

Tiagabine Free Base Form C

Tiagabine free base Form C may be prepared by crystallizing (e.g., slurrying) tiagabine free base from isopropanol. Tiagabine free base Form C also may be prepared by crystallizing tiagabine free base from acetonitrile. Tiagabine free base Form C also may be prepared by crystallizing tiagabine free base from ethanol. Tiagabine free base Form C also may be prepared by crystallizing tiagabine free base from isopropanol, optionally in admixture with cyclohexane. Tiagabine free base Form C also may be prepared by crystallizing tiagabine free base from a mixture of tetrahydrofuran and isopropanol, optionally in admixture with acetonitrile

Tiagabine free base Form C also may be prepared by crystallizing tiagabine free base from a mixture of methyl ethyl ketone and 2,2,2-trifluoroethanol, optionally in admixture with acetonitrile and/or isopropyl ether. Preferably, tiagabine free base Form C is prepared by adding acetonitrile to a mixture of methyl ethyl ketone and 2,2,2-trifluoroethanol. Preferably, tiagabine free base Form C is prepared by crystallizing tiagabine free base from a 1:1 (v/v) mixture of methyl ethyl ketone and 2,2,2-trifluoroethanol.

The XRPD pattern of tiagabine free base Form C contains peaks at 4.9, 6.1, 7.8, 9.9, 12.2, and 12.9±0.2 degrees 2θ. A representative XRPD pattern of tiagabine free base Form C is presented in FIG. 5.

Preferably, the tiagabine free base Form C of the present invention has a purity of at least about 50% (w/w). More preferably, the tiagabine free base Form C has a purity of at least about 70% (w/w). More preferably, the tiagabine free base Form C has a purity of at least about 90% (w/w).

Tiagabine Free Base Form D

Tiagabine free base Form D may be prepared by crystallizing tiagabine free base from a mixture of 2,2,2-trifluoroethanol and methyl ethyl ketone. Preferably, tiagabine free base Form D is prepared by crystallizing tiagabine free base from a mixture of 2,2,2-trifluoroethanol and methyl ethyl ketone at a ratio of 1:1 (v/v). Tiagabine free base Form D also may be prepared by crystallizing tiagabine free base from 2-propyl ether.

The XRPD pattern of tiagabine free base Form D contains peaks at 5.7, 6. 1, 10.0, 12.2, 15.8, and 16.9±0.2 degrees 2θ. A representative XRPD pattern of tiagabine free base Form D is presented in FIG. 7.

Preferably, the tiagabine free base Form D of the present invention has a purity of at least about 50% (w/w). More preferably, the tiagabine free base Form D has a purity of at least about 70% (w/w). More preferably, the tiagabine free base Form D has a purity of at least about 90% (w/w).

Tiagabine Free Base Form E

Tiagabine free base Form E may be prepared by crystallizing tiagabine free base from a mixture of propionitrile and t-butyl alcohol. Preferably, tiagabine free base Form E is prepared by crystallizing tiagabine free base from a mixture of propionitrile and t-butyl alcohol at a ratio of 1:1 (v/v). Tiagabine free base Form E also may be prepared by crystallizing tiagabine free base from a mixture of 2,2,2-trifluoroethanol and methyl ethyl ketone at a ratio of 1:1 (v/v). Tiagabine free base Form E also may be prepared by crystallizing tiagabine free base from acetonitrile. Tiagabine free base Form E also may be prepared by crystallizing tiagabine free base from a mixture of 2,2,2-trifluoroethanol, methyl ethyl ketone, and propyl ether.

The XRPD pattern of tiagabine free base Form E contains peaks at 9.5, 13.1, 14.3, 16.1, 18.7, and 22.5±0.2 degrees 2θ. A representative XRPD pattern of tiagabine free base Form E is presented in FIG. 9.

Preferably, the tiagabine free base Form E of the present invention has a purity of at least about 50% (w/w). More preferably, the tiagabine free base Form E has a purity of at least about 70% (w/w). More preferably, the tiagabine free base Form E has a purity of at least about 90% (w/w).

Tiagabine Free Base Form F

Tiagabine free base Form F may be prepared by crystallizing tiagabine free base from a mixture of methanol and 2-propyl ether. Preferably, tiagabine free base Form F is prepared by crystallizing tiagabine free base from a mixture of methanol and 2-propyl ether at a ratio of 1:2 (v/v).

The XRPD pattern of tiagabine free base Form F contains peaks at 6.3, 8.0, 10.0, 10.5, 16.2, 21.1, and 21.8±0.2 degrees 2θ. A representative XRPD pattern of tiagabine free base Form F is presented in FIG. 10.

Preferably, the tiagabine free base Form F of the present invention has a purity of at least about 50% (w/w). More preferably, the tiagabine free base Form F has a purity of at least about 70% (w/w). More preferably, the tiagabine free base Form F has a purity of at least about 90% (w/w).

Tiagabine Free Base Form G

Tiagabine free base Form G may be prepared by crystallizing tiagabine free base from 2-butanol.

The XRPD pattern of tiagabine free base Form G contains peaks at 6.0, 7.6, 9.7, 15.4, 16.1, 18.1, 18.5, 19.0, and 24.7±0.2 degrees 2θ. A representative XRPD pattern of tiagabine free base Form G is presented in FIG. 12.

Preferably, the tiagabine free base Form G of the present invention has a purity of at least about 50% (w/w). More preferably, the tiagabine free base Form G has a purity of at least about 70% (w/w). More preferably, the tiagabine free base Form G has a purity of at least about 90% (w/w).

Tiagabine Free Base Form H

Tiagabine free base Form H may be prepared by crystallizing tiagabine free base from 1-propanol.

The XRPD pattern of tiagabine free base Form H contains peaks at 15.8, 16.8, and 20.7±0.2 degrees 2θ. A representative XRPD pattern of tiagabine free base Form H is presented in FIG. 14.

Preferably, the tiagabine free base Form H of the present invention has a purity of at least about 50% (w/w). More preferably, the tiagabine free base Form H has a purity of at least about 70% (w/w). More preferably, the tiagabine free base Form H has a purity of at least about 90% (w/w).

Tiagabine Free Base Amorphous

Tiagabine free base amorphous may be prepared by drying a sample of tiagabine free base Form A. Tiagabine free base amorphous also may be prepared by evaporating a 1,4-dioxane solution of tiagabine free base. Tiagabine free base amorphous also may be prepared by evaporating an isopropanol solution of tiagabine free base. Tiagabine free base amorphous also may be prepared by adding propyl ether to a solution of tiagabine free base in 1,4-dioxane. Tiagabine free base amorphous also may be prepared by precipitating tiagabine free base from a mixture of acetonitrile and dichloromethane.

A representative XRPD pattern of tiagabine free base amorphous is presented in FIG. 15.

Preferably, the tiagabine free base amorphous of the present invention has a purity of at least about 50% (w/w). More preferably, the tiagabine free base amorphous has a purity of at least about 70% (w/w). More preferably, the tiagabine free base amorphous has a purity of at least about 90% (w/w).

Tiagabine Camphorate Form A

Tiagabine camphorate Form A may be prepared by the steps of:
(a) preparing a solution of tiagabine free base and (+)-camphoric acid in methanol, and
(b) crystallizing tiagabine camphorate Form A from the solution.

Preferably, the solution further comprises acetonitrile. Preferably, the solution comprises methanol and acetonitrile in a ratio of about 2:1 to about 1:2 (v/v). More preferably, the solution comprises methanol and acetonitrile in a ratio of about 1:1.5 (v/v).

Preferably, the solution further comprises acetonitrile and ethyl acetate. Preferably, the solution comprises methanol, acetonitrile, and ethyl acetate at a ratio of about 1:4:1 (v/v/v).

The XRPD pattern of tiagabine camphorate Form A contains peaks at 5.9, 9.8, 12.0, 14.0, 15.4, 18.4, and 21.2±0.2 degrees 2θ. A representative XRPD pattern of tiagabine camphorate Form A is presented in FIG. 16.

Preferably, the tiagabine camphorate Form A of the present invention has a purity of at least about 50% (w/w). More preferably, the tiagabine camphorate Form A has a purity of at least about 70% (w/w). More preferably, the tiagabine camphorate Form A has a purity of at least about 90% (w/w).

Tiagabine Hydrobromide Form A

Tiagabine hydrobromide Form A may be prepared by the steps of:
(a) preparing a solution of tiagabine free base and hydrobromic acid in a mixture of ethyl acetate and acetonitrile; and
(b) crystallizing tiagabine hydrobromide Form A from the solution.

Preferably, the solution contains ethyl acetate and acetonitrile at a ratio of about 1:2 to about 5:1 (v/v). More preferably, the solution contains ethyl acetate and acetonitrile at a ratio of about 1:1 to about 2:1 (v/v).

Preferably, the solution further comprises 2-propyl ether.

Tiagabine hydrobromide Form A also may be prepared by the steps of:
(a) layering a solution of hydrobromic acid in diisopropyl ether onto a solution of tiagabine free base in a mixture of ethyl acetate and acetonitrile, and
(b) crystallizing tiagabine hydrobromide Form A from the layered solutions.

Preferably, the mixture in step (a) contains ethyl acetate and acetonitrile at a ratio of about 3:1 (v/v).

The XRPD pattern of tiagabine hydrobromide Form A contains peaks at 3.9, 7.8, 12.8, 14.2, 14.4, 15.7, 21.5, and 21.8±0.2 degrees 2θ. A representative XRPD pattern of tiagabine hydrobromide Form A is presented in FIG. 18.

Preferably, the tiagabine hydrobromide Form A of the present invention has a purity of at least about 50% (w/w). More preferably, the tiagabine hydrobromide Form A has a purity of at least about 70% (w/w). More preferably, the tiagabine hydrobromide Form A has a purity of at least about 90% (w/w).

Tiagabine dl-Malate Form A

Tiagabine dl-malate Form A may be prepared by the steps of:
(a) preparing a solution of tiagabine free base and dl-malic acid in a mixture of ethyl acetate, acetonitrile and methanol, and
(b) crystallizing tiagabine dl-malate Form A from the solution.

Tiagabine dl-malate Form A also may be prepared by the steps of:
(a) preparing a solution of tiagabine free base and dl-malic acid in a mixture of tetrahydrofuran and 2-propanol; and
(b) crystallizing tiagabine dl-malate Form A from the solution.

Preferably, the solution contains tetrahydrofuran and 2-propanol at a ratio of about 0.5:1 to about 5:1 (v/v). More preferably, the solution contains tetrahydrofuran and 2-propanol at a ratio of about 2:1 (v/v).

The XRPD pattern of tiagabine dl-malate Form A contains peaks at 4.2, 11.3, 11.9, 15.5, 15.9, 18.7, and 19.2±0.2 degrees 2θ. A representative XRPD pattern of tiagabine dl-malate Form A is presented in FIG. 20.

Preferably, the tiagabine dl-malate Form A of the present invention has a purity of at least about 50% (w/w). More preferably, the tiagabine dl-malate Form A has a purity of at least about 70% (w/w). More preferably, the tiagabine dl-malate Form A has a purity of at least about 90% (w/w).

Tiagabine d-Malate Form A

Tiagabine d-malate Form A may be prepared by the steps of:
(a) preparing a solution of tiagabine free base and d-malic acid in a mixture of ethyl acetate and acetonitrile, and
(b) crystallizing tiagabine d-malate Form A from the solution.

Preferably, the solution contains ethyl acetate and acetonitrile at a ratio of about 1:1 to about 5:1 (v/v/v). More preferably, the solution contains ethyl acetate and acetonitrile at a ratio of about 3:1 (v/v/v).

Preferably, the solution further comprises methanol.

Preferably, the process for preparing tiagabine d-malate Form A further comprises the step of:
(c) slurrying the crystallized tiagabine d-malate Form A in ether.

The XRPD pattern of tiagabine d-malate Form A contains peaks at 4.2, 11.3, 11.9, 15.9, 17.0, 18.7, 21.1, and 23.8±0.2 degrees 2θ. A representative XRPD pattern of tiagabine d-malate Form A is presented in FIG. 22.

Preferably, the tiagabine d-malate Form A of the present invention has a purity of at least about 50% (w/w). More preferably, the tiagabine d-malate Form A has a purity of at least about 70% (w/w). More preferably, the tiagabine d-malate Form A has a purity of at least about 90% (w/w).

Tiagabine Tartrate Form A

Tiagabine tartrate Form A may be prepared by the steps of:
(a) preparing a solution of tiagabine free base and L-(+)-tartaric acid in a mixture of methanol and acetonitrile, and
(b) crystallizing tiagabine tartrate Form A from the solution.

Preferably, the solution contains methanol and acetonitrile at a ratio of about 0.5:1 to about 5:1 (v/v). More preferably, the solution contains methanol and acetonitrile at a ratio of about 1.5:1 (v/v).

Preferably, the solution further comprises ethyl acetate. Preferably, the solution contains methanol, acetonitrile, and ethyl acetate at a ratio of about 1:1:1 to about 1:5:10 (v/v/v). More preferably, the solution contains methanol, acetonitrile, and ethyl acetate at a ratio of about 1:2:2.5 (v/v/v).

Tiagabine tartrate Form A also may be prepared by the steps of:
(a) preparing a solution of tiagabine free base and L-(+)-tartaric acid in a mixture of acetone and ethyl acetate; and
(b) crystallizing tiagabine tartrate Form A from the solution.

Preferably, the solution contains acetone and ethyl acetate at a ratio of about 1:5 to about 5:1 (v/v). More preferably, the solution contains acetone and ethyl acetate at a ratio of about 1:1 (v/v).

Tiagabine tartrate Form A also may be prepared by the steps of:
(a) preparing a solution of tiagabine free base and L-(+)-tartaric acid in a mixture of tetrahydrofuran and 2-propanol; and
(b) crystallizing tiagabine tartrate Form A from the solution.

Preferably, the solution contains tetrahydrofuran and 2-propanol at a ratio of about 1:2 to about 10:1 (v/v). More preferably, the solution contains tetrahydrofuran and 2-propanol at a ratio of about 2:1 (v/v).

The XRPD pattern of tiagabine tartrate Form A contains peaks at 4.1, 11.5, 12.6, 13.6, 16.5, 16.7, 21.5, and 24.6±0.2 degrees 2θ. A representative XRPD pattern of tiagabine tartrate Form A is presented in FIG. 24.

Preferably, the tiagabine tartrate Form A of the present invention has a purity of at least about 50% (w/w). More preferably, the tiagabine tartrate Form A has a purity of at least about 70% (w/w). More preferably, the tiagabine tartrate Form A has a purity of at least about 90% (w/w).

Crystalline Form A of Tiagabine Hydrochloride Cocrystal with 2-Furancarboxylic Acid

Crystalline Form A of tiagabine hydrochloride cocrystal with 2-furancarboxylic acid may be prepared by the steps of:
(a) preparing a mixture of tiagabine hydrochloride and 2-furancarboxylic acid; and
(b) grinding the mixture to form crystalline Form A of tiagabine hydrochloride cocrystal with 2-furancarboxylic acid.

Preferably, the mixture further comprises methanol.

Preferably, the tiagabine hydrochloride is tiagabine hydrochloride monohydrate.

Preferably, the grinding step (b) is performed using a ball mill.

The XRPD pattern of crystalline Form A of tiagabine hydrochloride cocrystal with 2-furancarboxylic acid contains peaks at 7.5, 11.6, 14.7, 17.2, 21.7, 22.9 and 26.6±0.2 degrees 2θ. A representative XRPD pattern of crystalline Form A of tiagabine hydrochloride cocrystal with 2-furancarboxylic acid is presented in FIG. 26.

Preferably, the crystalline Form A of tiagabine hydrochloride cocrystal with 2-furancarboxylic acid of the present invention has a purity of at least about 50% (w/w). More preferably, the crystalline Form A of tiagabine hydrochloride cocrystal with 2-furancarboxylic acid has a purity of at least about 70% (w/w). More preferably, the crystalline Form A of tiagabine hydrochloride cocrystal with 2-furancarboxylic acid has a purity of at least about 90% (w/w).

Tiagabine Hydrochloride Form G

Tiagabine hydrochloride Form G may be prepared by crystallizing tiagabine hydrochloride from chloroform. Tiagabine hydrochloride Form G also may be prepared by crystallizing tiagabine hydrochloride from a mixture of chloroform, methanol, and cyclohexane.

The XRPD pattern of tiagabine hydrochloride Form G contains peaks at 3.9, 14.7, 16.0, 16.9, 20.5, 25.5, and 28.1±0.2 degrees 2θ. A representative XRPD pattern of tiagabine hydrochloride Form G is presented in FIG. 28.

Preferably, the tiagabine hydrochloride Form G of the present invention has a purity of at least about 50% (w/w). More preferably, the tiagabine hydrochloride Form G has a purity of at least about 70% (w/w). More preferably, the tiagabine hydrochloride Form G has a purity of at least about 90% (w/w).

Tiagabine Hydrochloride Form K

Tiagabine hydrochloride Form K may be prepared by crystallizing tiagabine hydrochloride from chloroform, optionally in admixture with heptane.

The XRPD pattern of tiagabine hydrochloride Form K contains peaks at 5.7, 13.3, 16.6, 20.1, 20.6, 23.6, 24.5, and 24.9±0.2 degrees 2θ. A representative XRPD pattern of tiagabine hydrochloride Form K is presented in FIG. 29.

Tiagabine hydrochloride Form K converts to a mixture of tiagabine hydrochloride Forms Q and B during storage.

Preferably, the tiagabine hydrochloride Form K of the present invention has a purity of at least about 50% (w/w). More preferably, the tiagabine hydrochloride Form K has a purity of at least about 70% (w/w). More preferably, the tiagabine hydrochloride Form K has a purity of at least about 90% (w/w).

Tiagabine Hydrochloride Form L

Tiagabine hydrochloride Form L may be prepared by crystallizing tiagabine hydrochloride from nitromethane.

The XRPD pattern of tiagabine hydrochloride Form L contains peaks at 7.7, 12.5, 14.5, 17.1, 21.1, 21.8, 24.6, 25.1, 26.2, and 28.0±0.2 degrees 2θ. A representative XRPD pattern of tiagabine hydrochloride Form L is presented in FIG. 30.

Tiagabine hydrochloride Form L converts to a mixture of tiagabine hydrochloride Forms B and Q during storage.

Preferably, the tiagabine hydrochloride Form L of the present invention has a purity of at least about 50% (w/w). More preferably, the tiagabine hydrochloride Form L has a purity of at least about 70% (w/w). More preferably, the tiagabine hydrochloride Form L has a purity of at least about 90% (w/w).

Tiagabine Hydrochloride Form N

Tiagabine hydrochloride Form N may be prepared by crystallizing tiagabine hydrochloride from benzonitrile.

The XRPD pattern of tiagabine hydrochloride Form N contains peaks at 14.1, 14.5, 15.6, 17.1, 19.6, 22.6, 23.2, 23.8, 24.7, and 25.0±0.2 degrees 2θ. A representative XRPD pattern of tiagabine hydrochloride Form N is presented in FIG. 31.

Preferably, the tiagabine hydrochloride Form N of the present invention has a purity of at least about 50% (w/w). More preferably, the tiagabine hydrochloride Form N has a purity of at least about 70% (w/w). More preferably, the tiagabine hydrochloride Form N has a purity of at least about 90% (w/w).

Tiagabine Hydrochloride Form O

Tiagabine hydrochloride Form O may be prepared by heating tiagabine hydrochloride monohydrate.

The XRPD pattern of tiagabine hydrochloride Form O contains peaks at 12.6, 14.6, 16.4, 18.6, 18.9, 23.3, 24.3, and 25.9±0.2 degrees 2θ. A representative XRPD pattern of tiagabine hydrochloride Form O is presented in FIG. 32.

Preferably, the tiagabine hydrochloride Form O of the present invention has a purity of at least about 50% (w/w). More preferably, the tiagabine hydrochloride Form O has a purity of at least about 70% (w/w). More preferably, the tiagabine hydrochloride Form O has a purity of at least about 90% (w/w).

Tiagabine Hydrochloride Form R

Tiagabine hydrochloride Form R may be prepared by slurrying tiagabine hydrochloride monohydrate in nitromethane.

The XRPD pattern of tiagabine hydrochloride Form R contains peaks at 10.8, 13.0, 15.3, 16.7, 17.8, 22.2, 25.4, 26.9, 28.0, and 32.2±0.2 degrees 2θ. A representative XRPD pattern of tiagabine hydrochloride Form R is presented in FIG. 33.

Preferably, the tiagabine hydrochloride Form R of the present invention has a purity of at least about 50% (w/w). More preferably, the tiagabine hydrochloride Form R has a purity of at least about 70% (w/w). More preferably, the tiagabine hydrochloride Form R has a purity of at least about 90% (w/w).

Tiagabine Hydrochloride Form U

Tiagabine hydrochloride Form U may be prepared by slurrying tiagabine hydrochloride monohydrate in 1,2-dichloroethane.

The XRPD pattern of tiagabine hydrochloride Form U contains peaks at 12.6, 14.4, 16.4, 16.9, 21.2, 21.6, 22.9, 23.9, 26.6, and 27.6±0.2 degrees 2θ. A representative XRPD pattern of tiagabine hydrochloride Form U is presented in FIG. 34.

Preferably, the tiagabine hydrochloride Form U of the present invention has a purity of at least about 50% (w/w). More preferably, the tiagabine hydrochloride Form U has a purity of at least about 70% (w/w). More preferably, the tiagabine hydrochloride Form U has a purity of at least about 90% (w/w).

Tiagabine Hydrochloride Form V

Tiagabine hydrochloride Form V may be prepared by slurrying tiagabine hydrochloride monohydrate in 1,2-dimethoxyethane.

The XRPD pattern of tiagabine hydrochloride Form V contains peaks at 7.4, 11.6, 12.9, 15.8, 16.1, 18.5, 19.4, 21.2, 23.9, and 26.4±0.2 degrees 2θ. A representative XRPD pattern of tiagabine hydrochloride Form V is presented in FIG. 35.

Preferably, the tiagabine hydrochloride Form V of the present invention has a purity of at least about 50% (w/w). More preferably, the tiagabine hydrochloride Form V has a purity of at least about 70% (w/w). More preferably, the tiagabine hydrochloride Form V has a purity of at least about 90% (w/w).

Tiagabine Hydrochloride Form AC

Tiagabine hydrochloride Form AC may be prepared by crystallizing tiagabine hydrochloride from cyclohexanol.

The XRPD pattern of tiagabine hydrochloride Form AC contains peaks at 7.8, 8.5, 12.4, 14.7, 15.3, 15.8, 17.0, 18.2, 22.9, and 25.0±0.2 degrees 2θ. A representative XRPD pattern of tiagabine hydrochloride Form AC is presented in FIG. 36.

Preferably, the tiagabine hydrochloride Form AC of the present invention has a purity of at least about 50% (w/w). More preferably, the tiagabine hydrochloride Form AC has a purity of at least about 70% (w/w). More preferably, the tiagabine hydrochloride Form AC has a purity of at least about 90% (w/w).

Pharmaceutical Composition

The present invention provides a pharmaceutical composition comprising a pharmaceutically acceptable excipient and at least one tiagabine form chosen from tiagabine hydrochloride Forms G, K, L, N, O, R, U, V, and AC, tiagabine free base Forms A, B, C, D, E, F, G, and H, tiagabine free base amorphous, tiagabine camphorate Form A, tiagabine hydrobromide Form A, tiagabine dl-malate Form A, tiagabine d-malate Form A, tiagabine tartrate Form A, and tiagabine hydrochloride cocrystal with 2-furancarboxylic acid. Preferably, the tiagabine form is tiagabine hydrochloride Form G. Preferably, the tiagabine form is tiagabine hydrochloride Form K. Preferably, the tiagabine form is tiagabine hydrochloride Form L. Preferably, the tiagabine form is tiagabine hydrochloride Form N. Preferably, the tiagabine form is tiagabine hydrochloride Form O. Preferably, the tiagabine form is tiagabine hydrochloride Form R. Preferably, the tiagabine form is tiagabine hydrochloride Form U. Preferably, the tiagabine form is tiagabine hydrochloride Form V. Preferably, the tiagabine form is tiagabine hydrochloride Form AC. Preferably, the tiagabine form is tiagabine free base Form A. Preferably, the tiagabine form is tiagabine free base Form B. Preferably, the tiagabine form is tiagabine free base Form C. Preferably, the tiagabine form is tiagabine free base Form D. Preferably, the tiagabine form is tiagabine free base Form E. Preferably, the tiagabine form is tiagabine free base Form F. Preferably, the tiagabine form is tiagabine free base Form G. Preferably, the tiagabine form is tiagabine free base Form H. Preferably, the tiagabine form is tiagabine camphorate Form A. Preferably, the tiagabine form is tiagabine hydrobromide Form A. Preferably, the tiagabine form is tiagabine dl-malate Form A. Preferably, the tiagabine form is tiagabine d-malate Form A. Preferably, the tiagabine form is tiagabine tartrate Form A. Preferably, the tiagabine form is tiagabine free base amorphous form. Preferably, the tiagabine form is tiagabine hydrochloride cocrystal with 2-furancarboxylic acid.

Preferably, the pharmaceutical composition comprises a pharmaceutically acceptable excipient and at least one tiagabine form chosen from tiagabine free base Forms A, B, C, D, E, F, G, and H and tiagabine free base amorphous.

Further, there is provided a process for preparing such a pharmaceutical composition, comprising the step of mixing at least one tiagabine form chosen from tiagabine hydrochloride Forms G, K, L, N, O, R, U, V, and AC, tiagabine free base Forms A, B, C, D, E, F, G, and H, tiagabine free base amorphous, tiagabine camphorate Form A, tiagabine hydrobromide Form A, tiagabine dl-malate Form A, tiagabine d-malate Form A, tiagabine tartrate Form A, and tiagabine hydrochloride cocrystal with 2-furancarboxylic acid with a pharmaceutically acceptable excipient. Preferably, the tiagabine form is tiagabine hydrochloride Form G. Preferably, the tiagabine form is tiagabine hydrochloride Form K. Preferably, the tiagabine form is tiagabine hydrochloride Form L. Preferably, the tiagabine form is tiagabine hydrochloride Form N. Preferably, the tiagabine form is tiagabine hydrochloride Form O. Preferably, the tiagabine form is tiagabine hydrochloride Form R. Preferably, the tiagabine form is tiagabine hydrochloride Form U. Preferably, the tiagabine form is tiagabine hydrochloride Form V. Preferably, the tiagabine form is tiagabine hydrochloride Form AC. Preferably, the tiagabine form is tiagabine free base Form A. Preferably, the tiagabine form is tiagabine free base Form B. Preferably, the tiagabine form is tiagabine free base Form C. Preferably, the tiagabine form is tiagabine free base Form D. Preferably, the tiagabine form is tiagabine free base Form E. Preferably, the tiagabine form is tiagabine free base Form F. Preferably, the tiagabine form is tiagabine free base Form G. Preferably, the tiagabine form is tiagabine free base Form H. Preferably, the tiagabine form is tiagabine camphorate Form A. Preferably, the tiagabine form is tiagabine hydrobromide Form A. Preferably, the tiagabine form is tiagabine dl-malate Form A. Preferably, the tiagabine form is tiagabine d-malate Form A. Preferably, the tiagabine form is tiagabine tartrate Form A. Preferably, the tiagabine form is tiagabine free base amorphous form. Preferably, the tiagabine form is tiagabine hydrochloride cocrystal with 2-furancarboxylic acid.

Preferably, the process comprises the step of mixing at least one tiagabine form chosen from tiagabine free base Forms A, B, C, D, E, F, G, and H and tiagabine free base amorphous with a pharmaceutically acceptable excipient.

The present crystalline and amorphous forms of tiagabine free base and tiagabine salts may, for example, conveniently be formulated for topical, oral, buccal, sublingual, parenteral, local or rectal administration. Preferably, the pharmaceutical composition is a dry oral dosage form. Preferably, the pharmaceutical composition is an oral dosage form chosen from tablet, pill, capsule, caplet, powder, granule, and gel. Dry dosage forms may include pharmaceutically acceptable additives, such as excipients, carriers, diluents, stabilizers, plasticizers, binders, glidants, disintegrants, bulking agents, lubricants, plasticizers, colorants, film formers, flavoring agents, preservatives, dosing vehicles, and any combination of any of the foregoing.

Diluents increase the bulk of a solid pharmaceutical composition and may make a pharmaceutical dosage form containing the composition easier for the patient and caregiver to handle. Diluents for solid compositions include, but are not limited to, microcrystalline cellulose (e.g. AVICEL®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Binders for solid pharmaceutical compositions include, but are not limited to, acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. KLUCEL®), hydroxypropyl methyl cellulose (e.g. METHOCEL®), liquid glucose, magnesium aluminum silicate, maltodextrin, methylcellulose, polymethacrylates, povidone (e.g. KOLLIDON®, PLASDONE®), pregelatinized starch, sodium alginate and starch.

The dissolution rate of a compacted solid pharmaceutical composition in the patient's stomach may be increased by the addition of a disintegrant to the composition. Disintegrants include, but are not limited to, alginic acid, carboxymethylcellulose calcium, carboxymethylcellulose sodium (e.g. AC-DI-SOL®, PRIMELLOSE®), colloidal silicon dioxide, croscarmellose sodium, crospovidone (e.g. KOLLIDON®, POLYPLASDONE®), guar gum, magnesium aluminum silicate, methyl cellulose, microcrystalline cellulose, powdered cellulose, pregelatinized starch, sodium alginate, sodium starch glycolate (e.g. EXPLOTAB®) and starch.

Glidants can be added to improve the flow properties of non-compacted solid compositions and improve the accuracy of dosing. Excipients that may function as glidants include, but are not limited to, colloidal silicon dioxide, magnesium trisilicate, powdered cellulose, starch, talc and tribasic calcium phosphate.

When a dosage form such as a tablet is made by compaction of a powdered composition, the composition is subjected to pressure from a punch and die. Some excipients and active ingredients have a tendency to adhere to the surfaces of the punch and die, which can cause the product to have pitting and other surface irregularities. A lubricant can be added to the composition to reduce adhesion and ease release of the product from the die. Lubricants include, but are not limited to, magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid ethyl maltol, and tartaric acid.

Compositions may also be colored using any pharmaceutically acceptable colorant to improve their appearance and/or facilitate patient identification of the product and unit dosage level.

Selection of excipients and the amounts to use may be readily determined by formulation scientists based upon experience and consideration of standard procedures and reference works in the field. The solid compositions of the present invention include powders, granulates, aggregates and compacted compositions. The preferred route of the present invention is oral. The dosages may be conveniently presented in unit dosage form and prepared by any of the methods well-known in the pharmaceutical arts. Dosage forms include solid dosage forms like tablets, pills, powders, caplets, granules, capsules, sachets, troches and lozenges. An especially preferred dosage form of the present invention is a tablet.

Ointments, creams and gels, may, for example, be formulated with an aqueous or oily base with the addition of a suitable thickening agent, gelling agent, and/or solvent. Such bases may thus, for example, include water and/or an oil such as liquid paraffin or a vegetable oil such as arachis oil or castor oil, or a solvent such as polyethylene glycol. Thickening agents and gelling agents that may be used according to the nature of the base include, but are not limited to, soft paraffin, aluminum stearate, cetostearyl alcohol, polyethylene glycols, woolfat, beeswax, carboxypolymethylene and cellulose derivatives, and/or glyceryl monostearate and/or non-ionic emulsifying agents.

Lotions may be formulated with an aqueous or oily base and will in general also contain one or more emulsifying agents, stabilizing agents, dispersing agents, suspending agents or thickening agents. Powders for external application may be formed with the aid of any suitable powder base, for example, talc, lactose or starch. Drops may be formulated with an aqueous or non-aqueous base also comprising one or more dispersing agents, solubilizing agents, suspending agents or preservatives.

If appropriate, the formulations of the invention may be buffered by the addition of suitable buffering agents.

Preferably, the pharmaceutical composition of the present invention is a unit dose composition. Preferably, the pharmaceutical composition of the present invention contains about 1 to 200 mg of the tiagabine form. More preferably, the pharmaceutical composition contains about 2 to 100 mg of the tiagabine form. More preferably, the pharmaceutical composition contains about 2 to 50 mg of the tiagabine form. More preferably, the pharmaceutical composition contains about 2 mg, 4 mg, 8 mg, 10 mg, 12 mg, 16 mg, 20 mg, 25 mg, or 30 mg of the tiagabine form. More preferably, the pharmaceutical composition contains about 2 mg, 4 mg, 12 mg, or 16 mg of the tiagabine form.

Method of Treatment

The present invention provides a method of treating a disease related to GABA uptake in a mammal, comprising the step of administering to the mammal a therapeutically effective amount of at least one tiagabine form chosen from tiagabine hydrochloride Forms G, K, L, N, O, R, U, V, and AC, tiagabine free base Forms A, B, C, D, E, F, G, and H, tiagabine free base amorphous, tiagabine camphorate Form A, tiagabine hydrobromide Form A, tiagabine dl-malate Form A, tiagabine d-malate Form A, tiagabine tartrate Form A, and tiagabine hydrochloride cocrystal with 2-furancarboxylic acid. Preferably, the tiagabine form is tiagabine hydrochloride Form G. Preferably, the tiagabine form is tiagabine hydrochloride Form K. Preferably, the tiagabine form is tiagabine hydrochloride Form L. Preferably, the tiagabine form is tiagabine hydrochloride Form N. Preferably, the tiagabine form is tiagabine hydrochloride Form O. Preferably, the tiagabine form is tiagabine hydrochloride Form R. Preferably, the tiagabine form is tiagabine hydrochloride Form U. Preferably, the tiagabine form is tiagabine hydrochloride Form V. Preferably, the tiagabine form is tiagabine hydrochloride Form AC. Preferably, the tiagabine form is tiagabine free base Form A. Preferably, the tiagabine form is tiagabine free base Form B. Preferably, the tiagabine form is tiagabine free base Form C. Preferably, the tiagabine form is tiagabine free base Form D. Preferably, the tiagabine form is tiagabine free base Form E. Preferably, the tiagabine form is tiagabine free base Form F. Preferably, the tiagabine form is tiagabine free base Form G. Preferably, the tiagabine form is tiagabine free base Form H. Preferably, the tiagabine form is tiagabine camphorate Form A. Preferably, the tiagabine form is tiagabine hydrobromide Form A. Preferably, the tiagabine form is tiagabine dl-malate Form A. Preferably, the tiagabine form is tiagabine d-malate Form A. Preferably, the tiagabine form is tiagabine tartrate Form A. Preferably, the tiagabine form is tiagabine free base amorphous form. Preferably, the tiagabine form is tiagabine hydrochloride cocrystal with 2-furancarboxylic acid.

Preferably, the method comprises the step of administering to the mammal a therapeutically effective amount of at least one tiagabine form chosen from tiagabine free base Forms A, B, C, D, E, F, G, and H and tiagabine free base amorphous.

Preferably, the disease related to GABA uptake is at least one disease chosen from epilepsy and partial seizures. Preferably, the disease related to GABA uptake is epilepsy. Preferably, the disease related to GABA uptake is partial seizures.

Preferably, the therapeutically effective amount is 1 to 500 mg per day. More preferably, the therapeutically effective amount is 1 to 100 mg per day. More preferably, the therapeutically effective amount is 4 to 60 mg per day.

Methodology and Protocols

X-Ray Powder Diffraction

X-ray powder diffraction (XRPD) analyses were performed using the following instruments & methods:

A. Shimadzu XRD-6000 X-ray powder diffractometer using Cu Kα radiation. The instrument was equipped with a long fine focus X-ray tube. The tube voltage and amperage were set to 40 kV and 40 mA, respectively. The divergence and scattering slits were set at 1° and the receiving slit was set at 0.15 mm. Diffracted radiation was detected by a Nal scintillation detector. A θ-2θ continuous scan at 3°/min (0.4 sec/0.02° step) from 2.5 to 40°2θ was used. A silicon standard was analyzed to check the instrument alignment. Data were collected and analyzed using XRD-6000 v. 4.1. Samples were prepared for analysis by placing them in a sample holder.

B. Inel XRG-3000 diffractometer, equipped with a CPS (Curved Position Sensitive) detector with a 2θ range of 120°. Real time data were collected using Cu—Kα radiation starting at approximately 4°2θ at a resolution of 0.03°2θ. The tube voltage and amperage were set to 40 kV and 30 mA, respectively. The monochromator slit was set at 5 mm by 80 μm or 160 μm. The pattern is displayed from 2.5-40°2θ. An aluminum sample holder was used or samples were prepared for analysis by packing them into thin-walled glass capillaries. Each capillary was mounted onto a goniometer head that is motorized to permit spinning of the capillary during data acquisition. The acquisition time was between 5 to 10 min. Instrument calibration was performed using a silicon reference standard.

C. Shimadzu XRD-6000 X-ray powder diffractometer equipped with an Anton Paar HTK 1200 high temperature stage (Variable-temperature XRPD (VT-XRPD)). The sample was packed in a ceramic holder and analyzed form 2.5 to 40°2θ at 3°/min (0.4 sec/0.02° step). The heating rate was 10° C./min. A silicon standard was analyzed to check the instrument alignment. Temperature calibration was performed using vanillin and sulfapyridine USP melting point standards. Data were collected and analyzed using XPD-6000 v.4.1.

D. Bruker D-8 Discover diffractometer and Bruker's General Area Diffraction Detection System (GADDS, v. 4.1.20). An incident beam of Cu—Kα radiation was produced using a fine-focus tube (40 kV, 40 mA), a Gobel mirror, and a 0.5 mm double-pinhole collimator. The samples were positioned for analysis by securing the well plate to a translation stage and moving each sample to intersect the incident beam. Alternatively, the sample was packed between 3-micron thick films to form a portable disc-shaped specimen, and the specimen was loaded in a holder secured to a translation stage. The samples were analyzed using a transmission geometry. The incident beam was scanned and rastered over the sample during the analysis to optimize orientation statistics. A beam-stop was used to minimize air scatter from the incident beam at low angles. Diffraction patterns were collected using a Hi-Star area detector located 15 cm from the sample and processed using GADDS. The intensity in the GADDS image of the diffraction pattern was integrated using a step size of 0.04°2θ. The integrated patterns display diffraction intensity as a function of 2θ. Prior to the analysis a silicon standard was analyzed to verify the Si 111 peak position.

E. Peak Picking Methods. Any XRPD files generated from Inel or Bruker XRPD instruments were converted to Shimadzu raw file using File Monkey version 3.0.4. The Shimadzu raw file was processed by the Shimadzu XRD-6000 version 4.1 software to automatically find peak positions. The “peak position” means the maximum intensity of a peaked intensity profile. The following processes were used with the Shimadzu XRD-6000 “Basic Process” version 2.6 algorithm:
Smoothing was done on all patterns.
The background was subtracted to find the net, relative intensity of the peaks.
A peak from Cu K alpha2 (1.5444 Å) wavelength was subtracted from the peak generated by Cu K alpha1 (1.5406 Å) peak at 50% intensity for all patterns.
   Differential Scanning Calorimetry

Differential scanning calorimetry (DSC) was performed using a TA Instruments differential scanning calorimeter 2920. The sample was placed into an aluminum DSC pan, and the weight accurately recorded. The pan was covered with a lid and then crimped. The sample cell was equilibrated at ambient temperature and heated under a nitrogen purge at a rate of 10° C./min, up to a final temperature of 350° C. or 375° C. Indium metal was used as the calibration standard. Reported temperatures are at the transition maxima.

Thermogravimetry

Standard thermogravimetry (TG) analyses were performed using a TA Instruments 2950 thermogravimetric analyzer. Each sample was placed in an aluminum sample pan and inserted into the TG furnace. The furnace was heated under nitrogen at a rate of 10° C./min, up to a final temperature of 350° C. Nickel and Alumel™ were used as the calibration standards.

Proton Solution Nuclear Magnetic Resonance

Solution ¹H NMR spectra were acquired at ambient temperature on a GE 300 MHz NMR spectrometer operating at 300.156250 MHz. The samples were prepared by dissolving approximately 4 mg of sample in 1.5 mL of NMR-grade DMSO-d₆. Spectra were acquired with a 1H pulse, a 1.36 second acquisition time, a 2.00 second delay between scans, a spectral width of 3012.0 Hz with 16384 data points, and 16 co-added scans. Each free induction decay (FID) was processed with NutsPro-2D Professional Version using a Fourier number equal to twice the number of acquired points. Peak tables were generated by the NutsPro software peak picking algorithm. Spectra were referenced to the residual ¹H peaks of the solvent (2.49 ppm vs. TMS at 0.0 ppm) as a secondary standard.

Alternatively, a solution ¹H nuclear magnetic resonance (NMR) spectrum was acquired at ambient temperature with a Varian ^{UNITY}INOVA-400 spectrometer at a ¹H Larmor frequency of 399.80 MHz. The sample was dissolved in DMSO-d₆ or CDCl₃. The free induction decay (FID) was processed using the Varian VNMR 6.1B software with various points and an exponential line broadening factor of 0.20 Hz to improve the signal-to-noise ratio. The spectrum was referenced to internal tetramethylsilane (TMS).

Moisture Sorption/Desorption

Moisture sorption/desorption data were collected on a VTI SGA-100 moisture balance system. For sorption isotherms, a sorption range of 5 to 95% relative humidity (RH) and a desorption range of 95 to 5% RH in 10% RH increments were used for analysis. The samples were not dried prior to analysis. Equilibrium criteria used for analysis were less than 0.0100% weight change in 5 minutes with a maximum equilibration time of 3 hours if the weight criterion was not met. Data were not corrected for the initial moisture content of the samples.

Hot Stage Microscopy

Hot stage microscopy was performed using a Linkam hotstage mounted on a Leica DM LP microscope. Samples were observed using a 20×0.4 NA objective a lambda plate with crossed polarizers. Another coverslip was then placed over the sample. Each sample was visually observed as the stage was heated. Images were captured using a SPOT Insight™ color digital camera with SPOT Software v. 3.5.8. The hotstage was calibrated using USP melting point standards.

### EXAMPLES

### Preparation 1. Tiagabine Free Base

Method A

(1) Tiagabine hydrochloride monohydrate (4.12 g) was dissolved in a solution of NaHCO₃ (0.84 g) in H₂O (40 mL) to give a clear yellow solution. The solution was extracted with dichloromethane (40 mL×2) and the organic phases combined and dried over MgSO₄. The MgSO₄ was removed by filtration and the filtrate was concentrated by rotary evaporation. The resulting residue was dissolved in ethanol.
(2) Tiagabine hydrochloride monohydrate (4.12 g) was suspended in H₂O (20 mL). A solution of NaHCO₃ (0.88 g) in H₂O (20 mL) was added, resulting in a clear solution. The solution was extracted with CH₂Cl₂ (30 mL×2). The organic phases were combined and dried over MgSO₄. The MgSO₄ was removed by filtration and the filtrate was concentrated to a residue.
(3) Tiagabine hydrochloride monohydrate (125.1 g, 0.304 mol) was suspended in H₂O (200 mL). A suspension of NaHCO₃ (28.0 g, 0.333 mol) in H₂O (300 mL) was added over a period of two (2) hours. The mixture was stirred for one (1) hour at ambient temperature, resulting in a clear solution. The solution was extracted with dichloromethane (1000 mL×1; 500 mL×1) and the organic phases combined. After drying over MgSO₄, the solution was filtered and the filtrate concentrated to a foam.
   Method B
(1) Tiagabine hydrochloride monohydrate (4.12 g, 0.01 mol) was suspended in dichloromethane (100 mL). A solution of NaOH (0.38 g, 0.0095 mol) in H₂O (5 mL) was added and the mixture was stirred for one (1) hour at ambient temperature to give an almost clear solution. NaHCO₃ (0.17 g, 0.002 mol) was added and the mixture was stirred for another one (1) hour at ambient temperature. The organic phase was separated and dried over MgSO₄. The MgSO₄ was removed by filtration and the filtrate concentrated to an oil. The oil was dissolved in ethanol (20 mL), seeded with tiagabine free base Form A, and refrigerated. The resulting precipitate was collected by filtration and dried under vacuum at ambient temperature for about four (4) hours. XRPD analysis of the sample indicated a mixture of tiagabine free base Forms A and B.
(2) Tiagabine hydrochloride monohydrate (113.5 g, 0.275 mol)) was suspended in dichloromethane (1000 mL). A solution of NaOH (10.46 g, 0.262 mol) in H₂O (150 mL) was added over a period of 30 minutes. The mixture was stirred for two (2) hours at ambient temperature. NaHCO₃ (4.63 g, 0.055 mol) was added and the mixture was stirred for another two (2) hours at ambient temperature. The organic phase was separated and the aqueous layer extracted with an additional 200 mL of dichloromethane. The organic phases were combined and dried over MgSO₄. The MgSO₄ was removed by filtration and the filtrate concentrated to a foam.
(3) Tiagabine hydrochloride monohydrate (103.0 g, 0.25 mol)) was suspended in CH₂Cl₂ (1000 mL). A solution of NaOH (9.5 g, 0.238 mol) in H₂O (150 mL) was added over a period of 30 minutes. The mixture was stirred for one (1) hour at ambient temperature. NaHCO₃ (4.2 g, 0.05 mol) was added and the mixture stirred for another two (2) hours at ambient temperature. The organic phase was separated and the aqueous layer extracted with an additional 200 mL of dichloromethane. The organic phases were combined and dried over MgSO₄. After filtering off the MgSO₄, the filtrate was concentrated to an off-white foam.
   Method C

A 0.1M phosphate buffer was generated by dissolving 1.29 g of sodium phosphate monobasic and 1.39 g of sodium phosphate dibasic (anhydrous) in 120 mL of water. The solution pH was ˜6 using colorPhast strips. Tiagabine hydrochloride monohydrate (2.15 g) and NaOH (0.20 g) were dissolved in 90 mL of the buffer. The resulting solution was extracted with of dichloromethane (3×150 mL). The organic layer was separated, dried with anhydrous magnesium sulfate, filtered and evaporated to dryness to give a light yellow solid (crude yield=1.74 g).

Method D

A 0.1M phosphate buffer was generated by dissolving 2.58 g of sodium phosphate monobasic and 2.78 g of sodium phosphate dibasic (anhydrous) in 240 ml of water. The pH was found to be ˜6 using colorPhast strips. Tiagabine hydrochloride monohydrate (4.31 g) and 0.40 g of NaOH were dissolved in 180 mL of the buffer. Sonication was used to assist in the dissolution of the solid. The flask was shielded from exposure to light. The resulting solution was extracted with dichloromethane (3×300 mL). The organic layer was separated, dried with anhydrous magnesium sulfate, filtered and evaporated to dryness to give a light yellow solid (crude yield=3.28 g). This product was dissolved in a minimal amount of hot ethanol using sonication to assist in the dissolution. The solution was filtered through a 0.2 μm syringe filter into a clean vial. The solution was allowed to stand at 3° C. for 24 hours. The resulting solid was collected by filtration and allowed to dry at room temperature. The solid was stored in a vacuum desiccator (yield=2.55 g).

A dichloromethane solution was prepared by dissolving 182 mg of the resulting tiagabine free base in 5 mL of dichloromethane. The solution was filtered through a 20 μm filter prior to use.

### Preparation 2. Well Plate Experiments

The following general procedure was used for well plate experiments described herein: 50 μL of the tiagabine free base solution in dichloromethane obtained in Preparation 1, Method D is delivered to the well in a well plate. The solvent is evaporated under high vacuum for 4 hours, producing a clear glass. To the well is added a solvent or mixture of solvents (50 μL). The plate is then sealed and stored at 3° C. for 24 hours. Optionally, one of more of the following additional steps may be performed to further promote crystal formation:

(a) a precipitating solvent (30 μL) may be added to the well;

(b) the sample may be stored at −17° C. for five (5) days; and/or

(c) the seal may be replaced with a foil cover having a pin hole, and the solvent allowed to slowly evaporate at room temperature.

### Preparation 3. Crystallization of Tiagabine Free Base

(1) The tiagabine free base samples obtained in Preparation 1 Method A(3), Method B(2), and Method B(3) were combined and dissolved into ethanol (400 mL). The resulting brown solution was seeded with tiagabine free base Form A obtained in Example 1, Method 1 and refrigerated. A white precipitate formed. Ethanol (200 mL) was added and mixture was slurried under nitrogen for four (4) hours. The solids were collected by filtration and rinsed with ethanol. The solids were dried under vacuum for approximately 2 days (yield=220.1 g).
The filtrate was concentrated to a brown residue. The residue was dissolved in ethanol (200 mL), seeded with tiagabine free base Form A obtained by Example 1, Method 2 and placed in a refrigerator. A white precipitate formed. Ethanol (200 mL) was added and the solids were collected by filtration and rinsed with ethanol. The solids were dried under vacuum at ambient temperature overnight (yield=38.6 g).
(2) The filtrate obtained in Preparation 3(1) was combined with the filtrate obtained in Example 1, Method 3 (below), and the combined filtrates were concentrated on a rotary evaporator to give a sticky residue. The residue was dissolved in ethanol (200 mL) and the resulting solution was seeded with tiagabine free base Form A obtained in Example 1, Method 3. Isopropyl ether (200 mL) was added and the solution was refrigerated overnight. The resulting white precipitate was collected by filtration and rinsed with ethanol (50 mL). The white solids were air-dried. (Yield=12 g).

### Preparation 4. Tiagabine Hydrochloride Amorphous

Preparation Method 1

0.1 g of tiagabine HCl was placed in a vial. The sample was heated at 204° C. in an oil bath under vacuum for about 5 minutes. The sample was completely melted. The sample was then crash-cooled by immersing in an ice bath. The glassy solids were ground in a mortar into small plates before analysis. The obtained product was amorphous, composed of small plates, and without birefringence.

Preparation Method 2

0.1 g of tiagabine HCl was placed in a vial. The sample was placed under a gentle nitrogen stream and then heated at 200° C. in an oil bath for one minute. The sample was completely melted. The sample was heated in the bath for an additional 3 minutes before it was immersed in a dry ice/isopropanol bath. The obtained product was amorphous, brown/dark yellow in color, glassy, and without birefringence.

Preparation Method 3

0.2 g of tiagabine HCl was dissolved in 20 mL of water to give a clear solution. The solution was filtered through a 0.2 μm filter. The filtrate was frozen in a dry ice/acetone bath, and then dried in a freeze dryer under high vacuum.

Preparation Method 4

Tiagabine HCl form B (32 mg) was placed in a grinding jar with a 5 mm stainless steel ball. The sample was milled for 10 minute intervals (3×10 minutes=30 minutes) at 30 Hz using a Retsch MM200 mixer mill. Solids were scraped from the sides of the vial after each interval. Sample was collected in a vial.

### Example 1

### Preparation and Characterization of Tiagabine Free Base Form A

Method 1

The crude tiagabine free base obtained in Preparation 1, Method C (1.74 g) was dissolved in hot ethanol with stirring. The solution was filtered through a 0.2 μm syringe filter into a clean vial. The solution was allowed to stand at 3° C. After 24 hours the resulting sold was collected by filtration and allowed to dry at room temperature (yield=1.04 g).

Method 2

The tiagabine free base samples obtained in Preparation 1 Method A(1) and Method A(2) were combined and dissolved in ethanol (ca. 20 mL). The solution was seeded with tiagabine free base Form A obtained in Example 1, Method 1 and refrigerated for about 4 hours. A white precipitate formed. The solids were collected by filtration, rinsed with ethanol (20 mL), and dried under vacuum at ambient temperature for about 3 hours.

Method 3

The dried solids obtained in Preparation 3(1) were combined (259 g) and slurried at room temperature for three (3) days in hexane (1,000 mL). Isopropyl ether (200 mL) and ethanol (30 mL) were added, and the resulting mixture was agitated by sonication or stirring for an additional two (2) days. The resulting off-white solids were collected by filtration and air dried.

XRPD

A representative XRPD pattern of tiagabine free base Form A is presented in FIG. 1. Representative peaks are listed in the following Table 4.

TABLE 4
Tiagabine Free Base Form A XRPD Peaks

| Peak No.^{a} | Position (°2θ)^{a} | d-spacing | I/Iₒ^{b} |
|---|---|---|---|
| 1 | 5.2 | 17.0 | 32 |
| 2 | 6.5 | 13.6 | 38 |
| 3 | 8.1 | 10.9 | 34 |
| 4 | 10.3 | 8.6 | 25 |
| 5 | 10.7 | 8.3 | 36 |
| 6 | 11.8 | 7.5 | 16 |
| 7 | 12.2 | 7.3 | 10 |
| 8 | 12.6 | 7.0 | 68 |
| 9 | 13.1 | 6.8 | 16 |
| 10 | 13.9 | 6.4 | 7 |
| 11 | 14.3 | 6.2 | 10 |
| 12 | 15.3 | 5.8 | 73 |
| 13 | 16.0 | 5.5 | 64 |
| 14 | 16.2 | 5.5 | 90 |
| 15 | 16.8 | 5.3 | 39 |
| 16 | 17.4 | 5.1 | 100 |
| 17 | 19.0 | 4.7 | 94 |
| 18 | 19.5 | 4.5 | 51 |
| 19 | 21.4 | 4.2 | 58 |
| 20 | 21.7 | 4.1 | 18 |
| 21 | 22.1 | 4.0 | 57 |
| 22 | 22.4 | 4.0 | 35 |
| 23 | 22.9 | 3.9 | 69 |
| 24 | 23.5 | 3.8 | 37 |
| 25 | 23.9 | 3.7 | 12 |
| 26 | 24.4 | 3.7 | 45 |
| 27 | 24.9 | 3.6 | 29 |
| 28 | 25.3 | 3.5 | 38 |
| 29 | 25.8 | 3.5 | 47 |
| 30 | 26.4 | 3.4 | 30 |
| 31 | 27.2 | 3.3 | 14 |
| 32 | 27.8 | 3.2 | 16 |
| 33 | 28.2 | 3.1 | 16 |
| 34 | 28.6 | 3.1 | 27 |
| 35 | 29.1 | 3.1 | 19 |
| 36 | 30.3 | 2.9 | 19 |
| 37 | 31.0 | 2.9 | 15 |

^{a}Bold: Unique set of XRPD Peaks for tiagabine free base Form A.
^{b}Intensity of peak/Intensity of most intense peak

DSC

DSC analysis indicated a major endotherm at 56° C. A representative DSC curve of tiagabine free base Form A is presented in FIG. 2.

TGA

TGA analysis indicated a 2.9% weight loss to 82° C., and a 4.7% weight loss to 167° C. A representative TGA curve of tiagabine free base Form A is presented in FIG. 2.

Moisture Sorption/Desorption

Moisture sorption/desorption analysis indicated a 1.0% weight loss upon equilibration at 5% relative humidity (RH), a 23.5% weight gain from 5% to 95% RH, and a 18.7% weight loss from 95% to 5% RH. XRPD analysis of the sample after moisture sorption/desorption indicated tiagabine free base amorphous.

¹H NMR

¹H NMR analysis indicated that the tiagabine free base Form A contained 0.22 moles of ethanol per mole of tiagabine free base.

Hot Stage Microscopy

Hot stage microscopy indicated a melt onset of 55.1° C. for tiagabine free base Form A.

### Example 2

### Preparation and Characterization of Tiagabine Free Base Form B

Method 1

Tiagabine free base Form A (prepared in Example 1, Method 3) (approximately 0.2 g) was dried for three (3) days under vacuum at room temperature.

Method 2

A well plate experiment was performed as in Preparation 2 using a mixture of tetrahydrofuran and isopropanol (2:1, v/v) as the solvent. No precipitating solvent was added. The seal was replaced with a foil cover containing one pin hole per well and the solvent was allowed to evaporate at room temperature.

Method 3

A well plate experiment was performed as in Preparation 2 using ethanol as the solvent. No precipitating solvent was added. The sample was then stored at −17° C. for five (5) days, and then the solvent was allowed to evaporate at room temperature.

XRPD

A representative XRPD pattern of tiagabine free base Form B is presented in FIG. 3. Representative peaks are listed in the following Table 5.

TABLE 5
Tiagabine Free Base Form B XRPD Peaks

| Peak No.^{a} | Position (°2θ) | d-spacing | I/Iₒ^{b} |
|---|---|---|---|
| 1 | 7.2 | 12.3 | 27 |
| 2 | 8.1 | 10.9 | 20 |
| 3 | 9.9 | 8.9 | 19 |
| 4 | 10.6 | 8.3 | 14 |
| 5 | 11.0 | 8.1 | 17 |
| 6 | 11.7 | 7.6 | 9 |
| 7 | 12.5 | 7.1 | 69 |
| 8 | 13.3 | 6.7 | 10 |
| 9 | 13.9 | 6.4 | 10 |
| 10 | 14.3 | 6.2 | 26 |
| 11 | 15.0 | 5.9 | 72 |
| 12 | 15.4 | 5.7 | 97 |
| 13 | 16.4 | 5.4 | 70 |
| 14 | 17.0 | 5.2 | 36 |
| 15 | 17.3 | 5.1 | 23 |
| 16 | 18.0 | 4.9 | 7 |
| 17 | 18.6 | 4.8 | 11 |
| 18 | 19.1 | 4.7 | 18 |
| 19 | 19.6 | 4.5 | 29 |
| 20 | 19.9 | 4.5 | 41 |
| 21 | 20.3 | 4.4 | 4 |
| 22 | 20.8 | 4.3 | 7 |
| 23 | 21.3 | 4.2 | 93 |
| 24 | 22.5 | 4.0 | 100 |
| 25 | 23.0 | 3.9 | 30 |
| 26 | 23.6 | 3.8 | 10 |
| 27 | 24.1 | 3.7 | 9 |
| 28 | 24.8 | 3.6 | 65 |
| 29 | 25.5 | 3.5 | 20 |
| 30 | 26.6 | 3.4 | 25 |
| 31 | 27.4 | 3.3 | 19 |
| 32 | 27.9 | 3.2 | 30 |
| 33 | 29.0 | 3.1 | 4 |
| 34 | 29.6 | 3.0 | 5 |
| 35 | 30.2 | 3.0 | 18 |
| 36 | 31.7 | 2.8 | 4 |

^{a}Bold: Unique set of XRPD Peaks for tiagabine free base Form B.
^{b}Intensity of peak/Intensity of most intense peak

DSC

DSC analysis indicated a major endotherm at 56° C. A representative DSC curve of tiagabine free base Form B is presented in FIG. 4.

TGA

TGA analysis indicated a 1.4% weight loss to 90° C., and a 2.5% weight loss to 175° C.

Hot Stage Microscopy

Hot stage microscopy indicated a melt onset of 55.2° C. for tiagabine free base Form B.

### Example 3

### Preparation and Characterization of Tiagabine Free Base Form C

Method 1

Tiagabine free base Form A from Example 1, Method 3 (0.1 g) was slurried in isopropanol (7 mL) for 3 days at room temperature. The liquid phase was removed by decantation and the solids were air-dried.

Method 2

The decanted solvent from Example 3 Method 1 was refrigerated. A few precipitates were observed prior to refrigeration. After three days the liquid phase was removed by decantation and the solids formed were dried under a nitrogen atmosphere for approximately 5 hours.

Method 3

A well plate experiment was performed as in Preparation 2 using acetonitrile as the solvent. No precipitating solvent was added.

Method 4

A well plate experiment was performed as in Preparation 2 using ethanol as the solvent. No precipitating solvent was added. The sample was then stored at −17° C. for five (5) days, and then the solvent was allowed to evaporate at room temperature.

Method 5

A well plate experiment was performed as in Preparation 2 using isopropanol as the solvent and cyclohexane as the precipitating solvent. The sample was then stored at −17° C. for five (5) days, and then the solvent was allowed to evaporate at room temperature.

Method 6

A well plate experiment was performed as in Preparation 2 using a mixture of tetrahydrofuran and isopropanol (2:1, v/v) as the solvent and acetonitrile as the precipitating solvent. The sample was then stored at −17° C. for five (5) days, and then the solvent was allowed to evaporate at room temperature.

XRPD

A representative XRPD pattern of tiagabine free base Form C is presented in FIG. 5. Representative peaks are listed in the following Table 6.

TABLE 6
Tiagabine Free Base Form C XRPD Peaks

| Peak No.^{a} | Position (°2θ) | d-spacing | I/Iₒ^{b} |
|---|---|---|---|
| 1 | 4.9 | 17.9 | 42 |
| 2 | 6.1 | 14.5 | 47 |
| 3 | 7.8 | 11.3 | 30 |
| 4 | 9.9 | 8.9 | 86 |
| 5 | 12.2 | 7.3 | 26 |
| 6 | 12.6 | 7.1 | 4 |
| 7 | 12.9 | 6.8 | 33 |
| 8 | 13.4 | 6.6 | 5 |
| 9 | 14.1 | 6.3 | 16 |
| 10 | 14.3 | 6.2 | 12 |
| 11 | 14.5 | 6.1 | 11 |
| 12 | 14.8 | 6.0 | 5 |
| 13 | 15.4 | 5.8 | 14 |
| 14 | 15.6 | 5.7 | 33 |
| 15 | 16.1 | 5.5 | 75 |
| 16 | 16.8 | 5.3 | 20 |
| 17 | 17.1 | 5.2 | 22 |
| 18 | 18.3 | 4.9 | 86 |
| 19 | 18.7 | 4.7 | 100 |
| 20 | 19.2 | 4.6 | 65 |
| 21 | 19.8 | 4.5 | 57 |
| 22 | 20.0 | 4.4 | 8 |
| 23 | 20.6 | 4.3 | 38 |
| 24 | 21.0 | 4.2 | 10 |
| 25 | 21.3 | 4.2 | 8 |
| 26 | 21.8 | 4.1 | 20 |
| 27 | 22.9 | 3.9 | 10 |
| 28 | 23.3 | 3.8 | 9 |
| 29 | 24.0 | 3.7 | 62 |
| 30 | 24.5 | 3.6 | 57 |
| 31 | 24.8 | 3.6 | 21 |
| 32 | 25.1 | 3.5 | 38 |
| 33 | 25.5 | 3.5 | 5 |
| 34 | 26.0 | 3.4 | 37 |
| 35 | 26.5 | 3.4 | 7 |
| 36 | 27.1 | 3.3 | 7 |
| 37 | 27.3 | 3.3 | 3 |
| 38 | 28.3 | 3.2 | 27 |
| 39 | 28.8 | 3.1 | 26 |
| 40 | 29.1 | 3.1 | 8 |
| 41 | 29.4 | 3.0 | 14 |
| 42 | 30.0 | 3.0 | 4 |
| 43 | 31.3 | 2.9 | 9 |

^{a}Bold: Unique set of XRPD Peaks for tiagabine free base Form C.
^{b}Intensity of peak/Intensity of most intense peak

DSC

DSC analysis indicated a major endotherm at 75° C. A representative DSC curve of tiagabine free base Form C is presented in FIG. 6.

TGA

TGA analysis indicated a 5.8% weight loss to 95° C., and a 8.9% weight loss to 165° C.

Hot Stage Microscopy

Hot stage microscopy indicated a melt onset of 56.9° C. for tiagabine free base Form C.

### Example 4

### Preparation and Characterization of Tiagabine Free Base Form D

Method 1

Tiagabine free base Form A (8 mg) was dissolved in a 1:1 (v/v) mixture of 2,2,2-trifluoroethanol and methyl ethyl ketone (1/1). The solvent was allowed to slowly evaporate. The resultant residue was dissolved in methyl ethyl ketone (0.4 mL) and the solution was refrigerated. After 2 days some crystals were observed in the solution. The solvent was then evaporated under a gentle stream of nitrogen to afford solids.

Method 2

Tiagabine free base Form A (78 mg) was dissolved in trifluoroethanol (1 mL). The resulting clear solution was filtered using a 0.2 μm filter and the solvent allowed to evaporate slowly. The resultant glassy residue was dissolved in trifluoroethanol (0.4 mL) and refrigerated for 2 days, after which time no solids were present. The sample was placed, uncapped, in a desiccator under a nitrogen purge for three days resulting in a gum-like residue. Isopropyl ether (0.5 mL) was added and the mixture slurried at room temperature for 3 days. The liquid phase was decanted and the residue was dried under a nitrogen atmosphere.

Method 3

Tiagabine free base Form A (147 mg) was dissolved in methyl ethyl ketone (0.5 mL). The clear solution was filtered through a 0.2 um filter. The filtrate was seeded with tiagabine free base Form E and refrigerated. No solids were present after two days. The sample was removed from the refrigerator and the solvent was allowed to evaporate under nitrogen at ambient temperature. The resultant tacky residue was treated with trifluoroethanol (0.2 mL) and refrigerated for 3 days. The sample was allowed to equilibrate to ambient temperature in a desiccator and isopropyl ether (1.5 mL) was added resulting in a cloudy solution. After refrigeration for one day, the solvent was decanted and the solids dried in a desiccator under nitrogen.

XRPD

A representative XRPD pattern of tiagabine free base Form D is presented in FIG. 7. Representative peaks are listed in the following Table 7.

TABLE 7
Tiagabine Free Base Form D XRPD Peaks

| Peak No.^{a} | Position (°2θ) | d-spacing | I/I₀^{b} |
|---|---|---|---|
| 1 | 5.7 | 15.5 | 4 |
| 2 | 6.1 | 14.4 | 22 |
| 3 | 7.7 | 11.5 | 5 |
| 4 | 7.9 | 11.2 | 15 |
| 5 | 10.0 | 8.9 | 74 |
| 6 | 12.2 | 7.2 | 40 |
| 7 | 12.8 | 6.9 | 9 |
| 8 | 14.7 | 6.0 | 30 |
| 9 | 15.0 | 5.9 | 18 |
| 10 | 15.4 | 5.8 | 11 |
| 11 | 15.8 | 5.6 | 100 |
| 12 | 16.0 | 5.5 | 43 |
| 13 | 16.7 | 5.3 | 17 |
| 14 | 16.9 | 5.2 | 66 |
| 15 | 18.2 | 4.9 | 70 |
| 16 | 18.6 | 4.8 | 80 |
| 17 | 19.2 | 4.6 | 34 |
| 18 | 19.5 | 4.6 | 90 |
| 19 | 19.8 | 4.5 | 96 |
| 20 | 20.2 | 4.4 | 9 |
| 21 | 20.6 | 4.3 | 9 |
| 22 | 20.7 | 4.3 | 18 |
| 23 | 20.9 | 4.2 | 15 |
| 24 | 21.4 | 4.2 | 7 |
| 25 | 21.8 | 4.1 | 29 |
| 26 | 22.9 | 3.9 | 10 |
| 27 | 23.3 | 3.8 | 8 |
| 28 | 23.7 | 3.8 | 15 |
| 29 | 24.1 | 3.7 | 57 |
| 30 | 24.7 | 3.6 | 28 |
| 31 | 25.1 | 3.6 | 49 |
| 32 | 25.8 | 3.5 | 9 |

^{a}Bold: Unique set of XRPD Peaks for tiagabine free base Form D.
^{b}Intensity of peak/Intensity of most intense peak

DSC

DSC analysis indicated a major endotherm at 100° C. A representative DSC curve of tiagabine free base Form D is presented in FIG. 8.

TGA

TGA analysis indicated a 10.3% weight loss to 113° C., and a 18.6% weight loss to 183° C.

Hot Stage Microscopy

Hot stage microscopy indicated a melt onset of 59.9° C. for tiagabine free base Form D.

### Example 5

### Preparation and Characterization of Tiagabine Free Base Form E

Method 1

A well plate experiment was performed as in Preparation 2 using a mixture of propionitrile and t-butyl alcohol (1/1) as the solvent. No precipitating solvent was added. The plate was kept at 3° C. for 24 hours, and then the seal was replaced with a foil cover with one pin hole per well. The plate was allowed to slowly evaporate at room temperature.

Method 2

A well plate experiment was performed as in Preparation 2 using acetonitrile as the solvent and the precipitating solvent. The plate was stored at 3° C. for 24 hours prior to adding precipitating solvent. The sample was then stored at −17° C. for five (5) days, and then the solvent was allowed to evaporate at room temperature.

Method 3

A well plate experiment was performed as in Preparation 2 using a mixture of 2,2,2-trifluoroethanol and methyl ethyl ketone (1/1, v/v) as the solvent, and with or without using isopropyl ether as a precipitating solvent. The sample without isopropyl ether was then stored at 3° C. for 24 hours, and then allowed to slowly evaporate at room temperature. The sample with isopropyl ether was then stored at −17° C. for five (5) days, and then allowed to evaporate at room temperature.

XRPD

A representative XRPD pattern of tiagabine free base Form E is presented in FIG. 9. Representative peaks are listed in the following Table 8.

TABLE 8
Tiagabine Free Base Form E XRPD Peaks

| Peak No.^{a} | Position (°2θ) | d-spacing | I/I₀^{b} |
|---|---|---|---|
| 1 | 4.4 | 20.0 | 12 |
| 2 | 4.7 | 19.0 | 18 |
| 3 | 5.6 | 15.7 | 13 |
| 4 | 5.9 | 15.0 | 15 |
| 5 | 7.4 | 11.9 | 12 |
| 6 | 9.5 | 9.4 | 50 |
| 7 | 11.6 | 7.6 | 14 |
| 8 | 12.5 | 7.1 | 10 |
| 9 | 13.1 | 6.7 | 30 |
| 10 | 14.3 | 6.2 | 28 |
| 11 | 14.9 | 5.9 | 35 |
| 12 | 15.4 | 5.7 | 40 |
| 13 | 16.1 | 5.5 | 97 |
| 14 | 16.9 | 5.3 | 21 |
| 15 | 18.0 | 4.9 | 73 |
| 16 | 18.3 | 4.8 | 100 |
| 17 | 18.7 | 4.7 | 86 |
| 18 | 20.2 | 4.4 | 34 |
| 19 | 20.4 | 4.4 | 36 |
| 20 | 20.8 | 4.3 | 26 |
| 21 | 22.5 | 4.0 | 27 |
| 22 | 23.4 | 3.8 | 18 |
| 23 | 24.4 | 3.7 | 34 |
| 24 | 25.0 | 3.6 | 40 |
| 25 | 25.2 | 3.5 | 29 |
| 26 | 25.6 | 3.5 | 13 |
| 27 | 26.3 | 3.4 | 27 |
| 28 | 28.3 | 3.2 | 24 |
| 29 | 28.9 | 3.1 | 11 |
| 30 | 31.0 | 2.9 | 24 |

^{a}Bold: Unique set of XRPD Peaks for tiagabine free base Form E.
^{b}Intensity of peak/Intensity of most intense peak

### Example 6

### Preparation and Characterization of Tiagabine Free Base Form F

Tiagabine free base Form A (120 mg) was dissolved in a 1:2 (v/v) mixture of methanol and 2-propyl ether (0.6 mL). The solution was placed in a refrigerator for 3 days and a white precipitate was formed. The liquid phase was removed by decantation. The solids were dried under nitrogen atmosphere.

XRPD

A representative XRPD pattern of tiagabine free base Form F is presented in FIG. 10. Representative peaks are listed in the following Table 9.

TABLE 9
Tiagabine Free Base Form F XRPD Peaks

| Peak No.^{a} | Position (°2θ) | d-spacing | I/I₀^{b} |
|---|---|---|---|
| 1 | 6.3 | 14.0 | 14 |
| 2 | 8.0 | 11.0 | 15 |
| 3 | 10.0 | 8.8 | 35 |
| 4 | 10.5 | 8.5 | 28 |
| 5 | 12.7 | 7.0 | 12 |
| 6 | 15.3 | 5.8 | 17 |
| 7 | 16.2 | 5.5 | 100 |
| 8 | 16.9 | 5.2 | 26 |
| 9 | 17.6 | 5.0 | 14 |
| 10 | 18.9 | 4.7 | 26 |
| 11 | 19.7 | 4.5 | 22 |
| 12 | 20.6 | 4.3 | 12 |
| 13 | 21.1 | 4.2 | 22 |
| 14 | 21.8 | 4.1 | 23 |
| 15 | 24.2 | 3.7 | 18 |
| 16 | 24.9 | 3.6 | 40 |
| 17 | 25.6 | 3.5 | 26 |
| 18 | 26.1 | 3.4 | 14 |
| 19 | 27.0 | 3.3 | 25 |
| 20 | 28.9 | 3.1 | 18 |
| 21 | 29.3 | 3.0 | 23 |

^{a}Bold: Unique set of XRPD Peaks for tiagabine free base Form F.
^{b}Intensity of peak/Intensity of most intense peak

DSC

DSC analysis indicated a major endotherm at 59° C. A representative DSC curve of tiagabine free base Form F is presented in FIG. 11.

TGA

TGA analysis indicated a 2.2% weight loss to 88° C., and a 4.7% weight loss to 157° C.

Hot Stage Microscopy

Hot stage microscopy indicated a complete melt at 63.5° C. for tiagabine free base Form F.

### Example 7

### Preparation and Characterization of Tiagabine Free Base Form G

Tiagabine free Form A (120 mg) was dissolved in 2-butanol (0.5 mL). The solution was placed in a refrigerator for 3 days and a white precipitate was formed. The solids were dried in a desiccator under nitrogen atmosphere and then under vacuum at ambient temperature for approximately 3 hours.

XRPD

A representative XRPD pattern of tiagabine free base Form G is presented in FIG. 12. Representative peaks are listed in the following Table 10.

TABLE 10
Tiagabine Free Base Form G XRPD Peaks

| Peak No.^{a} | Position (°2θ) | d-spacing | I/Iₒ^{b} |
|---|---|---|---|
| 1 | 6.0 | 14.8 | 13 |
| 2 | 7.4 | 11.9 | 10 |
| 3 | 7.6 | 11.6 | 13 |
| 4 | 9.7 | 9.1 | 44 |
| 5 | 11.8 | 7.5 | 16 |
| 6 | 12.8 | 6.9 | 6 |
| 7 | 13.0 | 6.8 | 15 |
| 8 | 14.7 | 6.0 | 5 |
| 9 | 15.4 | 5.8 | 47 |
| 10 | 16.1 | 5.5 | 68 |
| 11 | 16.6 | 5.4 | 17 |
| 12 | 16.9 | 5.3 | 19 |
| 13 | 17.7 | 5.0 | 10 |
| 14 | 18.1 | 4.9 | 83 |
| 15 | 18.5 | 4.8 | 100 |
| 16 | 19.0 | 4.7 | 91 |
| 17 | 19.3 | 4.6 | 34 |
| 18 | 20.4 | 4.4 | 32 |
| 19 | 20.7 | 4.3 | 15 |
| 20 | 21.1 | 4.2 | 12 |
| 21 | 21.6 | 4.1 | 17 |
| 22 | 22.6 | 3.9 | 23 |
| 23 | 23.4 | 3.8 | 12 |
| 24 | 23.7 | 3.8 | 16 |
| 25 | 24.2 | 3.7 | 34 |
| 26 | 24.7 | 3.6 | 71 |
| 27 | 25.5 | 3.5 | 32 |
| 28 | 26.1 | 3.4 | 18 |
| 29 | 26.8 | 3.3 | 8 |
| 30 | 27.0 | 3.3 | 10 |
| 31 | 27.3 | 3.3 | 3 |
| 32 | 27.8 | 3.2 | 6 |
| 33 | 28.2 | 3.2 | 33 |
| 34 | 28.8 | 3.1 | 19 |
| 35 | 29.4 | 3.0 | 12 |
| 36 | 30.3 | 3.0 | 4 |
| 37 | 31.1 | 2.9 | 18 |
| 38 | 31.3 | 2.9 | 14 |
| 39 | 38.0 | 2.4 | 9 |

^{a}Bold: Unique set of XRPD Peaks for tiagabine free base Form G.
^{b}Intensity of peak/Intensity of most intense peak

DSC

DSC analysis indicated a major endotherm at 57° C. A representative DSC curve of tiagabine free base Form G is presented in FIG. 13.

TGA

TGA analysis indicated a 6.2% weight loss to 87° C., and a 9.7% weight loss to 175° C.

Hot Stage Microscopy

Hot stage microscopy indicated a melt onset of 47.0° C. for tiagabine free base Form G.

### Example 8

### Preparation and Characterization of Tiagabine Free Base Form H

Tiagabine free base Form A (0.1 g) was dissolved in 1-propanol (0.5 mL). The solution was placed in a refrigerator for 3 days and a white precipitate was formed. The solids were dried under nitrogen in a desiccator, and then dried under vacuum at ambient temperature for approximately 3 hours.

XRPD

A representative XRPD pattern of tiagabine free base Form H is presented in FIG. 14. Representative peaks are listed in the following Table 11.

TABLE 11
Tiagabine Free Base Form H XRPD Peaks

| Peak No.^{a} | Position (°2θ) | d-spacing | I/Iₒ^{b} |
|---|---|---|---|
| 1 | 6.1 | 14.5 | 26 |
| 2 | 7.7 | 11.4 | 30 |
| 3 | 9.9 | 8.9 | 74 |
| 4 | 12.1 | 7.3 | 26 |
| 5 | 12.8 | 6.9 | 13 |
| 6 | 14.3 | 6.2 | 11 |
| 7 | 15.8 | 5.6 | 100 |
| 8 | 16.8 | 5.3 | 35 |
| 9 | 18.4 | 4.8 | 95 |
| 10 | 19.2 | 4.6 | 66 |
| 11 | 19.8 | 4.5 | 60 |
| 12 | 20.7 | 4.3 | 45 |
| 13 | 21.7 | 4.1 | 29 |
| 14 | 22.8 | 3.9 | 21 |
| 15 | 23.5 | 3.8 | 26 |
| 16 | 24.1 | 3.7 | 66 |
| 17 | 24.9 | 3.6 | 62 |
| 18 | 25.8 | 3.5 | 49 |
| 19 | 26.8 | 3.3 | 15 |
| 20 | 28.0 | 3.2 | 24 |
| 21 | 28.6 | 3.1 | 29 |
| 22 | 28.9 | 3.1 | 23 |
| 23 | 30.1 | 3.0 | 15 |
| 24 | 31.0 | 2.9 | 12 |
| 25 | 32.0 | 2.8 | 20 |
| 26 | 34.1 | 2.6 | 16 |
| 27 | 34.5 | 2.6 | 11 |
| 28 | 37.9 | 2.4 | 12 |
| 29 | 38.6 | 2.3 | 17 |

^{a}Bold: Unique set of XRPD Peaks for tiagabine free base Form H.
^{b}Intensity of peak/Intensity of most intense peak

### Example 9

### Preparation and Characterization of Tiagabine Free Base Amorphous

Method 1

Tiagabine free base Form A obtained in Example 1, Method 3 (0.284 g) was dried under vacuum at 43-46° C. for one day.

Method 2

A well plate experiment was performed as in Preparation 2 using 1,4-dioxane as the solvent. No precipitating solvent was added. After storing for 24 hours at 3° C., the seal was replaced with a foil cover with one pin hole per well. The solvent was allowed to slowly evaporate at room temperature to afford amorphous solid.

Method 3

A well plate experiment was performed as in Preparation 2 using isopropanol as the solvent. No precipitating solvent was added. After storing for 24 hours at 3° C., the seal was then replaced with a foil cover with one pin hole per well. The solvent was allowed to slowly evaporate at room temperature.

Method 4

A well plate experiment was performed as in Preparation 2 using 1,4-dioxane as the solvent and propyl ether as the precipitating solvent. Prior to addition of the precipitating solvent, the plate was sealed and stored at 3° C. for 24 hours. The sample was then stored at −17° C. for five (5) days, and then the solvent was allowed to evaporate at room temperature.

Method 5

Tiagabine free base Form A (156 mg) was dissolved in acetonitrile (3.5 mL) and dichloromethane (1 mL). The solution was filtered using a 0.2 μm filter and seeded with tiagabine free base Form E and refrigerated. White solids were collected after 2 days, collected by decantation and dried under nitrogen.

XRPD

A representative XRPD pattern of tiagabine free base amorphous is presented in FIG. 15.

### Example 10

### Preparation and Characterization of Tiagabine Camphorate Form A

Method 1

The tiagabine free base obtained in Preparation 3(2) (263 mg, 0.7 mmol) and (+)-camphoric acid (140 mg, 0.7 mmol) were dissolved in a mixture of methanol (1.5 mL) and acetonitrile (6 mL). The solution was refrigerated overnight and some gummy precipitate observed. The solution was concentrated to approximately half its original volume by evaporation of solvents. Ethyl acetate (2.0 mL) was added and the mixture was triturated with a spatula for approximately 15 minutes. The mixture was then slurried at room temperature overnight. White solids were collected by filtration, rinsed with ethyl acetate (3.0 mL) and dried in vacuum oven for approximately 30 minutes. (yield ˜79%).

Method 2

Tiagabine free base Form A obtained in Example 1, Method 2 (ca. 253 mg) and (+)-camphoric acid (ca. 60.2 mg) were dissolved in methanol (˜2 mL). The solution was filtered through a 0.2 μm nylon filter into another vial. Acetonitrile was added dropwise until the solution began to cloud (ca. 3 mL), and the mixture was refrigerated overnight. The resulting solid was isolated on filter paper and air dried. (Yield=ca. 194 mg, 68%).

Method 3

Tiagabine free base Form A obtained in Example 1, Method 1 (ca. 182 mg) was dissolved in dichloromethane (5 mL) and filtered (20 μm filter). The solution (50 μL) was delivered to the well in a well plate. The solvent was evaporated under high vacuum for 4 hours, producing a clear glass. A (+)-camphoratic acid solution in methanol (0.1 M, 50 μL) was added to the well. A foil seal with one pin hole per well was placed on the plate. The plate was allowed to slowly evaporate at room temperature for 48 hours. Solids that appeared crystalline by microscopy were analyzed by XRPD.

XRPD

A representative XRPD pattern of tiagabine camphorate Form A is presented in FIG. 16. Representative peaks are listed in the following Table 12.

TABLE 12
Tiagabine Camphorate Form A XRPD Peaks

| Peak No.^{a} | Position (°2θ) | d-spacing | I/Iₒ^{b} |
|---|---|---|---|
| 1 | 5.7 | 15.5 | 13 |
| 2 | 5.9 | 15.0 | 43 |
| 3 | 7.5 | 11.9 | 7 |
| 4 | 8.7 | 10.1 | 14 |
| 5 | 9.8 | 9.1 | 35 |
| 6 | 12.0 | 7.4 | 21 |
| 7 | 13.0 | 6.8 | 3 |
| 8 | 13.2 | 6.7 | 17 |
| 9 | 13.5 | 6.6 | 4 |
| 10 | 13.7 | 6.5 | 8 |
| 11 | 14.0 | 6.3 | 100 |
| 12 | 14.8 | 6.0 | 6 |
| 13 | 15.4 | 5.8 | 28 |
| 14 | 15.6 | 5.7 | 14 |
| 15 | 16.0 | 5.5 | 8 |
| 16 | 17.5 | 5.1 | 22 |
| 17 | 18.4 | 4.8 | 40 |
| 18 | 19.5 | 4.5 | 5 |
| 19 | 20.0 | 4.4 | 10 |
| 20 | 21.2 | 4.2 | 71 |
| 21 | 21.6 | 4.1 | 9 |
| 22 | 22.4 | 4.0 | 10 |
| 23 | 22.9 | 3.9 | 13 |
| 24 | 23.4 | 3.8 | 20 |
| 25 | 23.6 | 3.8 | 5 |
| 26 | 23.9 | 3.7 | 24 |
| 27 | 24.3 | 3.7 | 10 |
| 28 | 25.8 | 3.5 | 10 |
| 29 | 26.1 | 3.4 | 10 |
| 30 | 26.4 | 3.4 | 10 |
| 31 | 28.1 | 3.2 | 18 |
| 32 | 28.3 | 3.2 | 18 |
| 33 | 29.0 | 3.1 | 15 |
| 34 | 29.4 | 3.0 | 5 |
| 35 | 30.0 | 3.0 | 4 |
| 36 | 31.8-32.2^{c} | 2.8 | 13-9 |
| 37 | 32.9 | 2.7 | 7 |
| 38 | 34.4 | 2.6 | 8 |
| 39 | 35.1 | 2.6 | 7 |

^{a}Bold: Unique set of XRPD Peaks for tiagabine camphorate Form A.
^{b}Intensity of peak/Intensity of most intense peak
^{c}Broad peak - ranges are given for each parameter

DSC

DSC analysis indicated a broad major endotherm at 125° C., and a broad major endotherm at 224° C. (possible decomposition). A representative DSC curve of tiagabine camphorate Form A is presented in FIG. 17.

### Example 11

### Preparation and Characterization of Tiagabine Hydrobromide Form A

Method 1

Tiagabine obtained in Preparation 3(2) (140 mg) was dissolved in ethyl acetate (2.5 mL). The solution was filtered through a 0.2 μm nylon filter into a solution of hydrobromic acid (64 mg, ˜47%) in acetonitrile (1.5 mL). A clear solution was obtained. 2-propyl ether (2.0 mL) was added dropwise and a white precipitate was formed. The mixture was slurried at room temperature overnight. A white solid was collected by filtration and air-dried (yield˜94%).

Method 2

Tiagabine free base Form A obtained in Example 1, Method 2 (ca. 143 mg) was dissolved in a mixture of ethyl acetate and acetonitrile (3:1 (v/v), ca. 5 mL). This solution was filtered through a 0.2 μm nylon filter into another vial. Concentrated hydrobromic acid (ca. 46 mg) was dissolved in diisopropyl ether (ca. 1 mL) and carefully layered on the tiagabine free base solution. The vial was sealed and allowed to stand at room temperature overnight. The solids were filtered and air dried. (Yield=ca. 124 mg).

XRPD

A representative XRPD pattern of tiagabine hydrobromide Form A is presented in FIG. 18. Representative peaks are listed in the following Table 13.

TABLE 13
Tiagabine Hydrobromide Form A XRPD Peaks

| Peak No.^{a} | Position (°2θ) | d-spacing | I/Iₒ^{b} |
|---|---|---|---|
| 1 | 3.9 | 22.8 | 19 |
| 2 | 7.8 | 11.3 | 26 |
| 3 | 12.8 | 6.9 | 100 |
| 4 | 14.2 | 6.3 | 83 |
| 5 | 14.4 | 6.1 | 25 |
| 6 | 15.7 | 5.7 | 27 |
| 7 | 16.7 | 5.3 | 16 |
| 8 | 16.9 | 5.2 | 25 |
| 9 | 17.4 | 5.1 | 11 |
| 10 | 17.9 | 4.9 | 10 |
| 11 | 18.7 | 4.8 | 23 |
| 12 | 19.7 | 4.5 | 4 |
| 13 | 20.6 | 4.3 | 7 |
| 14 | 21.5 | 4.1 | 38 |
| 15 | 21.8 | 4.1 | 39 |
| 16 | 23.2 | 3.8 | 34 |
| 17 | 23.8 | 3.7 | 61 |
| 18 | 24.4 | 3.6 | 12 |
| 19 | 24.6 | 3.6 | 32 |
| 20 | 25.6 | 3.5 | 24 |
| 21 | 26.0 | 3.4 | 16 |
| 22 | 26.6 | 3.3 | 36 |
| 23 | 27.2 | 3.3 | 11 |
| 24 | 27.4 | 3.3 | 53 |
| 25 | 27.6 | 3.2 | 17 |
| 26 | 28.3 | 3.2 | 14 |
| 27 | 29.7 | 3.0 | 13 |
| 28 | 32.6 | 2.8 | 10 |
| 29 | 36.1 | 2.5 | 6 |

^{a}Bold: Unique set of XRPD Peaks for tiagabine hydrobromide Form A.
^{b}Intensity of peak/Intensity of most intense peak

DSC

DSC analysis indicated minor endotherms at 68° C., 100° C. (broad), 119° C., and 134° C., and a major endotherm at 165° C. A representative DSC curve of tiagabine hydrobromide Form A is presented in FIG. 19.

### Example 12

### Preparation Characterization of Tiagabine dl-Malate Form A

Method 1

Tiagabine free base Form A obtained in Example 1, Method 2 (253 mg) was dissolved in a mixture of ethyl acetate:acetonitrile (3:1 (v/v), ca. 2 mL). This solution was filtered through a 0.2 μm nylon filter into another vial.

dl-Malic acid (80 mg) was dissolved in a mixture of methanol:acetonitrile (1:1 (v/v), 3 mL). The resulting solution was added drop-wise with stirring to the solution of tiagabine free base. The combined solution was stirred for approximately one (1) hour at room temperature and solids appeared in the solution. The solution was concentrated and the resulting solids were filtered and air dried. (Yield=ca. 101 mg).

Method 2

Tiagabine free base obtained in Preparation 3(2) (270 mg, 0.7 mmol) and dl-malic acid (96 mg, 0.7 mmol) were dissolved in a mixture of methanol (1.5 mL) and acetonitrile (5 mL). The solution was refrigerated overnight. No solids were observed. The solution was concentrated to approximately half of its original volume by evaporation of solvents. Ethyl acetate (4.0 mL) was added and the mixture was slurried at room temperature overnight. Off-white solids was collected by filtration, rinsed with ethyl acetate (3.0 mL) and dried in a vacuum oven for ca. 30 min (yield ˜77%).

Method 3.

A filtered (20 μm filter) dichloromethane (5 mL) solution (50 μL) of tiagabine free base Form A obtained in Example 1, Method 1 (ca. 182 mg) was delivered to the well in a well plate. The solvent was evaporated under high vacuum for 4 hours, producing a clear glass. A dl-malic acid solution (0.1 M, 50 μL) in tetrahydrofuran/2-propanol (2:1, v/v) was added to the well. A foil seal with one pin hole per well was placed on the plate. The plate was allowed to slowly evaporate at room temperature for 48 hours.

XRPD

A representative XRPD pattern of tiagabine dl-malate Form A is presented in FIG. 20. Representative peaks are listed in the following Table 14.

TABLE 14
Tiagabine dl-Malate Form A XRPD Peaks

| Peak No.^{a} | Position (°2θ) | d-spacing | I/Iₒ^{b} |
|---|---|---|---|
| 1 | 4.2 | 21.0 | 100 |
| 2 | 11.3 | 7.8 | 38 |
| 3 | 11.9 | 7.4 | 27 |
| 4 | 12.7 | 6.9 | 8 |
| 5 | 13.5 | 6.6 | 7 |
| 6 | 15.5 | 5.7 | 28 |
| 7 | 15.9 | 5.6 | 25 |
| 8 | 16.7 | 5.3 | 29 |
| 9 | 17.0 | 5.2 | 93 |
| 10 | 18.7 | 4.7 | 23 |
| 11 | 19.2 | 4.6 | 16 |
| 12 | 21.0 | 4.2 | 39 |
| 13 | 21.4 | 4.2 | 12 |
| 14 | 23.8 | 3.7 | 38 |
| 15 | 24.2 | 3.7 | 37 |
| 16 | 24.9 | 3.6 | 27 |
| 17 | 25.1 | 3.5 | 13 |
| 18 | 25.6 | 3.5 | 14 |
| 19 | 26.8 | 3.3 | 10 |
| 20 | 27.2 | 3.3 | 8 |
| 21 | 28.0 | 3.2 | 9 |
| 22 | 32.0 | 2.8 | 7 |
| 23 | 37.1 | 2.4 | 6 |

^{a}Bold: Unique set of XRPD Peaks for tiagabine dl-malate Form A.
^{b}Intensity of peak/Intensity of most intense peak

DSC

DSC analysis indicated a major endotherm at 115° C., and a broad major endotherm at 200° C. A representative DSC curve of tiagabine dl-malate Form A is presented in FIG. 21.

### Example 13

### Preparation and Characterization of Tiagabine d-Malate Form A

Method 1

Tiagabine free base obtained in Preparation 3(2) (ca. 0.25 g) was combined with a mixture of ethyl acetate/acetonitrile (3:1 (v/v), 2 mL) with sonication. The resultant cloudy solution was filtered using a 0.2 μm filter. A mixture of methanol/acetonitrile (1:1 (v/v), 2 mL) was added dropwise with stirring. The solution was stirred for approximately 1 hr and then left uncovered overnight, resulting in a gummy residue. To the residue was added a mixture of ethyl acetate/acetonitrile (3:1 (v/v), 700 μL) with stirring. The mixture was left at room temperature overnight, then refrigerated for one day, then placed in a freezer for 6 days, after which the solvent was allowed to evaporate at ambient conditions. The resulting brown solids were slurried in 1 mL of ether for one day before collected by vacuum filtration.

Method 2

Tiagabine free base Form A obtained in Example 1, Method 2 (ca. 253 mg) was dissolved in a mixture of ethyl acetate:acetonitrile (3:1 (v/v), 2 mL). This solution was filtered through a 0.2 μm nylon filter into another vial.

A solution of d-malic acid (ca. 80 mg) in a mixture of methanol:acetonitrile (1:1 (v/v), 2 mL) was added drop-wise with stirring to the solution of tiagabine free base. The resulting solution was stirred for approximately one (1) hour at room temperature and solids appeared in the solution. The solution was concentrated and the resulting solids were filtered and air dried. (Yield=ca. 97 mg, 32%).

XRPD

A representative XRPD pattern of tiagabine d-malate Form A is presented in FIG. 22. Representative peaks are listed in the following Table 15.

TABLE 15
Tiagabine d-Malate Form A XRPD Peaks

| Peak No.^{a} | Position (°2θ) | d-spacing | I/Iₒ^{b} |
|---|---|---|---|
| 1 | 4.2 | 21.0 | 31 |
| 2 | 11.3 | 7.8 | 22 |
| 3 | 11.9 | 7.4 | 15 |
| 4 | 12.8 | 6.9 | 6 |
| 5 | 13.5 | 6.6 | 8 |
| 6 | 15.5 | 5.7 | 20 |
| 7 | 15.9 | 5.6 | 19 |
| 8 | 17.0 | 5.2 | 100 |
| 9 | 18.7 | 4.7 | 24 |
| 10 | 19.2 | 4.6 | 16 |
| 11 | 21.1 | 4.2 | 37 |
| 12 | 21.4 | 4.1 | 11 |
| 13 | 22.0 | 4.0 | 3 |
| 14 | 23.8 | 3.7 | 44 |
| 15 | 24.2 | 3.7 | 42 |
| 16 | 24.6 | 3.6 | 29 |
| 17 | 25.0 | 3.6 | 18 |
| 18 | 25.6 | 3.5 | 12 |
| 19 | 28.0 | 3.2 | 8 |
| 20 | 28.2 | 3.2 | 7 |
| 21 | 30.6 | 2.9 | 7 |
| 22 | 31.9 | 2.8 | 6 |
| 23 | 34.1 | 2.6 | 7 |
| 24 | 34.6 | 2.6 | 7 |
| 25 | 34.8 | 2.6 | 3 |
| 26 | 35.6 | 2.5 | 13 |
| 27 | 36.2 | 2.5 | 5 |
| 28 | 37.2 | 2.4 | 9 |

^{a}Bold: Unique set of XRPD Peaks for tiagabine d-malate Form A.
^{b}Intensity of peak/Intensity of most intense peak

DSC

DSC analysis indicated a major endotherm at 121° C. and a broad major endotherm at 200° C. A representative DSC curve of tiagabine d-malate Form A is presented in FIG. 23.

### Example 14

### Preparation and Characterization of Tiagabine Tartrate Form A

Method 1

Tiagabine free base obtained in Preparation 3(2) (264 mg, 0.7 mmol) and tartaric acid (105 mg, 0.7 mmol) were dissolved in a mixture of methanol (1.5 mL) and acetonitrile (3 mL). The solution was refrigerated overnight, giving a cloudy solution. The solution was concentrated to approximately half of its original volume by evaporation of solvents. Ethyl acetate (2.0 mL) was added and the mixture was slurried at room temperature overnight. White solids were collected by filtration, rinsed with ethyl acetate (3.0 mL) and dried in a vacuum oven for ca. 30 minutes (yield=ca. 91%).

Method 2

Tiagabine free base Form A obtained in Example 1, Method 2 (ca. 253 mg) and L-(+)-tartaric acid (ca. 45 mg) were dissolved in methanol (ca. 5 mL). The solution was filtered through a 0.2 μm nylon filter into a vial. Acetonitrile (ca. 3 mL) was added and the resulting solution was allowed to evaporate slowly at room temperature until the solution volume was reduced to approximately 3 mL. The resulting solids were isolated on filter paper and air dried. (Yield=ca. 230 mg).

Method 3

A filtered (20 μm filter) dichloromethane (5 mL) solution (50 μL) of tiagabine free base Form A obtained in Example 1, Method 1 (ca. 182 mg) was delivered to the well in a well plate. The solvent was evaporated under high vacuum for 4 hours, producing a clear glass. A L (+)-tartaric acid solution (0.1 M, 50 μL) in acetone/ethyl acetate (1:1, v/v) was added to the well. A foil seal with one pin hole per well was placed on the plate. The plate was allowed to slowly evaporate at room temperature for 48 hours.

Method 4

A filtered (20 μm filter) dichloromethane (5 mL) solution (50 μL) of tiagabine free base Form A obtained in Example 1, Method 1 (ca. 182 mg) was delivered to the well in a well plate. The solvent was evaporated under high vacuum for 4 hours, producing a clear glass. A dl-tartaric acid solution (0.1 M, 50 μL) in tetrahydrofuran/2-propanol (2:1, v/v) was added to the well. A foil seal with one pin hole per well was placed on the plate. The plate was allowed to slowly evaporate at room temperature for 48 hours.

XRPD

A representative XRPD pattern of tiagabine tartrate Form A is presented in FIG. 24. Representative peaks are listed in the following Table 16.

TABLE 16
Tiagabine Tartrate Form A XRPD Peaks

| Peak No.^{a} | Position (°2θ) | d-spacing | I/Iₒ^{b} |
|---|---|---|---|
| 1 | 4.1 | 21.4 | 62 |
| 2 | 8.4 | 10.5 | 10 |
| 3 | 11.5 | 7.7 | 14 |
| 4 | 11.9 | 7.4 | 6 |
| 5 | 12.6 | 7.0 | 54 |
| 6 | 13.3 | 6.6 | 40 |
| 7 | 13.6 | 6.5 | 100 |
| 8 | 16.0 | 5.5 | 20 |
| 9 | 16.5 | 5.4 | 79 |
| 10 | 16.7 | 5.3 | 53 |
| 11 | 17.0 | 5.2 | 45 |
| 12 | 17.9 | 5.0 | 23 |
| 13 | 18.8 | 4.7 | 10 |
| 14 | 19.0 | 4.7 | 22 |
| 15 | 20.3 | 4.4 | 35 |
| 16 | 21.5 | 4.1 | 30 |
| 17 | 22.3 | 4.0 | 8 |
| 18 | 23.1 | 3.9 | 15 |
| 19 | 23.9 | 3.7 | 22 |
| 20 | 24.6 | 3.6 | 67 |
| 21 | 24.8 | 3.6 | 15 |
| 22 | 25.2 | 3.5 | 13 |
| 23 | 25.5 | 3.5 | 15 |
| 24 | 26.0 | 3.4 | 15 |
| 25 | 26.7 | 3.3 | 16 |
| 26 | 27.3 | 3.3 | 13 |
| 27 | 28.0 | 3.2 | 20 |
| 28 | 28.6 | 3.1 | 7 |
| 29 | 29.9 | 3.0 | 15 |
| 30 | 32.3 | 2.8 | 8 |
| 31 | 35.7 | 2.5 | 12 |
| 32 | 39.5 | 2.3 | 5 |

^{a}Bold: Unique set of XRPD Peaks for tiagabine tartrate Form A.
^{b}Intensity of peak/Intensity of most intense peak

DSC

DSC analysis indicated a minor endotherm at 138° C., a minor exotherm at 142° C., and a major endotherm at 162° C. A representative DSC curve of tiagabine tartrate Form A is presented in FIG. 25.

### Example 15

### Preparation and Characterization of Tiagabine Hydrochloride Cocrystal with 2-Furancarboxylic Acid

Method 1

Tiagabine hydrochloride monohydrate (0.0863 grams), 2-furancarboxylic acid (0.0226 grams) and methanol (1 drop) were charged to an agate lined canister. The mixture was processed using an agate ball mill for approximately 2 minutes using a Retsch mm200 milling apparatus set at 30 Hz. The solids were scraped from the sides of the canister and milled for an additional 4 minutes at 30 Hz.

Method 2

Tiagabine hydrochloride monohydrate (ca. 58 mg) and 2-furancarboxylic acid (ca. 15 mg) were processed using an agate ball mill for approximately 5 minutes using a Retsch mm200 milling apparatus. Approximately 56 mg of solid was isolated from the grinding jar.

XRPD

A representative XRPD pattern of tiagabine hydrochloride cocrystal with 2-furancarboxylic acid is presented in FIG. 26. Representative peaks are listed in the following Table 17.

TABLE 17
Tiagabine Hydrochloride Cocrystal with 2-Furancarboxylic acid XRPD
Peaks

| Peak No.^{a} | Position (°2θ) | d-spacing | I/Iₒ^{b} |
|---|---|---|---|
| 1 | 7.5 | 11.9 | 29 |
| 2 | 11.6 | 7.6 | 21 |
| 3 | 14.7 | 6.0 | 81 |
| 4 | 14.9 | 5.9 | 30 |
| 5 | 15.6 | 5.7 | 14 |
| 6 | 15.7 | 5.6 | 23 |
| 7 | 16.4 | 5.4 | 7 |
| 8 | 16.6 | 5.3 | 54 |
| 9 | 17.2 | 5.2 | 100 |
| 10 | 17.6 | 5.0 | 10 |
| 11 | 19.0 | 4.7 | 48 |
| 12 | 19.4 | 4.6 | 3 |
| 13 | 20.2 | 4.4 | 10 |
| 14 | 20.3 | 4.4 | 9 |
| 15 | 21.0 | 4.2 | 58 |
| 16 | 21.3 | 4.2 | 18 |
| 17 | 21.7 | 4.1 | 87 |
| 18 | 22.1 | 4.0 | 12 |
| 19 | 22.9 | 3.9 | 88 |
| 20 | 23.2 | 3.8 | 11 |
| 21 | 24.8 | 3.6 | 25 |
| 22 | 25.1 | 3.6 | 34 |
| 23 | 25.5 | 3.5 | 83 |
| 24 | 25.8 | 3.5 | 43 |
| 25 | 26.1 | 3.4 | 16 |
| 26 | 26.3 | 3.4 | 16 |
| 27 | 26.6 | 3.3 | 92 |
| 28 | 27.0 | 3.3 | 28 |
| 29 | 27.7 | 3.2 | 27 |
| 30 | 28.0 | 3.2 | 31 |
| 31 | 29.3 | 3.1 | 28 |
| 32 | 29.6 | 3.0 | 14 |
| 33 | 30.0 | 3.0 | 5 |
| 34 | 30.6 | 2.9 | 15 |
| 35 | 30.9 | 2.9 | 7 |
| 36 | 31.1 | 2.9 | 17 |
| 37 | 31.4 | 2.8 | 11 |
| 38 | 33.7 | 2.7 | 8 |
| 39 | 34.1 | 2.6 | 16 |
| 40 | 34.9 | 2.6 | 17 |
| 41 | 35.1 | 2.6 | 18 |
| 42 | 35.8 | 2.5 | 5 |

^{a}Bold: Unique set of XRPD Peaks for tiagabine hydrochloride cocrystal with 2-furancarboxylic acid.
^{b}Intensity of peak/Intensity of most intense peak

DSC

DSC analysis indicated a major endotherm at 119° C. A representative DSC curve of tiagabine hydrochloride cocrystal with 2-furancarboxylic acid is presented in FIG. 27.

### Example 16

### Preparation and Characterization of Tiagabine Hydrochloride Form G

182 mg of tiagabine free base was dissolved in 5 mL dichloromethane. Approximately 50 μL of the resulting solution was delivered to the well of a well plate. The solvent was evaporated under high vacuum for 4 hours, producing a clear glass. Chloroform (approximately 50 μL) was added to the well and the solution reacted with 50 μL of 0.1M HCl solution in methanol. The plate was sealed and stored at 3° C. for 24 hours after which time solids were precipitated with cyclohexane (30 μL). The plate was store at 3° C. for 24 hours and then the solvent allowed to slowly evaporate at room temperature.

XRPD

A representative XRPD pattern of tiagabine hydrochloride Form G is presented in FIG. 28. Representative peaks are listed in the following Table 18.

TABLE 18
Tiagabine HCl Form G XRPD Peaks

| Peak No.^{a} | Position (°2θ) | d-spacing | Intensity | I/Iₒ^{b} |
|---|---|---|---|---|
| 1 | 3.9 | 22.8 | 6290 | 20 |
| 2 | 12.6 | 7.0 | 3536 | 12 |
| 3 | 14.7 | 6.0 | 14105 | 46 |
| 4 | 15.3 | 5.8 | 4476 | 15 |
| 5 | 16.0 | 5.5 | 6206 | 20 |
| 6 | 16.9 | 5.2 | 30732 | 100 |
| 7 | 17.8 | 5.0 | 3365 | 11 |
| 8 | 18.5 | 4.8 | 5290 | 17 |
| 9 | 19.2 | 4.6 | 5250 | 17 |
| 10 | 20.5 | 4.3 | 10371 | 34 |
| 11 | 21.5 | 4.1 | 5628 | 18 |
| 12 | 22.4 | 4.0 | 6930 | 23 |
| 13 | 22.9 | 3.9 | 9982 | 32 |
| 14 | 23.7 | 3.8 | 5386 | 18 |
| 15 | 24.8 | 3.6 | 4350 | 14 |
| 16 | 25.5 | 3.5 | 16555 | 54 |
| 17 | 26.3 | 3.4 | 8537 | 28 |
| 18 | 27.0 | 3.3 | 8004 | 26 |
| 19 | 28.1 | 3.2 | 10194 | 33 |
| 20 | 28.7 | 3.1 | 4784 | 16 |
| 21 | 30.9 | 2.9 | 3641 | 12 |

^{a}Bold: Unique set of XRPD Peaks for Form G.
^{b}Intensity of peak/Intensity of most intense peak × 100

### Example 17

### Preparation and Characterization of Tiagabine Hydrochloride Form K

Preparation Method 1

150 mg of tiagabine HCl monohydrate was dissolved in 1.25 mL of chloroform to give clear solution. Approximately 0.25 mL of heptane was added to the solution and a white precipitation was formed. The mixture was slurried at ambient temperature overnight. The liquid was decanted and the remaining solids were air dried.

Preparation Method 2

A mixture of 89 mg of tiagabine HCl monohydrate and 4 mL of chloroform was slurried for 4 days at room temperature. The white solids were collected by filtration and air dried.

Preparation Method 3

Amorphous tiagabine HCl (29 mg) was dissolved in 50 [L of chloroform. Solids precipitated and the solvent evaporated under a gentle stream of nitrogen. The sample was stored in a freezer inside a desiccator prior to XRPD analysis.

XRPD

A representative XRPD pattern of tiagabine hydrochloride Form K is presented in FIG. 29. Representative peaks are listed in the following Table 19.

TABLE 19
Tiagabine HCl Form K XRPD Peaks

| Peak No.^{a} | Position (°2θ) | d-spacing | Intensity | I/Iₒ^{b} |
|---|---|---|---|---|
| 1 | 5.7 | 15.5 | 181 | 21 |
| 2 | 6.4 | 13.8 | 65 | 8 |
| 3 | 8.4 | 10.5 | 68 | 8 |
| 4 | 11.4 | 7.8 | 49 | 6 |
| 5 | 13.0 | 6.8 | 91 | 11 |
| 6 | 13.3 | 6.6 | 277 | 32 |
| 7 | 14.8 | 6.0 | 161 | 19 |
| 8 | 15.3 | 5.8 | 86 | 10 |
| 9 | 16.6 | 5.3 | 855 | 100 |
| 10 | 17.1 | 5.2 | 118 | 14 |
| 11 | 19.3 | 4.6 | 60 | 7 |
| 12 | 19.6 | 4.5 | 53 | 6 |
| 13 | 20.1 | 4.4 | 129 | 15 |
| 14 | 20.6 | 4.3 | 186 | 22 |
| 15 | 22.6 | 3.9 | 110 | 13 |
| 16 | 23.0 | 3.9 | 174 | 20 |
| 17 | 23.6 | 3.8 | 260 | 30 |
| 18 | 24.5 | 3.6 | 348 | 41 |
| 19 | 24.9 | 3.6 | 406 | 47 |
| 20 | 25.3 | 3.5 | 191 | 22 |
| 21 | 25.9 | 3.4 | 104 | 12 |
| 22 | 26.6 | 3.4 | 57 | 7 |
| 23 | 26.9 | 3.3 | 78 | 9 |
| 24 | 28.1 | 3.2 | 52 | 6 |
| 25 | 29.1 | 3.1 | 78 | 9 |
| 26 | 29.8 | 3.0 | 52 | 6 |
| 27 | 31.1 | 2.9 | 50 | 6 |
| 28 | 33.6 | 2.7 | 65 | 8 |
| 29 | 34.6 | 2.6 | 81 | 9 |
| 30 | 35.4 | 2.5 | 52 | 6 |

^{a}Bold: Unique set of XRPD Peaks for Form K.
^{b}Intensity of peak/Intensity of most intense peak × 100

TGA

TGA analysis indicated a 16.9% weight loss between 25 to 150° C.

¹H NMR

¹H NMR analysis indicated that the tiagabine hydrochloride Form K contained 0.34 moles of chloroform per mole of tiagabine HCl.

Stability

Tiagabine HCl Form K was stored for approximately two months under conditions of ambient temperature and humidity. XRPD analysis of the resulting sample indicated a mixture of tiagabine HCl Forms Q and B.

### Example 18

### Preparation and Characterization of Tiagabine Hydrochloride Form L

Preparation Method 1

Approximately 92 mg of tiagabine HCl monohydrate was dissolved in approximately 2 mL of nitromethane. A clear solution was obtained at first and solid quickly precipitated out. The sample was capped and placed in a vacuum hood at ambient temperature overnight. The liquid was decanted and the remaining solids were air dried.

Preparation Method 2

A saturated solution of tiagabine HCl monohydrate in nitromethane was filtered through a 0.2 μm nylon filter into a vial. The resulting solution in an open vial was allowed to evaporate quickly until dryness. A white, needle-like, solid was obtained.

XRPD

A representative XRPD pattern of tiagabine hydrochloride Form L is presented in FIG. 30. Representative peaks are listed in the following Table 20.

TABLE 20
Tiagabine HCl Form L XRPD Peaks

| Peak No.^{a} | Position (°2θ) | d-spacing | Intensity | I/Iₒ^{b} |
|---|---|---|---|---|
| 1 | 7.7 | 11.4 | 352 | 24 |
| 2 | 12.5 | 7.1 | 1489 | 100 |
| 3 | 12.8 | 6.9 | 278 | 19 |
| 4 | 13.2 | 6.7 | 424 | 28 |
| 5 | 13.5 | 6.5 | 134 | 9 |
| 6 | 14.2 | 6.2 | 129 | 9 |
| 7 | 14.5 | 6.1 | 768 | 52 |
| 8 | 15.4 | 5.7 | 153 | 10 |
| 9 | 16.6 | 5.3 | 378 | 25 |
| 10 | 16.9 | 5.2 | 733 | 49 |
| 11 | 17.1 | 5.2 | 937 | 63 |
| 12 | 17.5 | 5.1 | 335 | 22 |
| 13 | 18.0 | 4.9 | 371 | 25 |
| 14 | 18.2 | 4.9 | 117 | 8 |
| 15 | 18.8 | 4.7 | 326 | 22 |
| 16 | 21.1 | 4.2 | 1109 | 74 |
| 17 | 21.8 | 4.1 | 761 | 51 |
| 18 | 23.1 | 3.8 | 387 | 26 |
| 19 | 24.0 | 3.7 | 526 | 35 |
| 20 | 24.2 | 3.7 | 211 | 14 |
| 21 | 24.6 | 3.6 | 1052 | 71 |
| 22 | 24.9 | 3.6 | 201 | 13 |
| 23 | 25.1 | 3.5 | 770 | 52 |
| 24 | 26.2 | 3.4 | 672 | 45 |
| 25 | 26.6 | 3.3 | 145 | 10 |
| 26 | 27.5 | 3.2 | 433 | 29 |
| 27 | 28.0 | 3.2 | 615 | 41 |
| 28 | 29.8 | 3.0 | 151 | 10 |
| 29 | 30.7 | 2.9 | 148 | 10 |
| 30 | 37.3 | 2.4 | 132 | 9 |

^{a}Bold: Unique set of XRPD Peaks for Form L.
^{b}Intensity of peak/Intensity of most intense peak × 100

Stability

Tiagabine HCl Form L was stored for approximately two months under conditions of ambient temperature and humidity. XRPD analysis of the resulting sample indicated a mixture of tiagabine HCl Forms B and Q.

### Example 19

### Preparation and Characterization of Tiagabine Hydrochloride Form N

A mixture of 22 mg of tiagabine HCl amorphous and about 1.5 mL of benzonitrile was warmed in a sand bath to give a clear solution. After several hours, a precipitate was formed. The solids were collected by filtration and dried under a gentle stream of nitrogen.

XRPD

A representative XRPD pattern of tiagabine hydrochloride Form N is presented in FIG. 31. Representative peaks are listed in the following Table 21.

TABLE 21
Tiagabine HCl Form N XRPD Peaks

| Peak No.^{a} | Position (°2θ) | d-spacing | Intensity | I/Iₒ^{b} |
|---|---|---|---|---|
| 1 | 7.1 | 12.4 | 72 | 9 |
| 2 | 9.8 | 9.0 | 79 | 10 |
| 3 | 11.9 | 7.4 | 204 | 27 |
| 4 | 12.6 | 7.0 | 92 | 12 |
| 5 | 14.1 | 6.3 | 610 | 80 |
| 6 | 14.5 | 6.1 | 536 | 70 |
| 7 | 14.7 | 6.0 | 166 | 22 |
| 8 | 15.6 | 5.7 | 762 | 100 |
| 9 | 16.6 | 5.3 | 145 | 19 |
| 10 | 17.1 | 5.2 | 644 | 85 |
| 11 | 17.9 | 5.0 | 101 | 13 |
| 12 | 18.4 | 4.8 | 142 | 19 |
| 13 | 18.8 | 4.7 | 83 | 11 |
| 14 | 19.0 | 4.7 | 77 | 10 |
| 15 | 19.6 | 4.5 | 408 | 54 |
| 16 | 20.0 | 4.4 | 104 | 14 |
| 17 | 20.7 | 4.3 | 73 | 10 |
| 18 | 21.4 | 4.2 | 232 | 30 |
| 19 | 21.7 | 4.1 | 91 | 12 |
| 20 | 22.6 | 3.9 | 386 | 51 |
| 21 | 23.2 | 3.8 | 227 | 30 |
| 22 | 23.8 | 3.7 | 212 | 28 |
| 23 | 24.7 | 3.6 | 695 | 91 |
| 24 | 25.0 | 3.6 | 368 | 48 |
| 25 | 25.6 | 3.5 | 219 | 29 |
| 26 | 26.0 | 3.4 | 70 | 9 |
| 27 | 26.2 | 3.4 | 124 | 16 |
| 28 | 26.5 | 3.4 | 83 | 11 |
| 29 | 26.9 | 3.3 | 142 | 19 |
| 30 | 27.4 | 3.3 | 149 | 20 |
| 31 | 27.9 | 3.2 | 213 | 28 |

^{a}Bold: Unique set of XRPD Peaks for Form N.
^{b}Intensity of peak/Intensity of most intense peak × 100

TGA

TGA analysis indicated a 10.6% weight loss between 25 to 125° C.

¹H NMR

¹H NMR analysis indicated that the tiagabine hydrochloride Form N contained 2.6 moles of benzonitrile per mole of tiagabine HCl.

### Example 20

### Preparation and Characterization of Tiagabine Hydrochloride Form O

A small amount of tiagabine HCl monohydrate was heated on a XRPD sample holder to 140° C. An XRPD pattern was recorded at 140° C.

XRPD

A representative XRPD pattern of tiagabine hydrochloride Form O is presented in FIG. 32. Representative peaks are listed in the following Table 22.

TABLE 22
Tiagabine HCl Form O XRPD Peaks

| Peak No.^{a} | Position (°2θ) | d-spacing | Intensity | I/Iₒ^{b} |
|---|---|---|---|---|
| 1 | 12.6 | 7.0 | 27 | 34 |
| 2 | 13.3 | 6.7 | 17 | 22 |
| 3 | 14.2 | 6.2 | 19 | 24 |
| 4 | 14.6 | 6.1 | 39 | 49 |
| 5 | 14.8 | 6.0 | 16 | 20 |
| 6 | 15.7 | 5.7 | 22 | 28 |
| 7 | 16.0 | 5.5 | 31 | 39 |
| 8 | 16.4 | 5.4 | 40 | 51 |
| 9 | 16.9 | 5.3 | 9 | 11 |
| 10 | 18.6 | 4.8 | 54 | 68 |
| 11 | 18.9 | 4.7 | 79 | 100 |
| 12 | 20.6 | 4.3 | 18 | 23 |
| 13 | 22.0 | 4.0 | 21 | 27 |
| 14 | 22.3 | 4.0 | 10 | 13 |
| 15 | 22.6 | 3.9 | 12 | 15 |
| 16 | 23.3 | 3.8 | 52 | 66 |
| 17 | 23.6 | 3.8 | 33 | 42 |
| 18 | 24.3 | 3.7 | 52 | 66 |
| 19 | 24.7 | 3.6 | 37 | 47 |
| 20 | 25.6 | 3.5 | 17 | 22 |
| 21 | 25.9 | 3.4 | 51 | 65 |
| 22 | 26.5 | 3.4 | 15 | 19 |
| 23 | 26.8 | 3.3 | 17 | 22 |
| 24 | 28.7 | 3.1 | 12 | 15 |
| 25 | 32.6 | 2.7 | 8 | 10 |
| 26 | 33.4 | 2.7 | 11 | 14 |
| 27 | 34.5 | 2.6 | 15 | 19 |
| 28 | 35.4 | 2.5 | 16 | 20 |

^{a}Bold: Unique set of XRPD Peaks for Form O.
^{b}Intensity of peak/Intensity of most intense peak × 100

### Example 21

### Preparation and Characterization of Tiagabine Hydrochloride Form R

A mixture of 178 mg of tiagabine HCl monohydrate and 4 mL of nitromethane was slurried for 4 days at room temperature. The white solids were collected by filtration and dried in the air.

XRPD

A representative XRPD pattern of tiagabine hydrochloride Form R is presented in FIG. 33. Representative peaks are listed in the following Table 23.

TABLE 23
Tiagabine HCl Form R XRPD Peaks

| Peak No.^{a} | Position (°2θ) | d-spacing | Intensity | I/Iₒ^{b} |
|---|---|---|---|---|
| 1 | 10.8 | 8.2 | 44 | 21 |
| 2 | 12.3 | 7.2 | 85 | 41 |
| 3 | 12.8 | 6.9 | 35 | 17 |
| 4 | 13.0 | 6.8 | 139 | 68 |
| 5 | 13.2 | 6.7 | 43 | 21 |
| 6 | 13.5 | 6.6 | 155 | 76 |
| 7 | 15.1 | 5.9 | 39 | 19 |
| 8 | 15.3 | 5.8 | 140 | 68 |
| 9 | 16.4 | 5.4 | 30 | 15 |
| 10 | 16.7 | 5.3 | 147 | 72 |
| 11 | 17.8 | 5.0 | 156 | 76 |
| 12 | 18.0 | 4.9 | 33 | 16 |
| 13 | 18.6 | 4.8 | 18 | 9 |
| 14 | 19.4 | 4.6 | 20 | 10 |
| 15 | 19.7 | 4.5 | 107 | 52 |
| 16 | 19.8 | 4.5 | 128 | 62 |
| 17 | 20.9 | 4.3 | 36 | 18 |
| 18 | 21.2 | 4.2 | 113 | 55 |
| 19 | 21.4 | 4.1 | 55 | 27 |
| 20 | 22.2 | 4.0 | 95 | 46 |
| 21 | 22.9 | 3.9 | 37 | 18 |
| 22 | 23.7 | 3.8 | 67 | 33 |
| 23 | 25.0 | 3.6 | 82 | 40 |
| 24 | 25.2 | 3.5 | 89 | 43 |
| 25 | 25.4 | 3.5 | 205 | 100 |
| 26 | 25.8 | 3.5 | 187 | 91 |
| 27 | 26.1 | 3.4 | 139 | 68 |
| 28 | 26.9 | 3.3 | 96 | 47 |
| 29 | 28.0 | 3.2 | 127 | 62 |
| 30 | 28.6 | 3.1 | 32 | 16 |
| 31 | 29.0 | 3.1 | 94 | 46 |
| 32 | 29.1 | 3.1 | 54 | 26 |
| 33 | 30.3 | 2.9 | 47 | 23 |
| 34 | 32.2 | 2.8 | 69 | 34 |
| 35 | 33.4 | 2.7 | 31 | 15 |
| 36 | 36.0 | 2.5 | 35 | 17 |
| 37 | 36.2 | 2.5 | 38 | 19 |
| 38 | 39.7 | 2.3 | 25 | 12 |

^{a}Bold: Unique set of XRPD Peaks for Form R
^{b}Intensity of peak/Intensity of most intense peak × 100

TGA

TGA analysis indicated a 9.9% weight loss between 25 to 150° C.

¹H NMR

¹NMR analysis indicated that the tiagabine hydrochloride Form R contained 0.57 moles of nitromethane per mole of tiagabine HCl.

### Example 22

### Preparation and Characterization of Tiagabine Hydrochloride Form U

A mixture of 105 mg of tiagabine HCl monohydrate and 5 mL of 1,2-dichloroethane was slurried at room temperature for 3 days. The resulting solids were collected by filtration and dried in the air.

XRPD

A representative XRPD pattern of tiagabine hydrochloride Form U is presented in FIG. 34. Representative peaks are listed in the following Table 24.

TABLE 24
Tiagabine HCl Form U XRPD Peaks

| | Position | | | |
|---|---|---|---|---|
| Peak No.^{a} | (°2θ) | d-spacing | Intensity | I/Iₒ^{b} |
| 1 | 7.9 | 11.2 | 12 | 9 |
| 2 | 11.9 | 7.5 | 16 | 12 |
| 3 | 12.6 | 7.0 | 28 | 20 |
| 4 | 14.4 | 6.2 | 50 | 36 |
| 5 | 15.9 | 5.6 | 19 | 14 |
| 6 | 16.4 | 5.4 | 115 | 84 |
| 7 | 16.9 | 5.2 | 52 | 38 |
| 8 | 17.5 | 5.1 | 23 | 17 |
| 9 | 18.3 | 4.9 | 36 | 26 |
| 10 | 19.2 | 4.6 | 14 | 10 |
| 11 | 19.5 | 4.6 | 39 | 28 |
| 12 | 19.9 | 4.5 | 40 | 29 |
| 13 | 20.3 | 4.4 | 14 | 10 |
| 14 | 21.2 | 4.2 | 108 | 79 |
| 15 | 21.6 | 4.1 | 119 | 87 |
| 16 | 22.5 | 3.9 | 28 | 20 |
| 17 | 22.9 | 3.9 | 94 | 69 |
| 18 | 23.3 | 3.8 | 19 | 14 |
| 19 | 23.9 | 3.7 | 137 | 100 |
| 20 | 24.2 | 3.7 | 23 | 17 |
| 21 | 24.7 | 3.6 | 26 | 19 |
| 22 | 25.1 | 3.5 | 22 | 16 |
| 23 | 25.4 | 3.5 | 18 | 13 |
| 24 | 25.6 | 3.5 | 26 | 19 |
| 25 | 25.9 | 3.4 | 43 | 31 |
| 26 | 26.6 | 3.4 | 91 | 66 |
| 27 | 26.8 | 3.3 | 41 | 30 |
| 28 | 27.4 | 3.3 | 65 | 47 |
| 29 | 27.6 | 3.2 | 99 | 72 |
| 30 | 28.4 | 3.1 | 17 | 12 |
| 31 | 28.7 | 3.1 | 16 | 12 |
| 32 | 29.3 | 3.1 | 23 | 17 |
| 33 | 30.2 | 3.0 | 18 | 13 |
| 34 | 31.5 | 2.8 | 20 | 15 |
| 35 | 32.2 | 2.8 | 19 | 14 |
| 36 | 32.7 | 2.7 | 12 | 9 |
| 37 | 34.0 | 2.6 | 15 | 11 |
| 38 | 36.3 | 2.5 | 23 | 17 |
| 39 | 38.3 | 2.4 | 28 | 20 |
| 40 | 39.1 | 2.3 | 15 | 11 |

^{a}Bold: Unique set of XRPD Peaks for Form U
^{b}Intensity of peak/Intensity of most intense peak × 100

TGA

TGA analysis indicated a two step weight loss of 1.8% between 18 and 60° C. and 11% between 60 and 130° C.

¹H NMR

¹H NMR analysis indicated that the tiagabine hydrochloride Form U contained 0.47 moles of 1,2-dichloroethane per mole of tiagabine HCl.

### Example 23

### Preparation and Characterization of Tiagabine Hydrochloride Form V

A mixture of 120 mg of tiagabine HCl monohydrate and 5 mL of 1,2-dimethoxyethane was slurried at room temperature for 3 days. The resulting solids were collected by filtration and dried in the air.

XRPD

A representative XRPD pattern of tiagabine hydrochloride Form V is presented in FIG. 35. Representative peaks are listed in the following Table 25.

TABLE 25
Tiagabine HCl Form V XRPD Peaks

| Peak | | | | |
|---|---|---|---|---|
| No.^{a} | Position (°2θ) | d-spacing | Intensity | I/Iₒ^{b} |
| 1 | 5.7 | 15.4 | 13 | 8 |
| 2 | 6.4 | 13.7 | 13 | 8 |
| 3 | 7.4 | 11.9 | 66 | 40 |
| 4 | 7.7 | 11.4 | 29 | 18 |
| 5 | 10.9 | 8.1 | 20 | 12 |
| 6 | 11.2 | 7.9 | 35 | 21 |
| 7 | 11.6 | 7.7 | 73 | 44 |
| 8 | 12.9 | 6.9 | 80 | 48 |
| 9 | 13.5 | 6.6 | 14 | 8 |
| 10 | 13.7 | 6.4 | 37 | 22 |
| 11 | 14.9 | 5.9 | 29 | 18 |
| 12 | 15.5 | 5.7 | 54 | 33 |
| 13 | 15.8 | 5.6 | 109 | 66 |
| 14 | 16.1 | 5.5 | 165 | 100 |
| 15 | 16.5 | 5.4 | 37 | 22 |
| 16 | 17.8 | 5.0 | 15 | 9 |
| 17 | 18.5 | 4.8 | 153 | 93 |
| 18 | 19.4 | 4.6 | 112 | 68 |
| 19 | 20.9 | 4.3 | 27 | 16 |
| 20 | 21.2 | 4.2 | 140 | 85 |
| 21 | 21.5 | 4.1 | 33 | 20 |
| 22 | 21.9 | 4.1 | 21 | 13 |
| 23 | 22.4 | 4.0 | 29 | 18 |
| 24 | 23.0 | 3.9 | 28 | 17 |
| 25 | 23.6 | 3.8 | 40 | 24 |
| 26 | 23.9 | 3.7 | 162 | 98 |
| 27 | 25.2 | 3.5 | 62 | 38 |
| 28 | 25.9 | 3.4 | 31 | 19 |
| 29 | 26.1 | 3.4 | 17 | 10 |
| 30 | 26.4 | 3.4 | 73 | 44 |
| 31 | 26.7 | 3.3 | 27 | 16 |
| 32 | 27.7 | 3.2 | 16 | 10 |
| 33 | 28.0 | 3.2 | 18 | 11 |
| 34 | 28.7 | 3.1 | 12 | 7 |
| 35 | 29.6 | 3.0 | 19 | 12 |
| 36 | 32.6 | 2.7 | 19 | 12 |
| 37 | 33.4 | 2.7 | 28 | 17 |
| 38 | 34.3 | 2.6 | 15 | 9 |
| 39 | 35.2 | 2.5 | 24 | 15 |
| 40 | 36.6 | 2.5 | 26 | 16 |
| 41 | 37.6 | 2.4 | 23 | 14 |
| 42 | 39.5 | 2.3 | 18 | 11 |

^{a}Bold: Unique set of XRPD Peaks for Form V
^{b}Intensity of peak/Intensity of most intense peak × 100

### Example 24

### Preparation and Characterization of Tiagabine Hydrochloride Form AC

Preparation Method 1

Approximately 120 mg of tiagabine HCl monohydrate was dissolved in approximately 2 mL of cyclohexanol. A clear solution was observed at first and solid quickly precipitate out. The sample was capped and placed in a vacuum hood at ambient temperature for 3 days. The resulting solids were collected by filtration and dried in the air.

Preparation Method 2

Tiagabine HCl monohydrate (120 mg) in cyclohexanol (2.0 mL) was slurried for 3 days and filtered through 0.2 μm nylon filter. The filtrate was allowed to evaporate under ambient conditions. An off-white solid was obtained.

XRPD

A representative XRPD pattern of tiagabine hydrochloride Form AC is presented in FIG. 36. Representative peaks are listed in the following Table 26.

TABLE 26
Tiagabine HCl Form AC XRPD Peaks

| Peak | | | | |
|---|---|---|---|---|
| No.^{a} | Position (°2θ) | d-spacing | Intensity | I/Iₒ^{b} |
| 1 | 7.8 | 11.3 | 19 | 12 |
| 2 | 8.5 | 10.3 | 19 | 12 |
| 3 | 11.0 | 8.1 | 15 | 10 |
| 4 | 12.4 | 7.1 | 36 | 23 |
| 5 | 13.7 | 6.5 | 8 | 5 |
| 6 | 14.7 | 6.0 | 36 | 23 |
| 7 | 15.3 | 5.8 | 52 | 33 |
| 8 | 15.8 | 5.6 | 97 | 62 |
| 9 | 17.0 | 5.2 | 156 | 100 |
| 10 | 18.2 | 4.9 | 87 | 56 |
| 11 | 19.1 | 4.7 | 36 | 23 |
| 12 | 20.0 | 4.4 | 22 | 14 |
| 13 | 20.6 | 4.3 | 32 | 21 |
| 14 | 21.2 | 4.2 | 34 | 22 |
| 15 | 22.0 | 4.0 | 46 | 29 |
| 16 | 22.9 | 3.9 | 143 | 92 |
| 17 | 23.3 | 3.8 | 82 | 53 |
| 18 | 23.8 | 3.7 | 83 | 53 |
| 19 | 25.0 | 3.6 | 98 | 63 |
| 20 | 25.3 | 3.5 | 50 | 32 |
| 21 | 25.8 | 3.5 | 55 | 35 |
| 22 | 26.2 | 3.4 | 45 | 29 |
| 23 | 26.9 | 3.3 | 28 | 18 |
| 24 | 27.7 | 3.2 | 28 | 18 |
| 25 | 28.0 | 3.2 | 19 | 12 |
| 26 | 29.2 | 3.1 | 25 | 16 |
| 27 | 29.6 | 3.0 | 18 | 12 |
| 28 | 31.8 | 2.8 | 20 | 13 |
| 29 | 32.5 | 2.7 | 16 | 10 |
| 30 | 33.8 | 2.7 | 14 | 9 |
| 31 | 36.8 | 2.4 | 36 | 23 |
| 32 | 38.8 | 2.3 | 10 | 6 |

^{a}Bold: Unique set of XRPD Peaks for Form AC
^{b}Intensity of peak/Intensity of most intense peak × 100

TGA

TGA analysis indicated a two-step weight loss of 5.9% between 18° C. and 109° C. and 10.2% between 109° C. and 170° C.

¹H NMR

¹H NMR analysis indicated that the tiagabine hydrochloride Form AC contained 1.37 moles of cyclohexanol per mole of tiagabine HCl.

The citation and discussion of references in this specification is provided merely to clarify the description of the present invention and is not an admission that any such reference is “prior art” to the invention described herein. Each reference cited in this specification is incorporated herein by reference in its entirety.

What is claimed is:

1. A crystalline form of tiagabine chosen from tiagabine free base Form A, tiagabine free base Form B, tiagabine free base Form C, tiagabine free base Form D, tiagabine free base Form E, tiagabine free base Form F, tiagabine free base Form G, tiagabine free base Form H, tiagabine camphorate Form A, tiagabine hydrobromide Form A, tiagabine dl-malate Form A, tiagabine d-malate Form A, tiagabine tartrate Form A, tiagabine hydrochloride Form G, tiagabine hydrochloride Form K, tiagabine hydrochloride Form L, tiagabine hydrochloride Form N, tiagabine hydrochloride Form O, tiagabine hydrochloride Form R, tiagabine hydrochloride Form U, tiagabine hydrochloride Form V, tiagabine hydrochloride Form AC, and Crystalline Form A of tiagabine hydrochloride cocrystal with 2-furancarboxylic acid.

2. A crystalline form of tiagabine according to claim 1, wherein the crystalline form exhibits an x-ray powder diffraction pattern having characteristic peaks as set forth in the following table:

| Tiagabine | Form | Characteristic XRPD Peaks (±0.2 degrees 2θ) | | | | | | | | | |
|---|---|---|---|---|---|---|---|---|---|---|---|
| free base | A | 6.5 | 8.1 | 12.6 | 17.4 | 19.0 | 19.5 | 22.9 | 25.8 | 27.2 | — |
| free base | B | 15.0 | 15.4 | 17.3 | 21.3 | 22.5 | 24.8 | — | — | — | — |
| free base | C | 4.9 | 6.1 | 7.8 | 9.9 | 12.2 | 12.9 | — | — | — | — |
| free base | D | 5.7 | 6.1 | 10.0 | 12.2 | 15.8 | 16.9 | — | — | — | — |
| free base | E | 9.5 | 13.1 | 14.3 | 16.1 | 18.7 | 22.5 | — | — | — | — |
| free base | F | 6.3 | 8.0 | 10.0 | 10.5 | 16.2 | 21.1 | 21.8 | — | — | — |
| free base | G | 6.0 | 7.6 | 9.7 | 15.4 | 16.1 | 18.1 | 18.5 | 19.0 | 24.7 | — |
| free base | H | 15.8 | 16.8 | 20.7 | — | — | — | — | — | — | — |
| camphorate | A | 5.9 | 9.8 | 12.0 | 14.0 | 15.4 | 18.4 | 21.2 | — | — | — |
| hydrobromide | A | 3.9 | 7.8 | 12.8 | 14.2 | 14.4 | 15.7 | 21.5 | 21.8 | — | — |
| dl-malate | A | 4.2 | 11.3 | 11.9 | 15.5 | 15.9 | 18.7 | 19.2 | — | — | — |
| d-malate | A | 4.2 | 11.3 | 11.9 | 15.9 | 17.0 | 18.7 | 21.1 | 23.8 | — | — |
| Tartrate | A | 4.1 | 11.5 | 12.6 | 13.6 | 16.5 | 16.7 | 21.5 | 24.6 | — | — |
| hydrochloride | G | 3.9 | 14.7 | 16.0 | 16.9 | 20.5 | 25.5 | 28.1 | — | — | — |
| hydrochloride | K | 5.7 | 13.3 | 16.6 | 20.1 | 20.6 | 23.6 | 24.5 | 24.9 | — | — |
| hydrochloride | L | 7.7 | 12.5 | 14.5 | 17.1 | 21.1 | 21.8 | 24.6 | 25.1 | 26.2 | 28.0 |
| hydrochloride | N | 14.1 | 14.5 | 15.6 | 17.1 | 19.6 | 22.6 | 23.2 | 23.8 | 24.7 | 25.0 |
| hydrochloride | O | 12.6 | 14.6 | 16.4 | 18.6 | 18.9 | 23.3 | 24.3 | 25.9 | — | — |
| hydrochloride | R | 10.8 | 13.0 | 15.3 | 16.7 | 17.8 | 22.2 | 25.4 | 26.9 | 28.0 | 32.2 |
| hydrochloride | U | 12.6 | 14.4 | 16.4 | 16.9 | 21.2 | 21.6 | 22.9 | 23.9 | 26.6 | 27.6 |
| hydrochloride | V | 7.4 | 11.6 | 12.9 | 15.8 | 16.1 | 18.5 | 19.4 | 21.2 | 23.9 | 26.4 |
| hydrochloride | AC | 7.8 | 8.5 | 12.4 | 14.7 | 15.3 | 15.8 | 17.0 | 18.2 | 22.9 | 25.0 |
| hydrochloride co- | A | 7.5 | 11.6 | 14.7 | 17.2 | 21.7 | 22.9 | 26.6 | — | — | — |
| crystal with 2-furan- | | | | | | | | | | | |
| carboxylic acid | | | | | | | | | | | |

3. A crystalline form of tiagabine according to claim 1, wherein the crystalline form is chosen from tiagabine free base Forms A, B, C, D, E, F, G, and H, exhibiting an x-ray powder diffraction pattern having characteristic peaks as set forth in the following table:

| Form | Characteristic XRPD Peaks (±0.2 degrees 2θ) | | | | | | | | |
|---|---|---|---|---|---|---|---|---|---|
| A | 6.5 | 8.1 | 12.6 | 17.4 | 19.0 | 19.5 | 22.9 | 25.8 | 27.2 |
| B | 15.0 | 15.4 | 17.3 | 21.3 | 22.5 | 24.8 | — | — | — |
| C | 4.9 | 6.1 | 7.8 | 9.9 | 12.2 | 12.9 | — | — | — |
| D | 5.7 | 6.1 | 10.0 | 12.2 | 15.8 | 16.9 | — | — | — |
| E | 9.5 | 13.1 | 14.3 | 16.1 | 18.7 | 22.5 | — | — | — |
| F | 6.3 | 8.0 | 10.0 | 10.5 | 16.2 | 21.1 | 21.8 | — | — |
| G | 6.0 | 7.6 | 9.7 | 15.4 | 16.1 | 18.1 | 18.5 | 19.0 | 24.7 |
| H | 15.8 | 16.8 | 20.7 | — | — | — | — | — | — |

4. A crystalline form of tiagabine according to claim 1, wherein the crystalline form is a tiagabine salt chosen from tiagabine camphorate Form A, tiagabine hydrobromide Form A, tiagabine dl-malate Form A, tiagabine d-malate Form A, and tiagabine tartrate Form A, exhibiting an x-ray powder diffraction pattern having characteristic peaks as set forth in the following table:

| Tiagabine Salt | Form | Characteristic XRPD Peaks (±0.2 degrees 2θ) | | | | | | | |
|---|---|---|---|---|---|---|---|---|---|
| Tiagabine camphorate | A | 5.9 | 9.8 | 12.0 | 14.0 | 15.4 | 18.4 | 21.2 | — |
| Tiagabine hydrobromide | A | 3.9 | 7.8 | 12.8 | 14.2 | 14.4 | 15.7 | 21.5 | 21.8 |
| Tiagabine dl-malate | A | 4.2 | 11.3 | 11.9 | 15.5 | 15.9 | 18.7 | 19.2 | — |
| Tiagabine d-malate | A | 4.2 | 11.3 | 11.9 | 15.9 | 17.0 | 18.7 | 21.1 | 23.8 |
| Tiagabine tartrate | A | 4.1 | 11.5 | 12.6 | 13.6 | 16.5 | 16.7 | 21.5 | 24.6 |

5. A crystalline form of tiagabine according to claim 1, wherein the crystalline form is a tiagabine hydrochloride salt chosen from Forms G, K, L, N, O, R, U, V, and AC, exhibiting an x-ray powder diffraction pattern having characteristic peaks as set forth in the following table:

| Form | Characteristic XRPD Peaks (±0.2 degrees 2θ) | | | | | | | | | |
|---|---|---|---|---|---|---|---|---|---|---|
| G | 3.9 | 14.7 | 16.0 | 16.9 | 20.5 | 25.5 | 28.1 | — | — | — |
| K | 5.7 | 13.3 | 16.6 | 20.1 | 20.6 | 23.6 | 24.5 | 24.9 | — | — |
| L | 7.7 | 12.5 | 14.5 | 17.1 | 21.1 | 21.8 | 24.6 | 25.1 | 26.2 | 28.0 |
| N | 14.1 | 14.5 | 15.6 | 17.1 | 19.6 | 22.6 | 23.2 | 23.8 | 24.7 | 25.0 |
| O | 12.6 | 14.6 | 16.4 | 18.6 | 18.9 | 23.3 | 24.3 | 25.9 | — | — |
| R | 10.8 | 13.0 | 15.3 | 16.7 | 17.8 | 22.2 | 25.4 | 26.9 | 28.0 | 32.2 |
| U | 12.6 | 14.4 | 16.4 | 16.9 | 21.2 | 21.6 | 22.9 | 23.9 | 26.6 | 27.6 |
| V | 7.4 | 11.6 | 12.9 | 15.8 | 16.1 | 18.5 | 19.4 | 21.2 | 23.9 | 26.4 |
| AC | 7.8 | 8.5 | 12.4 | 14.7 | 15.3 | 15.8 | 17.0 | 18.2 | 22.9 | 25.0 |

6. A crystalline form of a tiagabine hydrochloride salt according to claim 5, wherein the crystalline form is chosen from Forms G, L, O and V.

7. A crystalline form of tiagabine according to claim 1, wherein the crystalline form has a purity of at least about 50% (w/w).

8. A crystalline form of tiagabine according to claim 2, wherein the crystalline form has a purity of at least about 50% (w/w).

9. A crystalline form of tiagabine according to claim 1, wherein the crystalline form is Crystalline Form A of tiagabine hydrochloride cocrystal with 2-furancarboxylic acid, exhibiting an x-ray powder diffraction pattern having characteristic peaks at 7.5, 11.6, 14.7, 17.2, 21.7, 22.9 and 26.6±0.2 degrees 2θ.

10. Tiagabine free base amorphous.

11. A pharmaceutical composition comprising one or more crystalline forms of tiagabine according to claim 1 and one or more pharmaceutically acceptable excipients.

12. A pharmaceutical composition comprising one or more crystalline forms of tiagabine free base according to claim 3 and one or more pharmaceutically acceptable excipients.

13. A pharmaceutical composition comprising one or more crystalline forms of a tiagabine salt according to claim 4 and one or more pharmaceutically acceptable excipients.

14. A pharmaceutical composition comprising one or more crystalline forms of a tiagabine hydrochloride salt according to claim 5 and one or more pharmaceutically acceptable excipients.

15. A pharmaceutical composition comprising one or more crystalline forms of a tiagabine hydrochloride salt according to claim 6 and one or more pharmaceutically acceptable excipients.

16. A pharmaceutical composition comprising Crystalline Form A of tiagabine hydrochloride cocrystal with 2-furancarboxylic acid according to claim 9 and one or more pharmaceutically acceptable excipients.

17. A pharmaceutical composition comprising tiagabine free base amorphous according to claim 10 and one or more pharmaceutically acceptable excipients.

18. A process for preparing a crystalline form of tiagabine comprising the steps of:
(a) crystallizing tiagabine free base from ethanol to provide tiagabine free base Form A; or
(b) slurrying tiagabine free base in a mixture of hexane, diisopropylether, and ethanol to provide tiagabine free base Form A; or
(c) drying tiagabine free base Form A under vacuum to provide tiagabine free base Form B; or
(d) crystallizing tiagabine free base from a solvent selected from isopropanol, acetonitrile, and ethanol to provide tiagabine free base Form C; or
(e) crystallizing tiagabine free base from a mixture of isopropanol and cyclohexane to provide tiagabine free base Form C; or
(f) crystallizing tiagabine free base from a mixture of methyl ethyl ketone and 2,2,2-trifluoroethanol to provide tiagabine free base Form C; or
(g) crystallizing tiagabine free base from a mixture of 2,2,2-trifluoroethanol and at least one solvent chosen from methyl ethyl ketone and isopropyl ether to provide tiagabine free base Form D; or
(h) crystallizing tiagabine free base from a mixture of propionitrile and t-butyl alcohol to provide tiagabine free base Form E; or
(i) crystallizing tiagabine free base from a mixture of methyl ethyl ketone and 2,2,2-trifluoroethanol to provide tiagabine free base Form E; or
(j) crystallizing tiagabine free base from acetonitrile to provide tiagabine free base Form E; or
(k) crystallizing tiagabine free base from a mixture of 2,2,2-trifluoroethanol, methyl ethyl ketone, and propyl ether to provide tiagabine free base Form E; or
(l) crystallizing tiagabine free base from a mixture of methanol and 2-propyl ether to provide tiagabine free base Form F; or
(m) crystallizing tiagabine free base from 2-butanol to provide tiagabine free base Form G; or
(n) crystallizing tiagabine free base from 1-propanol to provide tiagabine free base Form H; or
(o) preparing a solution of tiagabine free base and (+)-camphoric acid in methanol, and crystallizing tiagabine camphorate Form A from the solution; or
(p) preparing a solution of tiagabine free base and (+)-camphoric acid in methanol and acetonitrile or ethyl acetate, and crystallizing tiagabine camphorate Form A from the solution; or
(q) preparing a solution of tiagabine free base and hydrobromic acid in a mixture of ethyl acetate and acetonitrile, and crystallizing tiagabine hydrobromide Form A from the solution; or
(r) preparing a solution of tiagabine free base and hydrobromic acid in a mixture of ethyl acetate, acetonitrile and 2-propyl ether, and crystallizing tiagabine hydrobromide Form A from the solution; or
(s) preparing a solution of tiagabine free base and dl-malic acid in a mixture of ethyl acetate, acetonitrile and methanol, and crystallizing tiagabine dl-malate Form A from the solution; or
(t) preparing a solution of tiagabine free base and d-malic acid in a mixture of ethyl acetate and acetonitrile, and crystallizing tiagabine d-malate Form A from the solution; or
(u) preparing a solution of tiagabine free base and d-malic acid in a mixture of ethyl acetate, acetonitrile and methanol, and crystallizing tiagabine d-malate Form A from the solution; or
(v) preparing a solution of tiagabine free base and d-malic acid in a mixture of ethyl acetate and acetonitrile, crystallizing tiagabine d-malate Form A from the solution, and slurrying the crystallized tiagabine d-malate Form A in ether; or
(w) preparing a solution of tiagabine free base and L-(+)-tartaric acid in a mixture of methanol and acetonitrile, and crystallizing tiagabine tartrate Form A from the solution; or
(x) preparing a solution of tiagabine free base and L-(+)-tartaric acid in a mixture of methanol, acetonitrile and ethyl acetate, and crystallizing tiagabine tartrate Form A from the solution; or
(y) preparing a solution of tiagabine free base and L-(+)-tartaric acid in a mixture of acetone and ethyl acetate, and crystallizing tiagabine tartrate Form A from the solution; or
(z) preparing a solution of tiagabine free base and L-(+)-tartaric acid in a mixture of tetrahydrofuran and 2-propanol, and crystallizing tiagabine tartrate Form A from the solution; or
(aa) preparing a mixture of tiagabine hydrochloride and 2-furancarboxylic acid, and grinding the mixture to form crystalline Form A of tiagabine hydrochloride cocrystal with 2-furancarboxylic acid; or
(bb) preparing a mixture of tiagabine hydrochloride, 2-furancarboxylic acid and methanol, and grinding the mixture to form crystalline Form A of tiagabine hydrochloride cocrystal with 2-furancarboxylic acid; or
(cc) preparing a mixture of tiagabine hydrochloride monohydrate and 2-furancarboxylic, and grinding the mixture to form crystalline Form A of tiagabine hydrochloride cocrystal with 2-furancarboxylic acid; or
(dd) crystallizing tiagabine hydrochloride from chloroform to provide tiagabine hydrochloride Form G; or
(ee) crystallizing tiagabine hydrochloride from chloroform to provide tiagabine hydrochloride Form K; or
(ff) crystallizing tiagabine hydrochloride from nitromethane to provide tiagabine hydrochloride Form L; or
(gg) crystallizing tiagabine hydrochloride from benzonitrile to provide tiagabine hydrochloride Form N; or
(hh) heating tiagabine hydrochloride monohydrate to provide tiagabine hydrochloride Form O; or
(ii) slurrying tiagabine hydrochloride monohydrate in nitromethane to provide tiagabine hydrochloride Form R; or
(jj) slurrying tiagabine hydrochloride monohydrate in 1,2-dichloroethane to provide tiagabine hydrochloride Form U; or
(kk) slurrying tiagabine hydrochloride monohydrate in 1,2-dimethoxyethane to provide tiagabine hydrochloride Form V; or
(ll) crystallizing tiagabine hydrochloride from cyclohexanol to provide tiagabine hydrochloride Form AC.

19. A process for preparing an amorphous form of tiagabine free base comprising the step of:
(a) evaporating a solution of tiagabine free base in a solvent selected from 1,4-dioxane and isopropanol to provide tiagabine free base amorphous; or
(b) adding propyl ether to a solution of tiagabine free base in 1,4-dioxane to provide tiagabine free base amorphous; or
(c) precipitating tiagabine free base from an acetonitrile solution to provide tiagabine free base amorphous.
